(19) United States
(12) Patent Application Publication
(10) Pub. No.: US 2007/0059706 A1
(43) Pub. Date: Mar. 15, 2007

(54) Materials and methods relating to breast cancer classification

(76) Inventors: Kun Yu, Singapore (SG); Patrick Tan, Singapore (SG)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/574,392

(22) PCT Filed: Oct. 1, 2004
(86) PCT No.: PCT/GB04/04195

§ 371 Date: Nov. 22, 2006

(30) Foreign Application Priority Data
Oct. 3, 2003 (GB) 0323225.3

(51) Int. Cl. C12Q 1/68; G01N 33/574

(52) U.S. Cl. 435/6; 435/7.23

(57) ABSTRACT

The inventors set out to identify a set of genes for use as prognostic markers for breast tumours which correlate with the Nottingham Prognostic Index (NPI). Initially they were unable to identify a single set of genes whose expression correlates with the NPI. However after segregating the dataset into molecular subcategories (estrogen receptor positive, estrogen receptor negative, and ErbB2 positive) they identified a set of 62 genes which are differentially expressed in tumours of different prognoses. Methods and apparatus for determining prognosis are provided. Also provided are methods of determining the response of tumours to chemotherapy involving comparing the expression levels of the predictive gene set before and after treatment.

### FIELD OF THE INVENTION

The present invention concerns materials and methods relating to the classification of breast cancers. Particularly, the present invention concerns the determination of the prognosis of breast cancers.

### BACKGROUND OF THE INVENTION

There has been an intense interest in the use of gene expression data for biological classification, particularly in the fields of oncology and medicine. One exciting aspect of this approach has been its ability to define clinically relevant subtypes of cancer that have previously eluded more traditional light-microscopy approaches. Despite this potential, a number of issues have to be resolved before the use of gene expression data for clinical diagnosis can become a reality. For example, algorithms need to be implemented that, besides delivering the correct classification, can also accurately determine the confidence of the prediction. This is particularly important if the classification affects the subsequent course of treatment if furnished with such information, the treating physician can then weigh the confidence of prediction with the potential morbidity of a specific intervention to make an informed clinical choice.

The Nottingham Prognostic Index (NPI) is a classification system based on tumour size, histological grade, and lymph node status, which is widely used in Europe and the UK for assigning prognoses to breast tumours (1-5). Despite its utility, it is acknowledged that the use of conventional histopathological parameters such as tumour grade and cellular morphology are also associated with certain limitations. Many of these variables (e.g. grade) are subject to significant inter-observer variability even after standardization attempts (6). The NPI scale extends between values of 2 and 8. Appropriate cut-off points are often difficult to define when the parameter being measured is scored over a continuous range of values (7), such as the NPI.

The index therefore depends on a series of subjective criteria, which can result in discrepancies between observers in the assigned prognosis.

The NPI is a scale of values; a patient that has a lower NPI value than another patient typically has a better prognosis than that of the other patient. Prognosis is typically defined using factors such as the chance to survival over a particular timescale and/or chance of distant metastasis within a particular timescale (although not necessarily the same timescale as for survival). Generally speaking therefore, a patient's outlook decreases with increasing NPI value.

Determining a patient's prognosis is an important factor in determining the type and extent of treatment for the patient. As a future treatment program may be associated with prognosis, the accuracy of the assigned prognosis is therefore critical. For example, van't Veer et al. (10) have identified a 70 gene “prognosis expression signature” (PES) that predicts the Disease Free Survival (DFS) status of breast tumours.

### SUMMARY OF THE INVENTION

The present inventors studied expression data for a set of breast tumours but, initially, were unable to identify a set of genes whose expression is correlated to the NPI. The inventors hypothesized that there may be significant differences in gene expression between subtypes (“inter-subtype differences”), which potentially obscure more subtle patterns of variation within subtypes (“intra-subtype differences”). It has been proposed that a significant proportion of the intrinsic gene expression variation in breast cancer can be attributed to different tumours belonging to distinct ‘molecular subtypes’, such as ER+ and ER− (where ER is ‘Estrogen Receptor’)(8-9,14).

The dataset was segregated into respective molecular subcategories (ER+, ER−, ERBB2+) using unsupervised clustering techniques. Each molecular subtype was treated as an independent data set. Tumours within each subtype were independently analysed to define a set of genes whose level of expression correlates to the NPI.

Clinicians generally divide the NPI scale into three categories: ‘good’ prognosis, ‘moderate’ prognosis and ‘poor’ prognosis. The values that define the category boundaries vary depending on the clinician. An example of a typical set of boundaries is: good prognosis NPI<3.4; moderate prognosis 3.4=<NPI=<5.4; and poor prognosis NPI>5.4. Those skilled in the art will realise that these boundaries may be varied.

The present inventors have identified a set of 62 genes that are differentially expressed in tumours of differing prognoses, e.g. differentially expressed in tumours with a high NPI (and therefore poor prognosis) compared to tumours with a low NPI (and therefore good prognosis).

Although the set of genes was identified after classifying samples according to their NPI, it has also been found that classifying tumour samples using the expression levels of these genes correlates with other measures of prognosis (e.g. disease-free survival).

Accordingly, the expression levels of these genes in a tumour sample have significant medical implications for the prognosis and treatment of the patient from whom the sample was derived. In particular, they may be used to classify a tumour sample, as an indicator of the prognosis of the patient.

Values ranging from 3.8 to 4.6 on the NPI scale were used as cut-off points between “good” and “bad” prognosis and the same set of 62 differentially expressed genes were identified using each cut-off value.

This indicates that, although NPI covers a continuous spectrum of values from 2 to 8, the expression levels of genes from the set of 62 genes are capable of classifying tumour samples into discrete categories. Thus, samples exhibiting continuous NPI values based upon histopathological parameters may be separable into discrete categories at the molecular level.

Moreover, comparison of prognoses assigned to breast tumour patients using (i) the methods of the invention and (ii) clinical techniques (usually histopathological techniques), indicates that, based on patient data such as DFS and Kaplan-Meier survival curves, the methods of the invention may provide a more accurate prognosis than histopathological techniques.

The 62 genes are identified in Table S6. The following description will make use of the term “expression profile”. This refers to the expression levels for a set of genes in a sample. Unless the context requires otherwise, the set of genes will include some or all of the 62 genes identified in Table S6.

The 62 genes identified herein overlap by one gene only (DC13 or Hs. 6879) with the genes identified in the PES of van't Veer et al. (10). The PES is the first 70 genes (the genes that exhibit the most significant difference in expression between groups showing different disease free survival rates) of an extended gene set of 231 Rosetta genes (10). There are 8 genes common to the 62 genes of Table S6 and the 231 Rosetta genes, which eight genes are listed in Table S13.

Two genes in table S6 are highly expressed in low NPI tumours (the “Negative genes”), whilst 60 of the genes are highly expressed in high NPI tumours (the “Positive genes”).

Accordingly, at its most general, the present invention provides a method for deriving a set of differentially expressed genes. The invention also provides methods and assays for the classification and/or assignment of a prognosis to a breast tumour sample. The invention identifies a set of genes and provides the use of the expression levels of some or all of those genes in a breast tumour sample in assigning a prognosis to the patient from whom the sample was derived.

In a first aspect, the present invention provides a method for determining the prognosis of a patient with breast cancer, the method comprising assigning a prognosis to the patient based on the expression levels in a breast tumour of said patient of a set of genes (hereafter referred to as the “prognostic set”), wherein the prognostic set includes a plurality of genes from Table S6.

The invention further provides the use of the prognostic set in determining the prognosis of a patient with breast cancer. Preferably, the invention provides the use of an expression profile in determining the prognosis of a patient with a breast tumour, the expression profile representing the expression levels in the tumour of the genes of the prognostic set.

“Prognosis” is intended in its most general sense, and may be quantitative or qualitative. It may be expressed in general terms, such as a “good” or “bad” prognosis, and/or in terms of likely clinical outcomes, such as duration of disease free survival (DFS), likelihood of survival for a defined period of time, and/or probability of distant metastasis within a defined period of time. Quantitative measures of prognosis will generally be probabilistic. Additionally or alternatively, and especially for communicating the prognosis to or between medical practitioners, the prognosis may be expressed in terms of another indicator of prognosis, such as the NPI scale.

In general, a patient with a ‘good prognosis’ tumour would probably be treated with a conventional treatment regimen. A patient with a ‘poor prognosis’ tumour might be treated with an alternative or more aggressive regimen. The ‘poor prognosis’ patient would usually not have to wait for the conventional treatment regimen to fail before moving onto the more aggressive one. Furthermore, having an understanding of the likely clinical course of the disease allows a patient to prepare a realistic plan for future, which is an important social aspect of cancer treatment.

For the avoidance of doubt, the term “determining” need not imply absolute certainty in prognosis. Rather, the expression levels of the prognostic set in a tumour will generally be indicative of the likely prognosis of the patient.

The expression levels will generally be represented numerically. The expression profile therefore will generally include a set of numbers, each number representing the expression level of a gene of the prognostic set.

A method in accordance with the first aspect of the invention may comprise the steps of:
providing an expression profile that represents the expression levels in the tumour of the genes of the prognostic set, and
assigning a prognosis to the patient based on the expression profile.

The providing step may include extracting information on the expression levels of the genes of the prognostic set from a pre-existing data set, which may also include other expression levels (e.g. data representing expression levels of other genes in the tumour). Alternatively, it may include determining the expression levels experimentally.

The determining step may include the steps of:
(a) obtaining a breast tumour sample from the patient;
(b) measuring the expression levels in the sample of the genes of the prognostic set.

Measurement of the expression level of a gene, and in particular its representation in the expression profile, may be in absolute terms, or relative to some other factor such as, but not limited to, the expression to another gene, or a mean, median or mode of the expression level of a group of genes (preferably genes outside the prognostic set, but possibly including genes of the prognostic set) in the sample or across a group of samples. For example, expression of a gene may be measured or represented as a multiple or fraction of the average expression of a plurality of genes in the sample. Preferably, the expression is represented in the expression profile as positive or negative to indicate an increase or decrease in expression relative to the average value.

In a non-preferred embodiment, expression profile information in the form of a set of numerical values is converted into a ranked list of genes of the prognostic set, wherein the genes are ranked in order of expression level, after which the rank order of the individual genes is used as a parameter in the analysis (instead of the expression value of the gene).

Preferably, step (b) comprises contacting said expression products obtained from the sample with a plurality of binding members capable of binding to expression products that are indicative of the expression of genes of the prognostic set, wherein such binding may be measured.

Generally, the binding members are capable of not only detecting the presence of an expression product but its relative abundance (i.e. the amount of product available). The expression profile can be determined using binding members capable of binding to the expression products of the prognostic set, e.g. mRNA, corresponding cDNA or cRNA or expressed polypeptide. By labelling either the expression product or the binding member it is possible to identify the relative quantities or proportions of the expression products and determine the expression profile of the prognostic set. The binding members may be complementary nucleic acid sequences or specific antibodies.

The step of assigning a prognosis may be carried out by comparing the expression profile under test with other, previously obtained, profiles that are associated with known prognoses and/or with a previously determined “standard” profile (or profiles) which is (or are) characteristic of a particular prognosis (or prognoses). A standard profile for a particular prognosis may be generated from expression profiles from a plurality of tumours of that prognosis.

The comparison will generally be performed by, or with the aid of, a computer.

Preferably the expression profile is compared with known or standard profiles (preferably standard profiles) of differing known prognoses. The prognosis to be assigned to the patient is that of the known or standard profile which the expression profile under test most closely resembles.

Preferably the comparison is with known or standard profiles (preferably standard profiles) that are categorised into two different prognoses, e.g. “good” and “bad”, or high and low NPI (preferably with a cut-off between 3.8 and 4.6). The known or standard profiles will have been generated from samples of known prognosis, which may be determined in any convenient way—either by actual clinical outcome for the patient following the removal of the sample, or by other prognostic techniques, e.g. histopathological techniques, e.g. using the NPI scale.

The comparison may involve an assessment of the confidence level attributable to the prognosis, based on statistical techniques. The standard profiles are usually specific to the particular materials and methods (e.g. microarray) from which they were derived. If a new materials and/or methods (e.g. a new type of microarray) are adopted, the standard profiles of known prognoses are preferable obtained again using the prognostic set.

The method according to the first aspect of the invention may include classifying the sample of breast tumour as being of either high NPI or low NPI, or as either, of good or bad prognosis, for example.

As mentioned previously, the step of assigning a prognosis may be carried out by comparing the expression profile from the breast tumour sample under test with previously obtained profiles and/or a previously determined “standard” profile which is characteristic of a particular prognosis, for example, a ‘good’ and/or a ‘poor’ prognosis and/or at least one NPI value and/or at least one range of NPI values. The previously obtained profiles may be stored as a database of profiles.

Preferably the database includes gene expression profiles characteristic of a particular prognosis. The gene expression profiles are preferably produced from expression levels of the same prognostic set (a subset of the genes of Table S6) as the prognostic set of the first aspect of the invention, or a prognostic set (potentially a different subset from above) sufficiently overlapping the prognostic set of the first aspect so as to provide a statistically significant base for comparison of the expression levels. The computer may be programmed to report the statistical similarity between the profile under test and the standard profile(s) so that a prognosis may be assigned.

Advantageously, the use of a gene expression profile to assign a prognosis may reduce or may even eliminate the subjective nature of the clinical procedures used to assign a prognosis to a tumour sample. As the method requires assessment of expression products at the molecular level, preferably quantitatively, the method provides a more objective, and therefore potentially more reliable, way to assign a prognosis. The prognostic set is, as mentioned earlier, capable of separating breast tumour samples into discrete categories, and therefore reducing, or even eliminating, the subjective analysis of clinical prognostic assignment. Furthermore, a confidence can be assigned to the prediction, so that an informed choice regarding treatment of the patient can be made, depending on the “strength” of the prognosis.

The expression profile of the prognostic set may differ slightly between independent samples of similar prognosis. However, the inventors have realised that the expression profile of the particular genes that make up the prognostic set when used in combination provide a pattern of expression (expression profile) in a tumour sample, which pattern is characteristic of the tumour's prognosis.

The inventors have found that the prognostic set is capable of resolving tumour samples into high NPI and low NPI classes. By high NPI it is meant an NPI of preferably at least 3.4, preferably at least 3.5, more preferably at least 3.6, more preferably at least 3.7, more preferably at least 3.8, more preferably at least 3.9 and most preferably at least 4.0. High NPI may be at least 4.1, at least 4.2, at least 4.3, at least 4.4, at least 4.5, or at least 4.6. The preferred cut-off value between high and low NPI is between 3.8-4.6.

Historically, the ‘good’, ‘moderate’ and ‘bad’/‘poor’ categories of NPI were determined using large clinical studies in which patients belonging to these different groups exhibited statistically significant differences in overall survival. For example, patients with good prognosis may have a ten-year survival rate of about 83%, patients with ‘moderate’ prognosis may have a ten-year survival rate of about 52%, and patients with ‘poor’ or ‘bad’ prognosis may have a ten-year survival rate of about 13% (4).

In particular, the prognostic set seems to be correlated most strongly to tumour prognosis (as reflected by NPI) in Estrogen Receptor positive tumours (ER+).

The classification of breast tumours into Estrogen Receptor positive (ER+) and negative (ER−) subtypes is an important distinction in the treatment of breast cancer. ER− tumours are in general more clinically aggressive than their ER+ counterparts, and ER+ tumours are routinely treated using anti-hormonal therapies such as tamoxifen (21). Breast tumours may be classified as ER+ or ER− using histological techniques (e.g. with antibodies specific for the receptor) or using gene expression techniques. Presently, a tumour's ER status is routinely determined by immunohistochemistry (IHC) or immunoblotting using an antibody to ER.

The first aspect of the invention preferably includes a step of determining the ER status of the tumour sample. The ER status may be determined using gene expression analysis, or by using histopathological techniques. Preferably, the first, aspect of the invention further includes, as an initial step, determining the ER status of the tumour sample, and proceeding only if the status is ER+.

Preferably the ER status of the tumour sample is determined using gene expression profiling as described in our co-pending application PCT/GB03/000755. Gene expression profiling is capable of classifying breast tumours as ER+ or ER−, with high confidence. However, there is also a third category of tumours that could not be classified as ER+ or ER− with significant statistical certainty (‘low confidence’ tumours). Upregulation of ERBB2+ is frequently associated with low confidence tumours. Preferably, only ER+ tumours identified with high confidence (preferably classified as ER+ with a prediction strength of magnitude greater than 0.4 as determined using the methods of PCT/GB03/000755) are assessed using the methods according to the first aspect of the invention.

The step of assigning a prognosis to the breast tumour sample may comprise the use of statistical and/or probabilistic techniques, such as Weighted Voting (WV) (13), a supervised learning technique. In WV, binary classifications may be performed. That is, the technique may be used to assign a sample to one of two classes. The expression level of each gene in the prognostic set of the breast tumour sample is compared to the mean average level of expression of that gene across the different classes. The mean average may, for example, be calculated from expression profiles that have an assigned prognosis, e.g. database of expression profiles of ‘known’ prognosis.

The difference between the expression level and the mean average gene expression across the classes is weighted and corresponds to a ‘vote’ for that gene for a particular class and an equal, but negative, vote for that gene against the other class. For a particular tumour, the votes (positive and negative) for all the genes are summed together for each class to create totals for each class. The tumour is assigned to the class having the highest (positive) total. The margin of victory of the winning class can then be expressed as prediction strength.

The difference in expression level is weighted using a formula that includes mean and standard deviations of expression levels of the genes in each of the two classes. Generally, the mean and standard deviations for each class are calculated from expression profiles that have, or represent, a particular prognosis e.g. high NPI and low NPI.

Additionally, or alternatively, the step of assigning a prognosis may comprise the use of hierarchical clustering, particularly if expression levels in the tumour sample have been determined using different materials and/or methods from those used to determine the expression profiles with ‘known’ prognoses, or standard profile(s) to which the sample expression profile is compared.

The assigned prognosis may be validated using an established leave-one-out cross validation (LOOCV) assay (see examples). Step (c) may be performed using a computer.

In Hierarchical Clustering, each expression profile can be represented as a vector that consists of n genes where (g1, g2 . . . gn) represent the expression levels of the genes. Each vector is then compared with the vector for every other profile in the analysis, and the two vectors with the highest correlation to one another are paired together until as many profiles as possible in the analysis have been paired up.

There are many ways known in the art to calculate the correlation, such as the Pearson's correlation coefficient (22). In the next step, a composite vector is then derived from each pair (in average-linkage clustering this is usually the average of both profiles), and then the process of pairing is repeated. This continues until all vectors have been paired together, to assemble a “tree” representing all the profiles. The process is ‘hierarchical’ as one starts from the bottom (individual profiles) and builds up. In the present invention, individual profiles build up to preferably two composite vectors, each vector representing a class (i.e. good or bad prognosis). For a new sample of unknown class, the sample is clustered with the standard profiles/samples. The class of ‘unknown’ sample will be determined based on which cluster/vector it belongs to at the end of the iterative rounds of pairing.

By expression profiles with ‘known’ or assigned prognosis/prognoses, it is meant an expression profile to which a prognosis has been assigned or derived. The prognosis may have been: calculated from gene expression data; derived from clinical techniques performed on the source sample (e.g. histopathological techniques); or assigned retrospectively based on the actual disease progression/outcome in the patient from which the expression profile was derived. The third option is most preferable, as an accurate prognosis (for the point in time at which the sample was obtained) can be assigned, based on the subsequent outcome for the patient, from the patient's medical records. In such retrospective assignment, the use of hindsight provides accuracy.

The methods of the invention may be used to assess the efficacy of treatment of a patient with breast cancer. The prognosis of the patient may be assigned before, or at an early stage of, treatment and compared to the prognosis assigned to the patient after treatment (or at a late stage of treatment). The prognosis before and/or after treatment is preferably assigned using a method according to the invention. If the treatment comprises stages, the expression profile may be determined after each stage to plot the progress of the treatment. An improved prognosis after treatment indicates a successful, or at least partially successful, treatment. The treatment may be chemotherapy.

The methods of the invention may include comparing the expression levels of the prognostic set in the breast tumour sample before and after treatment to detect a change in the expression profile indicative of an improved prognosis or worsened prognosis.

The method may include detecting downregulation of genes in the prognostic set that are indicated in Table S6 to be ‘upregulated’ and/or upregulation of genes in the prognostic set that are indicated in Table S6 to be ‘downregulated’. The said genes may be downregulated/upregulated compared to standard values (e.g. the average expression level across a range of samples of differing prognosis), and/or compared to previous values, for example a standard profile indicative or characteristic of a ‘poor’ prognosis. The downregulation of the ‘upregulated’ genes and/or upregulation of the ‘downregulated’ genes is indicative of a good or moderate prognosis. The extent of the change in regulation may indicate the efficacy of the treatment.

The inventors have found that a change in expression profile towards that of a good prognosis tumour is indicative of successful treatment. Tumours that exhibit such a change in expression profile have the best prognosis (e.g. the best survival rates, the best disease free survival rates). The expression profile of the tumour at pre- and post-treatment stages may be compared to standard profiles of known prognosis.

The method may therefore comprise assigning the expression profile of a breast tumour to either good or bad prognosis class (or high or low NPI class), and assigning a second expression profile, determined from said tumour at a later stage of treatment, to either good or bad prognosis class (or high or low NPI class), and detecting a change in class, wherein a change from bad prognosis to good prognosis (or high NPI to low NPI) is indicative of an effective treatment. Additionally, or alternatively, a change in the statistical confidence level of assignment of good or bad prognosis class (or high or low NPI class) may indicate the efficacy of treatment. A decrease in the confidence of assignment of a class indicative of poor prognosis may suggest a successful, or at least partially successful, treatment.

The methods of assessing the efficacy of treatment may include the step of determining the ER status of the tumour.

However, the said methods of assessing efficacy are effective for assessing treatment efficacy of ER+, ER− and ERBB2+tumours i.e. irrespective of the ER status of the tumour.

The expression profile represents the expression levels of a group of genes in the tumour. The genes of each expression profile need not be identical but there should be sufficient overlap between the genes of each expression profile to allow comparison and grouping of the expression profiles.

The binding member may be labelled for detection purposes using standard procedures known in the art. Alternatively, the expression products may be labelled following isolation from the sample under test. A preferred means of detection is using a fluorescent label which can be detected by a light meter. Alternative means of detection include electrical signalling. For example, the Motorola (Pasadena, Calif.) e-sensor system has two probes, a “capture probe” which is freely floating, and a “signalling probe” which is attached to a solid surface which doubles as an electrode surface. Both probes function as binding members to the expression product. When binding occurs, both probes are brought into close proximity with each other resulting in the creation of an electrical signal which can be detected.

There are, however, a number of newer technologies that have recently emerged that utilize ‘label-free’, techniques for quantitation, for example those produced by Xagros (Mountain View, Calif.). The primers and/or the amplified nucleic acid may be devoid of any label. Quantitation may be assessed by measuring the change in electrical resistance as a result of two primers docking onto a target expressed product, and subsequent extension by polymerase.

As discussed above, the binding members may be oligonucleotide primers for use in a PCR (e.g. multi-plexed PCR) to amplify specifically the number of expressed products of the genetic identifiers. The products would then be analysed on a gel. However, preferably, the binding member is a single nucleic acid probe or antibody fixed to a solid support. The expression products may then be passed over the solid support, thereby bringing them into contact with the binding member. The solid support may be a glass surface, e.g. a microscope slide; beads (Lynx); or fibre-optics. In the case of beads, each binding member may be fixed to an individual bead and they are then contacted with the expression products in solution.

Various methods exist in the art for determining expression profiles for particular gene sets and these can be applied to the present invention. For example, bead-based approaches (Lynx) or molecular bar-codes (Surromed) are known techniques. In these cases, each binding member is attached to a bead or “bar-code” that is individually readable and free-floating to ease contact with the expression products. The binding of the binding members to the expression products (targets) is achieved in solution, after which the tagged beads or bar-codes are passed through a device (e.g. a flow-cytometer) and read.

A further known method of determining expression profiles is instrumentation developed by Illumina (San Diego, Calif.), namely, fibre-optics. In this case, each binding member is attached to a specific “address” at the end of a fibre-optic cable. Binding of the expression product to the binding member may induce a fluorescent change which is readable by a device at the other end of the fibre-optic cable.

The present inventors have successfully used a nucleic acid microarray comprising a plurality of nucleic acid sequences fixed to a solid support. By passing nucleic acid sequences representing expressed genes e.g. cDNA, over the microarray, they were able to create a binding profile characteristic of the expression products from a tumour sample with a particular prognosis, in particular a tumour sample with a good prognosis or a tumour sample with a bad prognosis or a tumour sample with a high NPI or a tumour sample with a low NPI.

In a second aspect, the present invention provides apparatus, preferably a microarray, for assigning a prognosis to a breast tumour sample, which apparatus comprises a solid support to which are attached a plurality of binding members, each binding member being capable of specifically binding to an expression product of a gene of the prognostic set. Preferably the binding members attached to the solid support are capable of specifically and independently binding to expression products of at least 5 genes, more preferably, at least 10 genes or at least 15 genes, and most preferably at least 20 or 30 genes identified in Table S6. The binding members attached to the solid support may be capable of specifically binding to expression products of 20 to 30 genes identified in Table S6.

In one embodiment, binding members being capable of specifically and independently binding to expression products of all genes identified in Table S6 are attached to the solid support. The support may have attached thereto only binding members that are capable of specifically and independently binding to expression products of the genes identified in Table S6, or a prognostic set therefrom.

The apparatus preferably includes binding members capable of specifically binding to expression products from the prognostic set, or to a plurality of genes thereof, and may include binding members capable of specifically binding to expression products of only an incomplete subset of the genes that are represented on the U133A microarray (though it may also include binding members for other genes not represented on the U133A microarray). It is believed that the U133A microarray represents about 14397 distinct genes. Accordingly, the apparatus preferably includes binding members for no more than 14396 of the genes on the U133A microarray. The apparatus may include binding members capable of specifically binding to expression products of no more than 90% of the genes on the U133A microarray. The apparatus may include binding members-capable of specifically binding to expression products of no more than 80% or 70% or 50% or 40% or 30% or 20% or 10% or 5% of the genes on the U133A microarray.

Additionally or alternatively, the solid support may house binding members for no more than 14000, or no more than 10000, or no more than 5000, or no more than 3000, or no more than 1000, or no more than 500, or no more than 400, or no more than 300, or no more than 200, or no more than 100, or no more than 90, or no more than 80, or no more than 70, or no more than 60, or no more than 50, or no more than 40, or no more than 30, or no more than 20, or no more than 10, or no more than 5 different genes.

Preferably the binding members are nucleic acid sequences and the apparatus is a nucleic acid microarray.

The genes of Table S6 are listed with their Unigene accession numbers corresponding to Build 160 of the Unigene database. The sequence of each gene can therefore be retrieved from the Unigene database at the National Institute of Health (NIH): (http://www.ncbi.nlm.nih.gov/entrez/guery.fcqi?db=unigene).

Furthermore, for all of the genes, Affymetrix (Santa Clara, Calif.)(www.affymetrix.com) provide examples of probe sets, including the sequences of the probes, (i.e. binding members in the form of oligonucleotide sequences) that are capable of detecting expression of the gene when used on a solid support. The probe details are accessible from the U133A section of the Affymetrix website using the Unigene ID of the target gene.

If, in the future, one of the Unigene ID's listed in the table were to be merged into a new ID, or split into two or more ID's (e.g. in a new build of the database) or deleted altogether, the sequence of the gene, as intended by the present inventors, is retrievable by accessing Build 160 of Unigene.

Typically, high density nucleic acid sequences, usually cDNA or oligonucleotides, are fixed onto very small, discrete areas or spots of a solid support. The solid support is often a microscopic glass side or a membrane filter, coated with a substrate (i.e. a “chip”). The nucleic acid sequences are delivered (or printed), usually by a robotic system, onto the coated solid support and then immobilized or fixed to the support.

In a preferred embodiment, the expression products derived from the sample are labelled, typically using a fluorescent label, and then contacted with the immobilized nucleic acid sequences. Following hybridization, the fluorescent markers are detected using a detector, such as a high resolution laser scanner. In an alternative method, the expression products could be tagged with a non-fluorescent label, e.g. biotin. After hybridisation, the microarray could then be ‘stained’ with a fluorescent dye that binds/bonds to the first non-fluorescent label (e.g. fluorescently labelled strepavidin, which binds to biotin). The expression products may, however, be label-free, as discussed above.

A binding profile indicating a pattern of gene expression (expression pattern or profile) is obtained by analysing the signal emitted from each discrete spot with digital imaging software. The pattern of gene expression of the experimental sample may then be compared with that of a standard profile (i.e. an expression profile from a tissue sample with, for example, a known good or bad prognosis, or a known NPI value or known range of NPI values) for differential analysis.

The standard may be derived from one or more expression profiles previously judged to be characteristic of a particular prognosis e.g. ‘poor’ or ‘good’ prognosis and/or of a particular NPI range such as high and/or low NPI and/or characteristic of one or more NPI value(s) or one or more range(s) of values. The standard may be derived from one or more expression profiles previously judged to be characteristic of a particular NPI value or range of values (or other defined value on a prognostic scale). The standard may include an expression profile characteristic of a normal sample. These/This standard expression profile(s) may be retrievably stored on a data carrier as part of a database.

Most microarrays utilize either one or two fluorophores. For two-colour arrays, the most commonly used fluorophores are Cy3 (green channel excitation) and Cy5 (red channel excitation). The object of the microarray image analysis is to extract hybridization signals from each expression product. For one-colour arrays, signals are measured as absolute intensities for a given target (essentially for arrays hybridized to a single sample). For two-colour arrays, signals are measured as ratios of two expression products, (e.g. sample and control (controls are otherwise known as a ‘reference’)) with different fluorescent labels.

The apparatus in accordance with the present invention preferably comprises a plurality of discrete spots, each spot containing one or more oligonucleotides and each spot representing a different binding member for an expression product of a gene selected from Table S6. In one embodiment, the microarray will contain spots for each of the genes provided in Table S6. Each spot will comprise a plurality of identical oligonucleotides each capable of binding to an expression product, e.g. mRNA or cDNA, of the gene of Table S6 it is representing. Each gene is preferably represented by a plurality of different oligonucleotides, preferably the Affymetrix U133A set of probes for the gene.

In a third aspect of the present invention, there is provided a kit for assigning a prognosis to a patient with breast cancer, said kit comprising a plurality of binding members capable of specifically binding to expression products of genes of the prognostic set, and a detection reagent. The kit may include a data analysis tool, preferably in the form of a computer program. The data analysis tool preferably comprises an algorithm adapted to discriminate between the expression profiles of tumours with differing prognoses. Preferably the algorithm is adapted to discriminate between a ‘good’ prognosis and a ‘poor’ prognosis, most preferably between high NPI and low NPI tumours. The algorithm is preferably a weighted voting algorithm as described above.

In one embodiment, the kit includes apparatus of the second aspect of the invention.

The kit may include expression profiles from breast tumour samples with known prognoses (as discussed above), and/or gene expression profiles characteristic of a particular prognosis (as discussed above), preferably stored on a data carrier or other memory device. The profiles may have been analysed or grouped statistically, for example, mean average expression levels and/or gene weightings calculated.

Preferably, the one or more binding members (antibody binding domains or nucleic acid sequences e.g. oligonucleotides) in the kit are fixed to one or more solid supports e.g. a single support for microarray or fibre-optic assays, or multiple supports such as beads. The detection means is preferably a label (radioactive or dye, e.g. fluorescent) for labelling the expression products of the sample under test. The kit may also comprise reagents for detecting and analysing the binding profile of the expression products under test.

Alternatively, the binding members may be nucleotide primers capable of binding to the expression products of genes identified in Table S6 such that they can be amplified in a PCR. The primers may further comprise detection means, i.e. labels that can be used to identify the amplified sequences and their abundance relative to other amplified sequences.

The breast tumour sample may be obtained as excisional breast biopsies or fine-needle aspirates.

By creating a number of expression profiles of the prognostic set from a number of tumour samples, each with an assigned prognosis, preferably based on a prognostic scale, it is possible to create a library of profiles for good and bad prognosis. The greater the number of expression profiles, the easier it is to create a reliable characteristic expression profile standard (i.e. including statistical variation) that can be used as a standard in a prognostic assay. Thus, a standard profile may be one that is devised from a plurality of individual expression profiles and devised within statistical variation to represent, for example, a ‘good’ or ‘poor’ prognosis, or a high NPI or a low NPI.

In a fourth aspect, there is provided a method of producing a nucleic acid expression profile for a breast tumour sample comprising the steps of
(a) isolating expression products from said breast tumour sample;
(b) identifying the expression levels of the prognostic set of genes; and
(c) producing from the expression levels an expression profile for said breast tumour sample.

The expression profile may be added to a gene expression profile database. The method may further comprise the step of comparing the expression profile with a second expression profile (or a plurality of second expression profiles). The second expression profile (or profiles) may be produced from a second breast tumour sample (or samples) using substantially the same prognostic set, wherein a prognosis has been assigned to, or determined for, the second sample (or samples). The second expression profile (or profiles) may be a standard profile (or profiles) characteristic of a particular prognosis, for example a ‘good’ prognosis or a ‘poor’ prognosis, or a high NPI or a low NPI, or at least one particular NPI value or at least one range of NPI values.

Preferably the prognosis is in the form of a prognostic measure, preferably a clinically accepted prognostic classification system, such as the NPI. Again, the prognosis may be predicted from gene expression data, derived from clinical techniques, such as histopathological techniques, or assigned retrospectively to the second expression profile based on the disease outcome of the patient(s) that contributed sample(s) from which the second profile was derived.

With knowledge of the prognostic set, it is possible to devise many methods for determining the expression pattern or profile of the genes in a particular test sample. For example, the expressed nucleic acid (RNA, mRNA) can be isolated from the sample using standard molecular biological techniques. The expressed nucleic acid sequences corresponding to the gene members of the genetic identifiers given in Table S6 can then be amplified using nucleic acid primers specific for the expressed sequences in a PCR. If the isolated expressed nucleic acid is mRNA, this can be converted into cDNA for the PCR reaction using standard methods.

The primers may conveniently introduce a label into the amplified nucleic acid so that it may be identified. Ideally, the label is able to indicate the relative quantity or proportion of nucleic acid sequences present after the amplification event, reflecting the relative quantity or proportion present in the original test sample. For example, if the label is fluorescent or radioactive, the intensity of the signal will indicate the relative quantity/proportion or even the absolute quantity, of the expressed sequences. The relative quantities or proportions of the expression products of each of the genetic identifiers will establish a particular expression profile for the test sample.

The method according to the fourth aspect of the invention may comprise the steps of:
(a) isolating expression products from a first breast tumour sample; contacting said expression products with a plurality of binding members capable of specifically and independently binding to expression products of the prognostic set; and creating a first expression profile from the expression levels of the prognostic set in the tumour sample;
(b) isolating expression products from a second breast tumour sample of known prognosis (as defined previously); contacting said expression products with a plurality of binding members capable of specifically and independently binding to expression products of the prognostic set of step (a), so as to create a comparable second expression profile of a breast tumour sample; (c) comparing the first and second expression profiles to determine the prognosis of the first breast tumour sample.

In a fifth aspect of the invention, there is provided an expression profile database comprising a plurality of gene expression profiles of breast tumour samples, wherein the gene expression profiles are derived from the expression levels of the prognostic set of genes, which database is retrievably held on a data carrier. The database is preferably produced by the method according to the fourth aspect of the invention.

The expression profiles are preferably nucleic acid expression profiles. The determination of the nucleic acid expression profile may be computerised and may be carried out within certain previously set parameters, to avoid false positives and false negatives.

The database may include expression profiles characteristic of a particular prognosis, such as good or bad prognosis, or of a particular prognostic value, preferably NPI value (e.g. high NPI, low NPI, or specific qualitative value or range of values). The expression profiles may be categorised, according to the ER status (i.e. ER+ or ER−) of the source tumour. The database may then be processed and analysed such that it will eventually contain (i) the numerical data corresponding to each expression profile in the database, (ii) a “standard” profile which functions as the canonical profile for a particular prognostic assignment (e.g. good or bad prognosis, or value or range of values, preferably from the NPI); and (iii) data representing the observed statistical variation of the individual profiles to the “standard” profile.

The computer may then be able to provide an expression profile standard characteristic of a breast tumour sample with a particular prognosis, e.g. good prognosis and/or bad prognosis and/or a high NPI and/or a low NPI. As stated earlier, the determined expression profiles may then be used to assign a prognosis to the breast tissue sample, preferably using a discriminating algorithm, most preferably a Weighted Voting algorithm, described above.

The classification of the expression profile is more reliable the greater number of gene expression levels tested. The known microarray and genechip technologies allow large numbers of binding members to be utilized. Therefore, the more preferred method would be to use binding members representing all of the genes in Table S6. However, the skilled person will appreciate that a proportion of these genes may be omitted and the method still carried out in a reliable and statistically accurate fashion.

The prognostic set in any aspect of the invention may comprise, or consist of, all, or substantially all, of the genes from Table S6, or all, or substantially all of the Positive genes and/or all of the Negative genes. The prognostic set of genes may vary in content and number, independently, between aspects of the invention.

The prognostic set may include at least 5, 10, 20, 30, 40, 50, 60 or all of the genes of Table S6.

Preferably, the said prognostic set comprises, or consists of, about sixty or about fifty or about forty or about thirty or about twenty or about ten or about five Positive genes from Table S6. Positive genes from Table S6 are preferably selected from the upper portion, preferably the upper half, of the list of Positive genes in Table S6, as the genes are ranked in order of significance.

The prognostic set may comprise one or both of, or may consist of both of, the Negative genes from Table S6.

The number and choice of genes are selected so as to provide a prognostic set that is at least capable of distinguishing between tumours with good prognosis and tumours with bad prognosis (or tumours with high NPI and tumours with low NPI).

The prognostic set may include no more than sixty genes of Table S6. The prognostic set may comprise no more than fifty genes of Table S6. The prognostic set may include no more than forty genes of Table S6. The prognostic set may include no more than thirty genes of Table S6. The prognostic set may include no more than twenty genes of Table S6. The prognostic set may include no more than ten genes of Table S6. The prognostic set may include no more than five genes of Table S6.

The prognostic set may comprise, or consist essentially of, five to sixty genes of Table S6. The prognostic set may comprise, or consist essentially of, ten to forty genes of Table S6. The prognostic set may comprise, or consist essentially of, ten to thirty genes of Table S6. The prognostic set may comprise, or consist essentially of, ten to twenty genes of Table S6, or twenty to thirty genes of Table S6, or, preferably, thirty to forty genes of Table S6.

The prognostic set, preferably about ten or about twenty or about thirty genes, may be selected from the first about forty, or about thirty, or about twenty genes of Table S6. About ten genes may be selected from the first about fifteen genes of Table S6. The about ten genes may be the first ten genes of Table S6.

The prognostic set may comprise, or consist essentially of, about forty or about thirty or about twenty or about ten genes selected from the group consisting of the first about forty or about thirty or about twenty or about ten genes of the Positive genes of Table S6 and, optionally, one or both Negative Genes of Table S6. The prognostic set may comprise, or consist of, about thirty genes selected from the group consisting of the first about thirty or about forty Positive genes of Table S6 and, optionally, one or both Negative genes of Table S6.

The number of genes in the prognostic set that are in common with the U133A microarray is preferably limited as described above.

The term ‘about’ preferably means the number of genes stated plus or minus the greater of: 10% of the number of genes stated or one gene.

The provision of the prognostic set allows diagnostic tools, e.g. nucleic acid microarrays to be custom made and used to predict, diagnose or subtype tumours. Further, such diagnostic tools may be used in conjunction with a computer which is programmed to determine the expression profile obtained using the diagnostic tool (e.g. microarray) and compare it, as discussed above, to a “standard” expression profile or a database of expression profiles of ‘known’ prognosis. In doing so, the computer not only provides the user with information which may be used diagnose the presence or type of a tumour in a patient, but at the same time, the computer obtains a further expression profile by which to determine the ‘standard’ expression profile and so can update its own database.

Thus, the invention allows, for the first time, specialized chips (microarrays) to be made containing probes corresponding to the prognostic set. The exact physical structure of the array may vary and range from oligonucleotide probes attached to a 2-dimensional solid substrate to free-floating probes which have been individually “tagged” with a unique label, e.g. “bar code”.

Querying a database of expression profiles with known prognosis can be done in a direct or indirect manner. The “direct” manner is where the patient's expression profile is directly compared to other individual expression profiles in the database to determine which profile (and hence which prognosis) delivers the best match. Alternatively, the querying may be done more “indirectly”, for example, the patient expression profile could be compared against simply the “standard” profile in the database for a particular prognostic assignment e.g. ‘bad’, or a prognostic value or range of values, preferably from the NPI e.g. high NPI. The advantage of the indirect approach is that the “standard” profiles, because they represent the aggregate of many individual profiles, will be much less data intensive and may be stored on a relatively inexpensive data carrier or other memory device (e.g. computer system) which may then form part of the kit (i.e. in association with the microarrays) in accordance with the present invention.

In the direct approach, it is likely that the data carrier will be of a much larger scale (e.g. a computer server), as many individual profiles will have to be stored.

By comparing the patient expression profile to the standard profile (indirect approach) and the pre-determined statistical variation in the population, it will also be possible to deliver a “confidence value” as to how closely the patient expression profile matches the “standard” canonical profile, as discussed above. This value will provide the clinician with valuable information on the trustworthiness of the prognosis, and, for example, whether or not the analysis should be repeated.

As mentioned above, it is also possible to store the patient expression profiles on the database, and these may be used at any time to update the database.

In a sixth aspect, the present invention provides a method for identifying a set of genes that are differentially expressed within a group of tumours, the method including providing an expression profile from each of a plurality of tumours of the group, classifying the profiles according to molecular subtype of tumour, and analysing expression profiles within a subtype to identify the set of genes, wherein the genes are differentially expressed within that subtype.

This method differs from the method of van't Veer et al. (10) in that the initial selection of sporadic, lymph node negative breast tumours in van't Veer et al. involved subtyping by clinical assessment, rather than subtyping at the molecular level.

Of course, this aspect and the following aspects of the invention are closely related to the preceding aspects. Preferred features disclosed for the preceding aspects may therefore be applied also to this aspect and the following aspects, unless the context clearly requires otherwise.

In the context of the sixth, seventh and eighth aspects of the invention, the term “expression profile” is not limited to the genes of the prognostic set. Rather, it refers generally to the expression levels of genes in the tumours of the group, including (but not necessarily only) the expression levels of genes that are differentially expressed within a molecular subtype.

Differential expression of the set of genes derived by the sixth aspect of the invention (hereinafter ‘the discriminating set’) may be indicative or characteristic of a particular phenotype or genotype for tumours of the group. The method preferably includes the step of correlating the differential expression of the discriminating set to a particular phenotype and/or genotype. The expression profile of the discriminating set in a number of samples of differing but known phenotype and/or genotype may be determined to establish a correlation between a particular gene expression profile of the discriminating set and a particular phenotype and/or genotype.

The differential expression may be characteristic of a clinical parameter or medical class assigned to the tumour as part of therapy or diagnosis of the patient with the tumour e.g. a measure of prognosis, such as an NPI value or NPI class. The differential expression of the discriminating set may allow a tumour sample to be assigned to one of at least two different genotypic or phenotypic classes.

The method of the sixth aspect of the invention may further include steps to assign a class to a tumour sample from a patient, wherein differential expression of genes of the discriminating set are characteristic of the class, the steps including providing expression levels in the sample of the discriminating set, and assigning a class to the tumour based on the expression levels.

The step of assigning the class may comprise the use of a statistical technique such as, but not limited to, Weighted Voting, Support Vector Machines or Hierarchical Clustering, as discussed previously. Preferably, the method includes the step of identifying the molecular subtype of the tumour sample, and using the discriminating set specific to the subtype.

Additionally or alternatively, the method of the sixth aspect of the invention may include the steps of determining the expression levels of the discriminating set in a tumour sample, determining an expression profile from the expression levels and adding the profile to a database. Preferably, the molecular subtype of the tumour sample is also identified, and preferably added to the database.

Standard profiles, characteristic of a particular class may be derived from at least two expression profiles of known class, wherein the expression profiles are derived from genes of the discriminating set. The standard profile is preferably specific to class and molecular subtype. Additionally or alternatively, expression profiles of known class (and, optionally, subtype) are added to the database.

Additionally, or alternatively, the method of the sixth aspect may further include steps to check for a change in class of the tumour during treatment. In one embodiment, expression profiles are provided from the tumour at different stages of treatment (e.g. start of treatment and end of treatment) and compared to determine a change in class, wherein the expression profiles are derived from the expression levels of genes of the discriminating set. The expression profiles are preferably compared to standard and/or known profiles to determine the class.

The classification according to molecular subtype is preferably performed using techniques, such as histopathological (e.g. immunological) techniques or gene expression techniques, that directly measure levels of gene expression products in tumour samples. Gene expression techniques are most preferred. However, clinical techniques that are capable of accurately discriminating between molecular subtypes may also be used.

The tumours are preferably breast tumours and the molecular subtype preferably corresponds to the ER (Estrogen Receptor) status of the tumour (e.g. ER+). However, the method may be applied to other groups of tumours (e.g. lung tumours, ovarian tumours and lymphomas) and/or other molecular subtypes (e.g. germinal centre-like and activated B-cell like in diffuse large B-cell lymphomas). Preferably the analysis performed on the class of expression profiles to determine the differentially expressed genes includes significant analysis of microarrays (SAM, ref. 12), which identifies genes whose expression levels vary significantly between samples under comparison. Preferably, the analysis involves statistical analysis, for example using Weighted Voting, Support Vector Machines and/or Hierarchical clustering (see later for an explanation of these techniques).

In a seventh aspect of the invention, there is provided the set of genes derived by the sixth aspect of the invention.

In an eighth aspect of the invention, there is provided the use of the discriminating set in assigning a tumour sample to a particular class.

Aspects and embodiments of the present invention will now be illustrated, by way of example, with reference to the accompanying figures. Further aspects and embodiments will be apparent to those skilled in the art. All documents mentioned in this text are incorporated herein by reference.

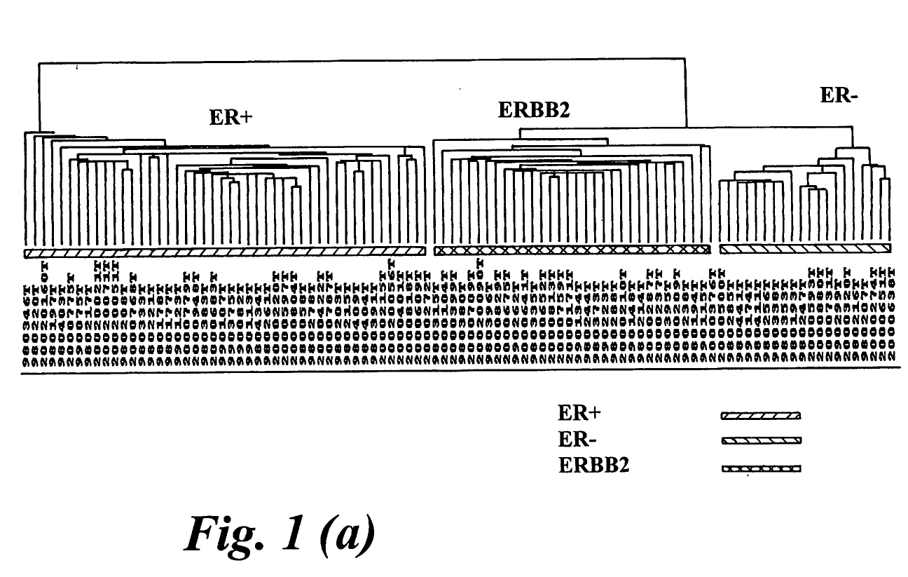

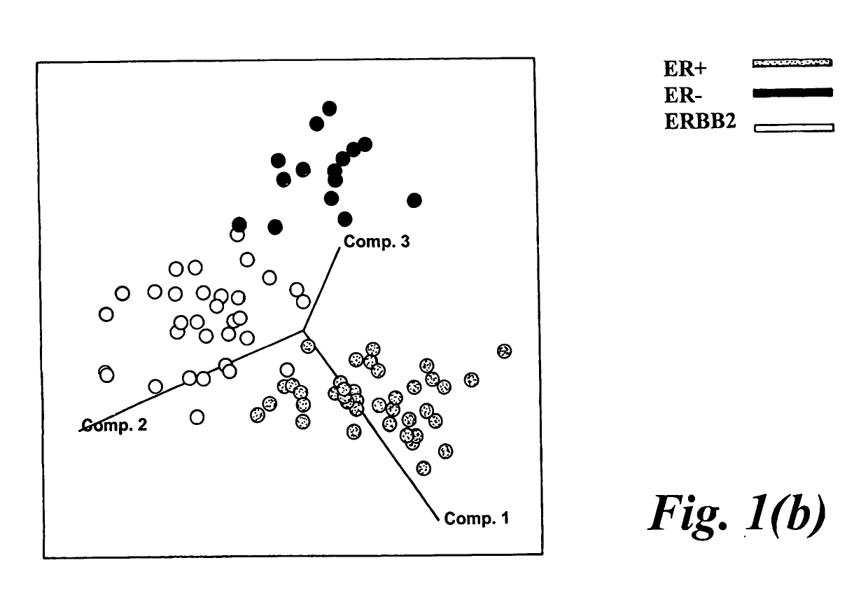

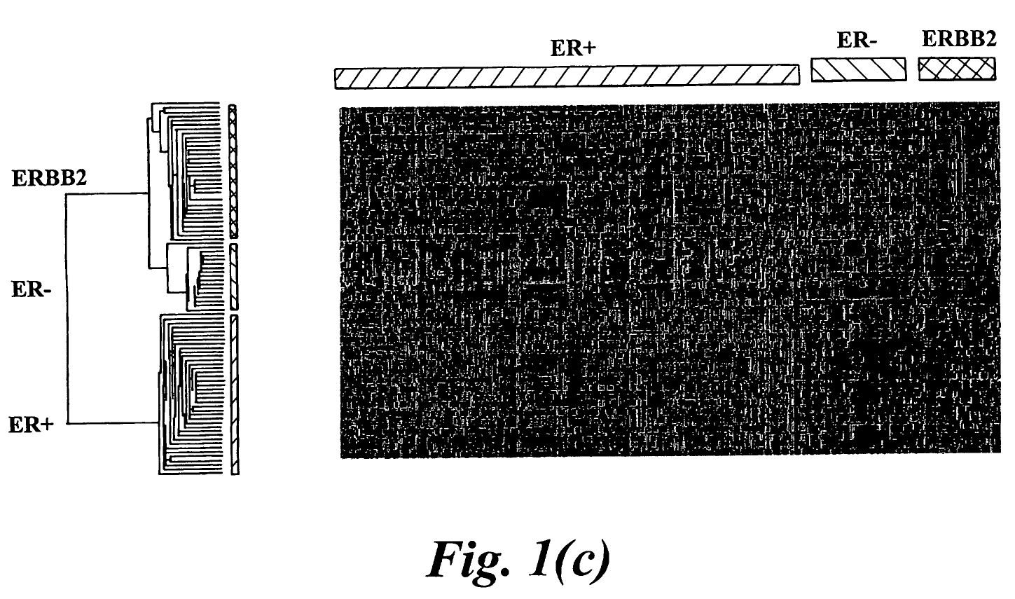

FIG. 1 shows clustering of sporadic breast tumors by global expression profiles a) Unsupervised hierarchical clustering of 98 breast tumors using the top 376 genes exhibiting the highest variation in gene expression, b) Principal component analysis (PCA) using the 376 gene set. Similar molecular groupings are observed as in a), a) Hierarchical clustering of samples using the SAM-409 gene set, which consists of genes that are significantly regulated between tumor subtypes. Approximately two-thirds of the genes in the SAM-409 gene set exhibit increased expression in ER+ tumors.

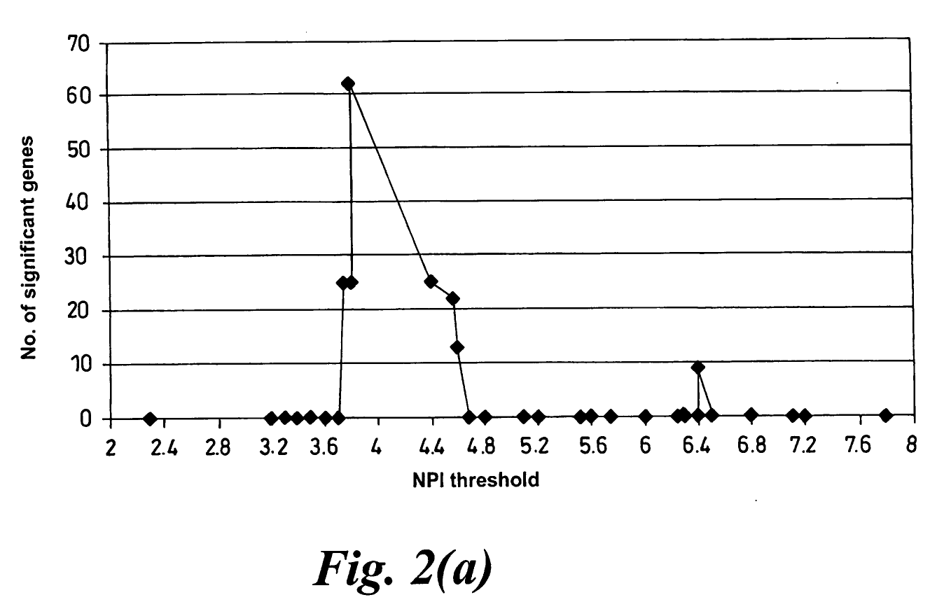

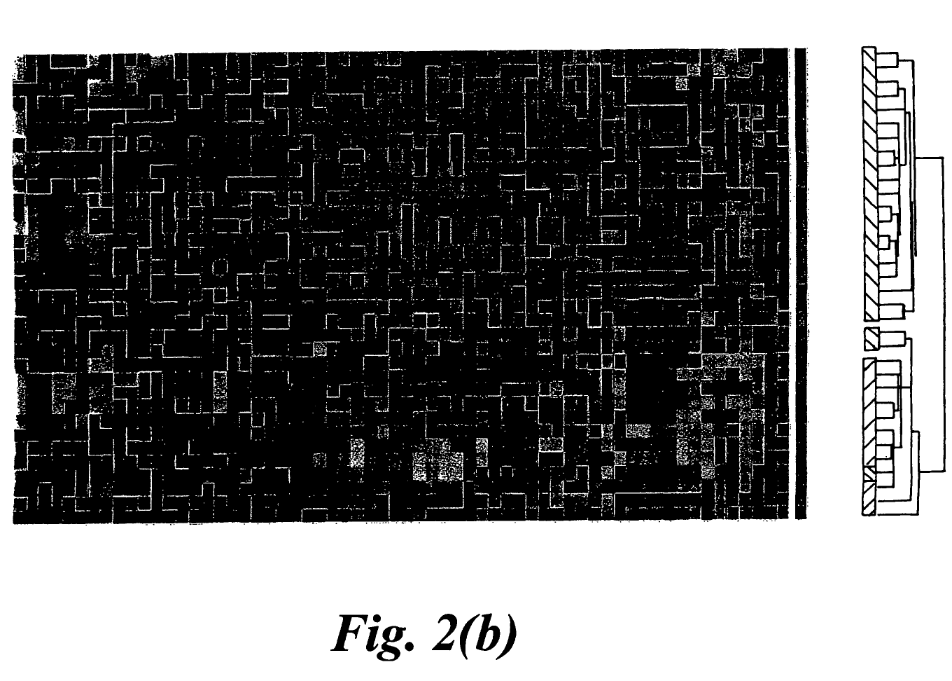

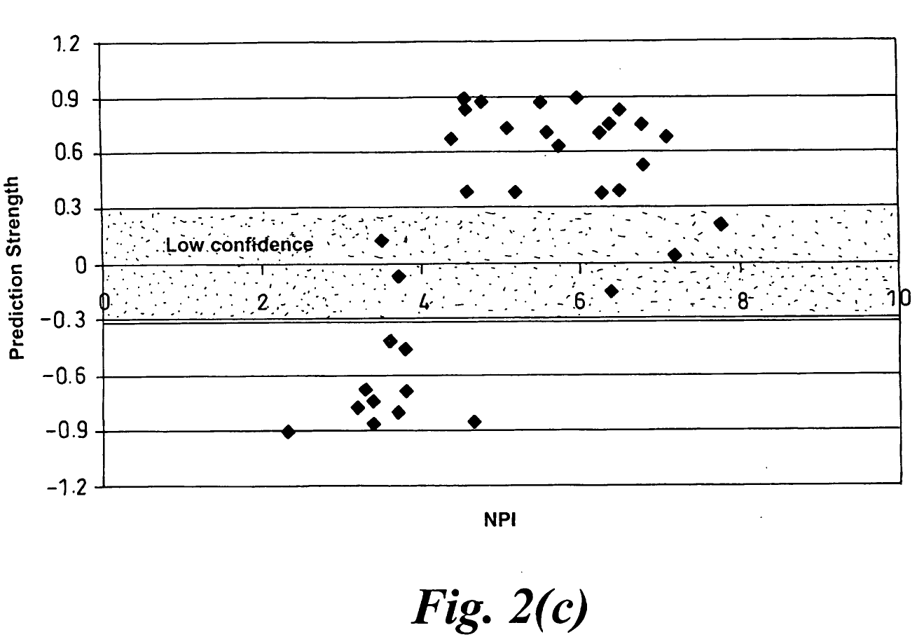

FIG. 2 shows identification of an Expression Signature Correlated to the NPI (NPI-ES):

a) Determination of differentially expressed genes using a moving NPI threshold. Genes (y-axis) exhibiting significant differential expression were identified at each threshold value (x-axis). Using a threshold of 4 delivers the highest number of differentially regulated genes,

b) Hierarchical clustering of ER+ samples using the NPI-ES. The red bar indicates samples of low NPI (<4); while the blue bar indicates samples of high NPI

c) Classification and prediction confidence of ER+ tumor samples using the NPI-ES. Samples are sorted by their NPI value (X-axis). Weighted voting was used to classify the samples and the prediction strengths of each sample (Y-axis) calculated based upon Golub et al. (13). Sample classifications with a prediction strength of <0.3 are considered ‘uncertain’ or ‘low-confidence’ (grey area).

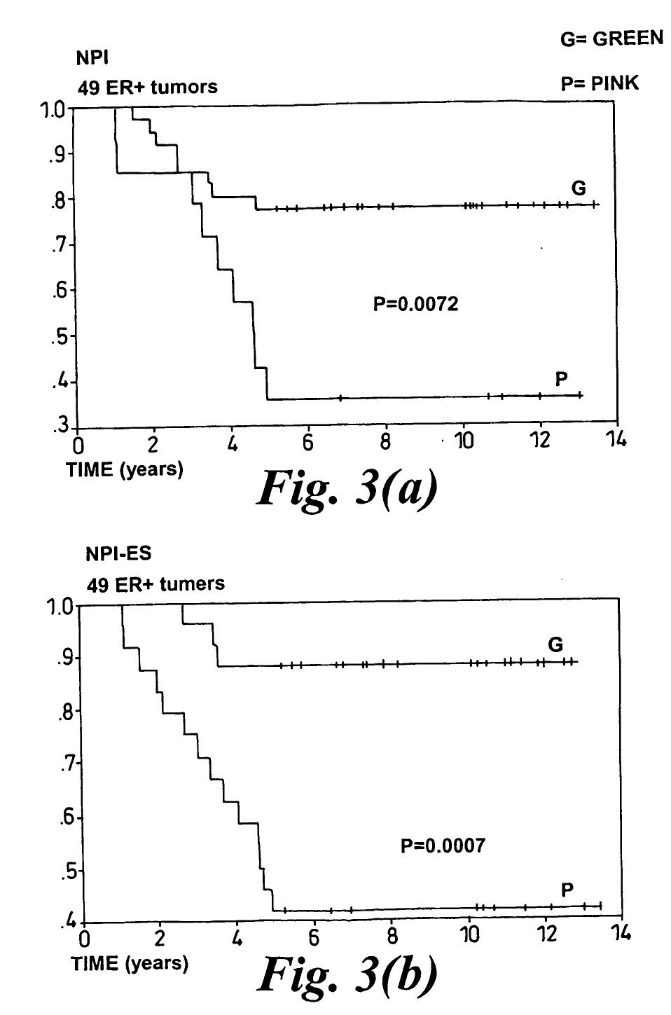

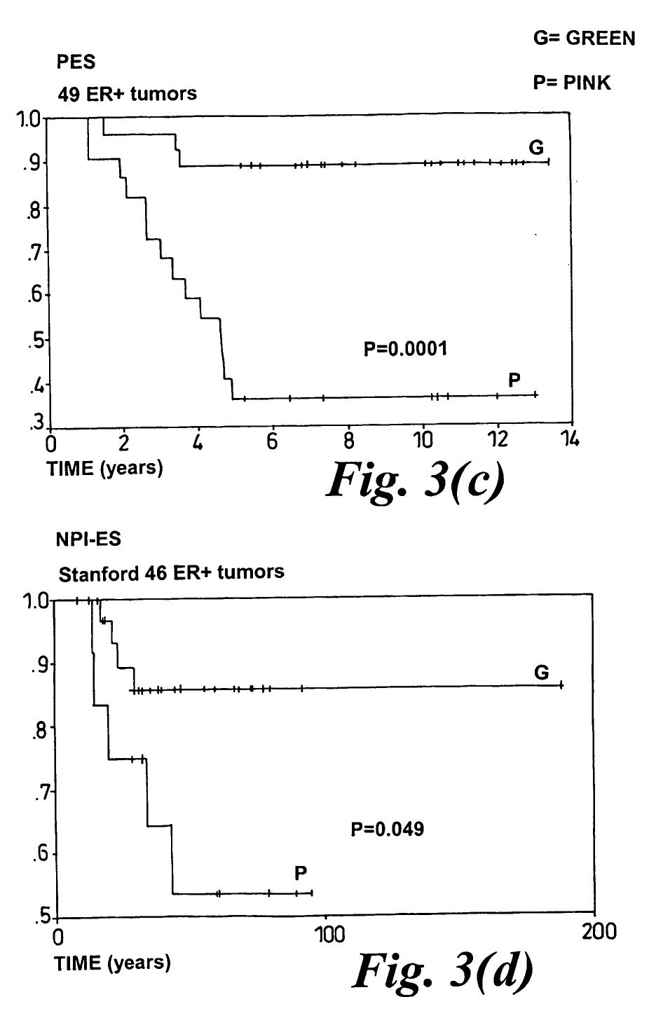

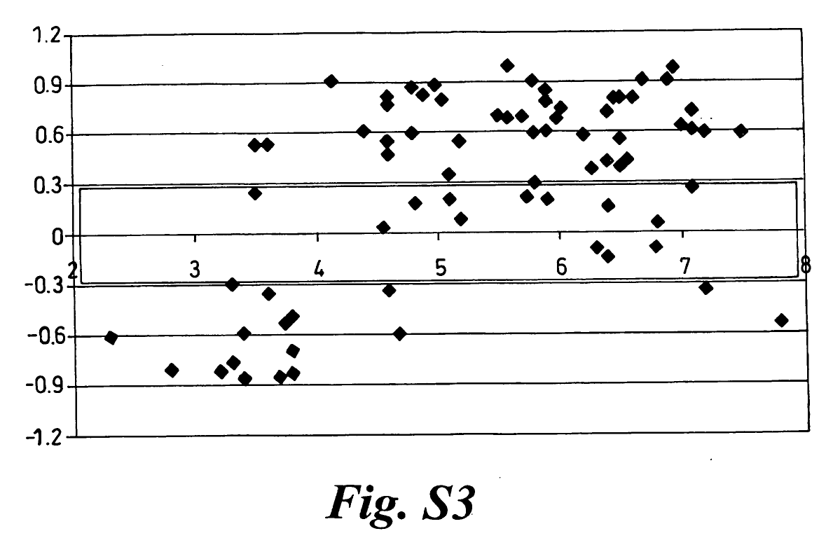

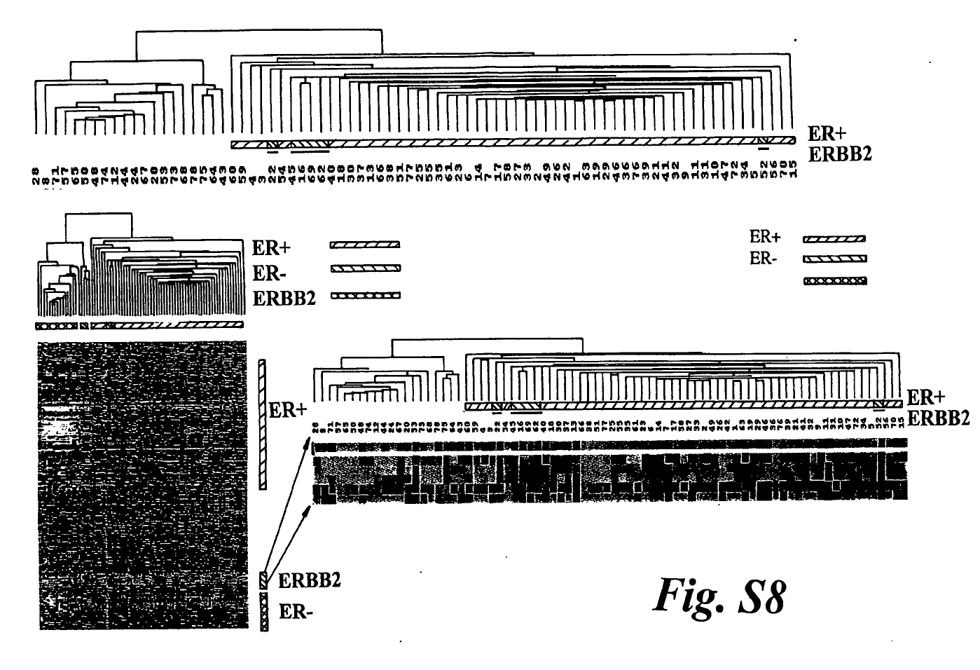

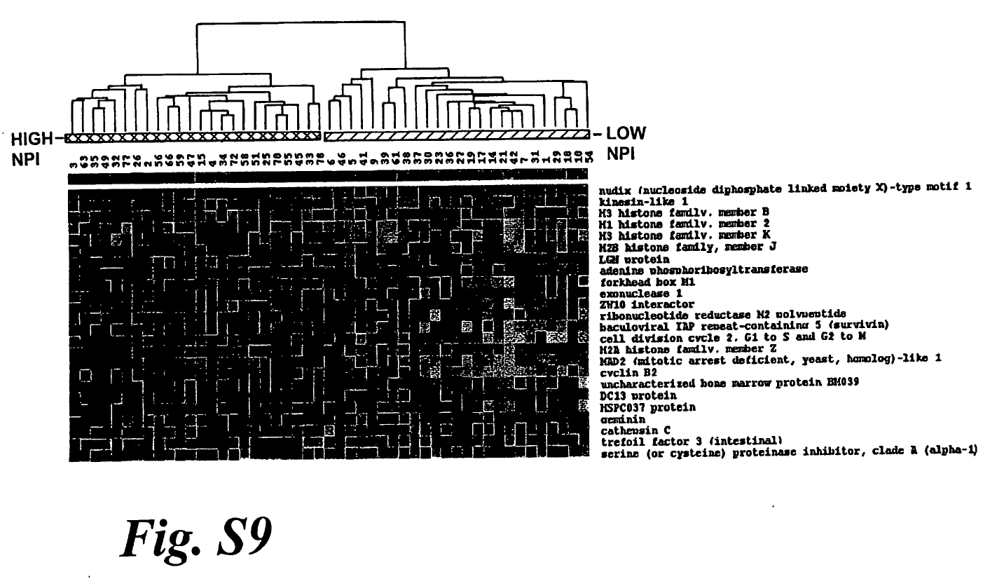

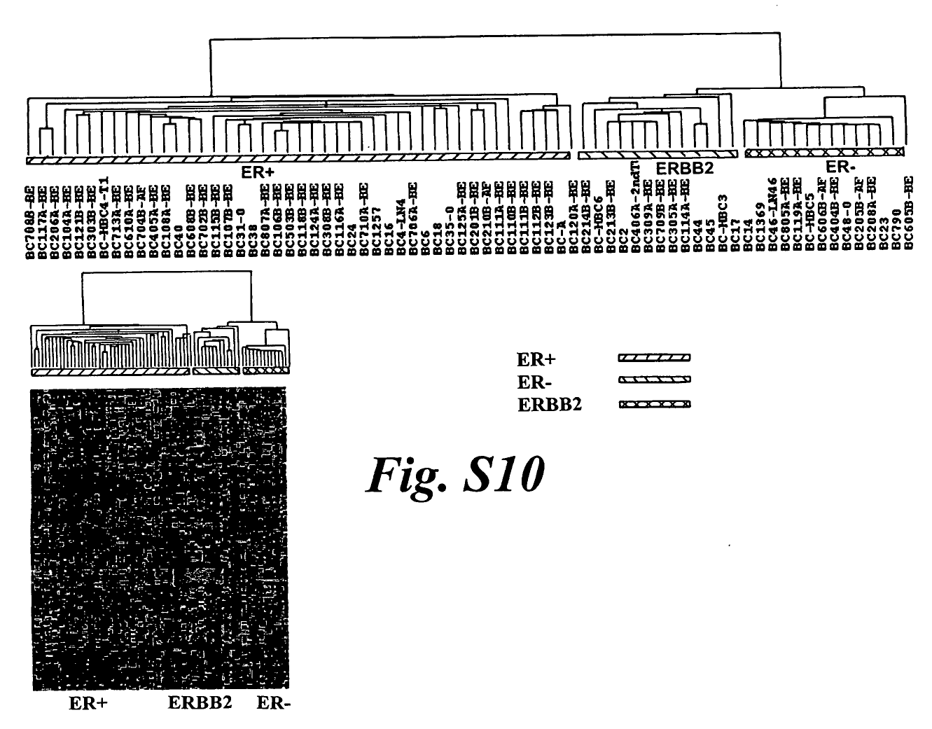

FIG. 3 shows KM Survival Analysis Comparing the Prognostic Strengths of Different Classification Schemes on ER+ Tumors. Green lines represent (a) low NPI, (b) low NPIES expression levels, or (c) low ‘prognosis’ signature (PES) expression levels, while pink lines represent high levels. (a) 49 Rosetta ER+Tumors stratified by classical NPI into ‘good’ prognosis (NPI<3.4) (35 tumors) and ‘moderate’ prognosis (NPI>3.4) (14 tumors) groups. (b) The same 49 Rosetta ER+ Tumors stratified by NPI-ES into groups expressing high (24 tumors) vs low levels of the NPI-ES (25 tumors). (c) The same 49 Rosetta ER+Tumors stratified by the 70-gene ‘prognosis’ signature into ‘good prognosis’ group (27 tumors) vs ‘poor prognosis’ group (22 tumors) respectively (d) The 46 Stanford ER+ Tumors stratified by NPI-ES into groups expressing high (13 tumors) vs low (33 tumors) levels of the NPI-ES.

FIG. S3 shows classification and prediction confidence of tumor samples using the 44-gene set based on all tumors regardless of subtype.

FIG. S8 shows hierarchical clustering of gene expression data from Rosetta data set. Top) Dendrogram displaying the similarities between tumors The color-coded bar indicated the subtype to the corresponding gene signature. Left) The full cluster of 276 genes with three distinct gene clusters. Note that some ERBB2 tumors appeared to segregate with ER+ tumors (red bar), but were identified as ERBB2+upon close inspection of expression of ERBB2+-related genes (zoom up of clustergram). This is due to the Rosetta microarray possessing a much higher number of genes related to the ER+ subtype than the ERBB2 subtype.

FIG. S9 shows hierarchical clustering of Rosetta ER+ samples (49) based upon the expression level of the NPI-ES (46 matches found in Rosetta data out of 62 genes). The color bar is as defined in FIG. 2b.

FIG. S10 shows hierarchical clustering of Stanford breast tumors. Top) Dendrogram displaying the similarities between tumors. The color-coded bar indicated the subtype to the corresponding gene signature. Left) The full cluster of 136 genes with three distinct gene cluster.

FIG. S11 shows hierarchical clustering of Stanford 46 ER+ samples using NPI-ES (31 matches out of 62 genes). The color bar is defined as FIG. 2b).

FIG. S12 shows the relationship between NPI-ES Expression and NPI Status in the ER− and ERBB2+Molecular Subtypes. The NPI status of ER− and ERBB2 tumors is in general higher than ER+ tumors. Unlike the case for ER+ tumors, we were unable to identify by SAM genes that were differentially regulated in high vs low NPI tumors for the ER− and ERBB2+ subtypes. Also, NPI-ES does not appear to be correlated as well to NPI values associated with the other molecular subtypes.

FIG. S13 shows 20 pairs of samples, obtained ‘Before’ and ‘After’ 14 weeks doxorubicin treatment (Perou et al., 2000). Of the 20 ‘Before’ samples, 10 samples exhibited high levels of NPI-ES expression (H), and 10 exhibited low levels of expression (L). Of the former 10 samples, 6 retained high levels of expression after chemotherapy (H->H, depicted in Red), while 4 exhibited low levels of expression after treatment (H->L, depicted in yellow).

FIG. S14 shows a Kaplan-Meier Relapse-free survival analysis curve using the patients that contributed the 20 samples of FIG. S13.

### MATERIALS AND METHODS

Breast Tissues and Clinical Information

Human breast tissues were obtained from the NCC Tissue Repository, after appropriate approvals from the NCC Repository and Ethics Committees. Histological confirmation of tumour status and Estrogen Receptor (ER) and ERBB2 immunohistochemical status were provided by the Dept of Pathology at Singapore General Hospital (see Supplementary Information for clinical information). Samples contained at least 50% tumour content. NPI status was calculated as follows: tumour size (cm)*0.2+grade+lymph node pts (negative nodes=1 point; positive nodes, 1 to 3 positive=2 points; positive nodes, 4 or more=3 points). As tumour size in the Stanford data set was defined using the CAT system, we assigned an approximate value for each CAT grade (i.e., T1=2 cm, T2=3.5, T3=5, T4=3.5).

Sample Preparation and Microarray Hybridization

RNA was extracted from tissues using Trizol reagent and processed for Affymetrix Genechip hybridizations using U133A Genechips according to the manufacturer's instructions.

Data Processing and Analysis

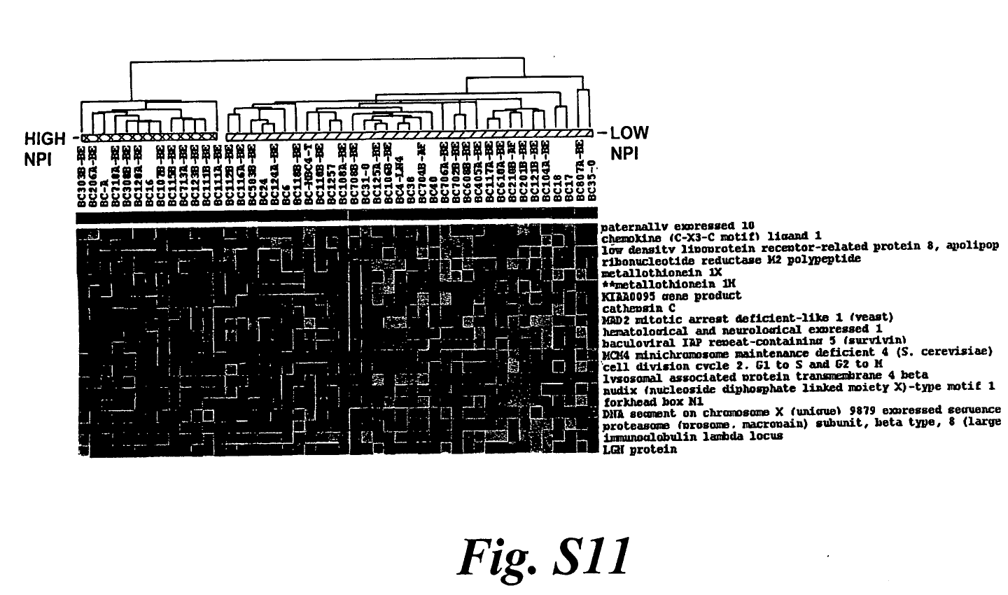

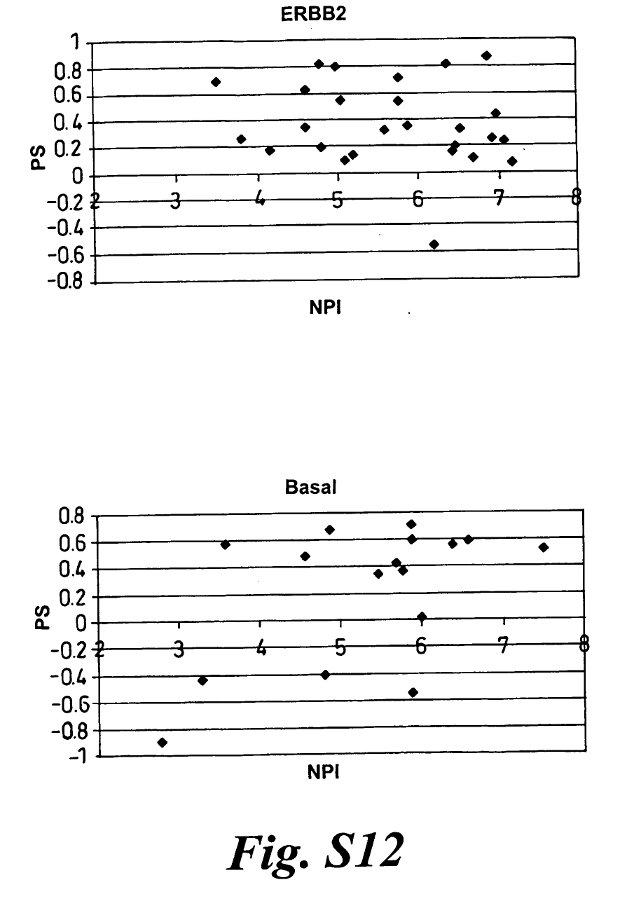

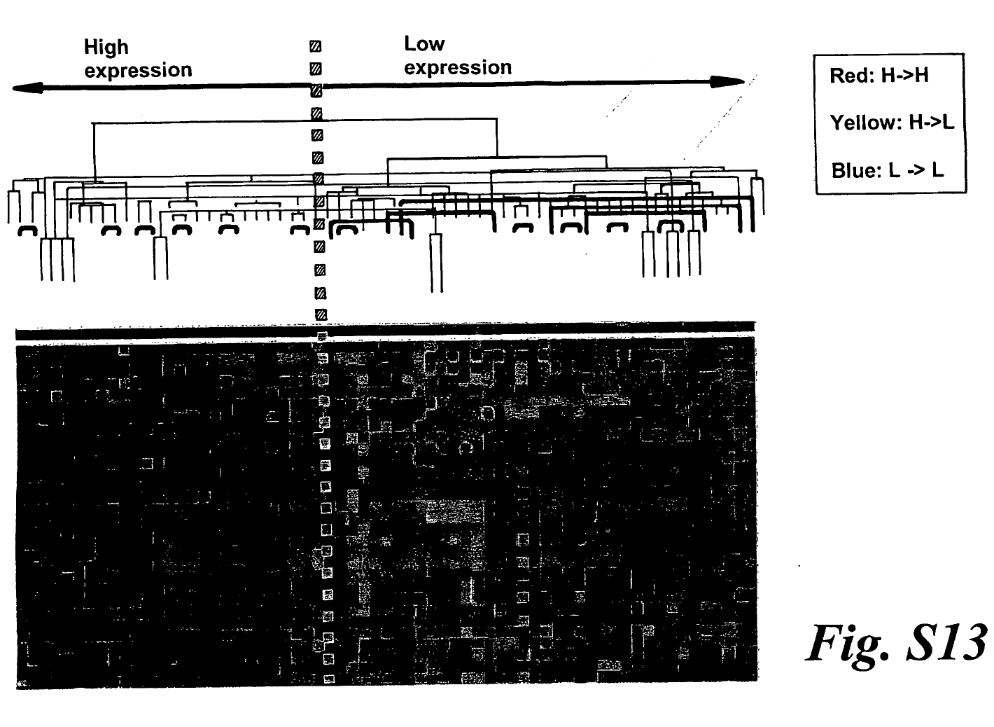

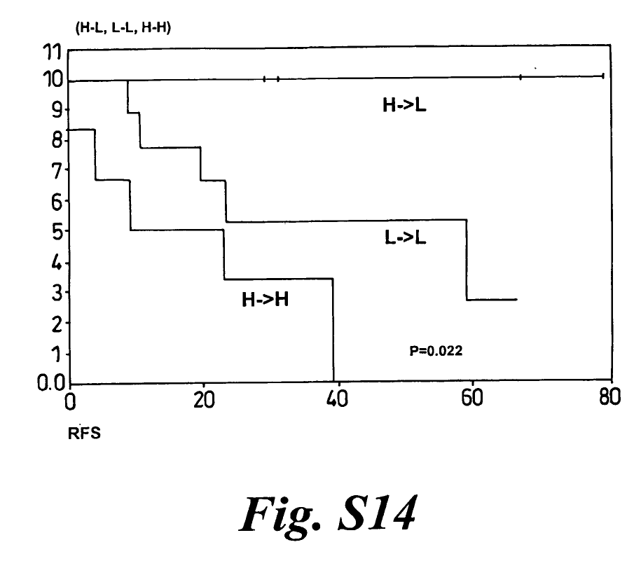

Raw Genechip scans were quality controlled using Genedata Refiner and filtered by removing genes whose expression was absent in all samples (i.e. ‘A’ calls). Expression values were subjected to a log2 transformation, and normalized by median centering all remaining genes by each sample. Data analysis was performed using Genedata Expressionist or conventional spreadsheet applications. The unsupervised dataset (FIG. 1, a-b) contains genes exhibiting a standard deviation (SD) of >1.5 across all well-measured samples. Minor variations of the variation filter used for gene selection also yielded very similar results (P. Tan, unpublished data). Duplicate probes for the same gene were removed from analysis, leaving one probe per gene. Average-linkage hierarchical clustering was performed using CLUSTER and displayed by using TREEVIEW. Significance Analysis of Microarrays (SAM) (12) was implemented to identify differentially regulated genes. ‘False discovery rates’ were 0.1% for FIG. 1c and 15% for FIG. 2. Weighted Voting (WV), Leave-one-out cross validation (LOOCV) assays, and prediction strengths (PS) were calculated as in Golub et al., (13) (Supplementary Information). Kaplan-Meier survival curves were created using SPSS, and log-rank tests used to calculate the statistical significance of differences between survival curves. Statistical associations between gene expression and clinical variables were determined by chi-square analysis.

Descriptions of Weighted Voting (WV) and Leave-One-Out Cross Validation (LOOCV) Assays

Weighted Voting (WV): The weighted voting algorithm utilizes a signal-to-noise (S2N) metric to perform binary classifications. Each gene belonging to a predictor set is assigned a ‘vote’, expressed as the weighted difference between the gene expression level in the sample to be classified and the average class mean expression level. Weighting is determined using the correlation metric:

$$P ( g , c ) = \frac{{μ}_{1} - {μ}_{2}}{{σ}_{1} + {σ}_{2}}$$

(μ and θ denotes means and standard deviations of expression levels of the gene in each of the two classes). The ultimate vote for a particular class assignment is computed by summing all weighted votes made by each gene used in the class discrimination. The “prediction strength” (PS) is defined as:

$$PS = \frac{{V}_{WIN} - {V}_{LOSE}}{{V}_{WIN} + {V}_{LOSE}}$$

where V_{WIN} and V_{LOSE} are the vote totals for the winning and losing classes, respectively. PS reflects the relative margin of victory and hence provides a quantitative reflection of prediction certainty.

Leave-One-Out Cross Validation (LOOCV): We used a standard leave-one-out crossvalidation (LOOCV) approach to assess classification accuracy in the training set. In LOOCV, one sample in the training set is initially ‘left out’, and the classifier operations (e.g. gene selection and classifier training) are performed on the remaining samples. The ‘left out’ sample is then classified using the trained algorithm, and this process is then repeated for all samples in the training set.

Results and Discussion

Defining Molecular Subtypes of Breast Cancer Using Unsupervised Clustering

It has been proposed that a significant proportion or one intrinsic gene expression variation in breast cancer can be attributed to different tumours belonging to distinct ‘molecular subtypes’ (e.g. ER+ and ER− tumours) (8-9, 14). In an initial analysis where tumours were treated irrespective of subtype, we could not convincingly identify an expression signature correlated to the NPI. We hypothesized that this might be due to dramatic differences in gene expression between subtypes (inter-subtype differences) potentially obscuring more subtle patterns of variation within subtypes (intra-subtype differences). To circumvent this problem, we implemented a methodology where each molecular subtype was treated as an independent data set. Briefly, a variety of unsupervised clustering techniques were first used to broadly segregate a set of breast tumour expression profiles according to their respective ‘molecular subtype’ categories. Second, tumours within each subtype were then independently analyzed to define expression signatures that might be correlated to the NPI or its constituent elements.

Using Affymetrix U133A Genechips, we generated expression profiles for 98 sporadic breast tumours derived from our local predominantly Chinese patient population. After data normalization and pre-processing, we applied a standard deviation filter to identify a 367 gene set exhibiting a high degree of gene expression variation across the tumour series, and used this gene set to group the tumour expression profiles on the basis of their overall similarity using unsupervised hierarchical clustering. The breast tumours self-segregated into three major subgroups, referred to as ER+, ER−, and ERBB2+respectively (FIG. 1a). This segregation pattern was confirmed using principal components analysis (PCA), an independent analytical technique (FIG. 1b), which delivered highly similar results. To robustly identify these groupings, we used SAM (12) to identify genes that were differentially expressed between the subtypes. At a FDR (‘False Discovery Rate’) of 0.1%, we identified 409 genes that were significantly regulated in a subtype-specific manner (FIG. 1c).

The list of Table S5 represents the top 50 genes identified by SAM to be significantly regulated in each molecular subtype (ER+, ER−, ERBB2+). The genes are ranked by their S2N correlation ratio, which reflects the extent of the expression perturbation observed among different groups.

There is good overlap between these genes and similar lists reported by other studies (ref. 8-11)

Approximately 69% of the 409 gene set exhibited increased expression in the ER+ subgroup, including the estrogen receptor gene ESR1 and estrogen-regulated genes such as LIV1, TFF1, and MYB (Supplementary Information). In agreement with other studies, high expression levels of GATA3, HNF3a, Annexin A9, and XBP1, were also observed in this subtype (8-9, 11). The ER− subgroup was associated with high expression of basal mammary epithelia markers (keratin 5 and 17), the basement membrane protein ladinin 1, the serine protease KLK5, which has been associated with poor disease prognosis, (15), and the serine protease inhibitor maspin, a tamoxifen-inducible gene that has been previously reported to be expressed in an inverse fashion to ER (16). Finally, the ERBB2+ subtype was associated with high expression levels of the ERBB2 receptor and other genes physically linked to the 17q locus, such as GRB7 and PMNT (14), suggesting the presence of DNA amplification. However, the majority of genes exhibiting increased expression specifically in the ERBB2+ subtype are not confined to the 17q locus but are found throughout the genome, such as members of the S100 calcium-binding family (S100A8, A9). Taken collectively, our results validate and confirm previous reports that the majority of breast tumours can indeed be subdivided into distinct molecular subtypes on the basis of their global gene expression profiles.

Identification of a Prognostic Set Correlated to the NPI in ER+ Tumours

We focused on 34 tumours belonging to the ER+ molecular subtype and attempted to identify genes within this subtype whose expression might be correlated to NPI status. Classically, breast cancer patients are typically stratified by the NPI into 3 major groups—‘good’ prognosis (NPI<3.4), ‘moderate’ prognosis (NPI 3.4-5.4), and ‘poor’ prognosis (NPI>5.4) (2). Possibly reflecting the effects of variability across different scoring pathologists, other studies have proposed slightly different values for the cut-off values defining these groups (17). To avoid any potential bias in determining the appropriate NPI cut-off value, we conducted a moving threshold analysis where the ER+ tumours were divided into a series of binary groups by a NPI threshold that was steadily increased from 2.3-7.8. At each threshold value, genes exhibiting significant variation in expression between the two groups were identified. We found that using an NPI cut-off value of 3.8 to 4.6 yielded a gene set of 62 differentially expressed genes (FIG. 2a), the majority of which exhibited increased expression in the ER+ samples with a high NPI (FIG. 2b). We refer to this 62-member gene set as an ‘NPI Expression Signature’ or NPI-ES, shown in Table S6. The genes belonging to the NPI expression signature are associated with a wide variety of cellular functions implicated in oncogenesis, including DNA replication and cell division (APRT, MCM4, KNSL 1, CDC2), cellular signaling (chemokine ligand 1, Met, ShC), apoptosis (survivin, CD27 binding protein), and cellular adhesion (discs-large homolog 7, tetraspan 1). Of the individual NPI components (tumour size, tumour grade, lymph node status), tumour grade appears to represent the predominant contributor to the molecular makeup of the NPI-ES (Supplementary Information).

Classification of Tumours by the NPI-ES Defines Two Discrete Molecular Groups

One proposed advantage in the use of molecular profiles for tumour classification is the ability to mathematically quantify the confidence level of the classification (11), which is particularly important if the classification affects the subsequent course of treatment. In such a scenario, the treating physician can then weigh the confidence level of a prediction against the potential morbidity of a specific intervention. Notably, although the ER+ samples in our data set were associated with a continuous spectrum of classical NPI values (2 to 8), the clustering analysis using the NPI-ES appeared to separate the ER+ tumours into two apparently discrete groups (FIG. 2b), raising the possibility that samples exhibiting continuous values based upon histopathological parameters may be nevertheless separable into discrete categories at the molecular level.

To better define the ability of the NPI-ES to confidently discriminate between these two classes, we used Weighted Voting (13), a supervised learning algorithm, to distinguish between tumours exhibiting high and low expression of the NPI-ES, and tested the classification accuracy of the trained algorithm using an established leave-one-out cross validation (LOOCV) assay. In addition to classification accuracy, quantitative metrics (prediction strengths, PS) were also calculated as described in Golub et al., (13) to provide an assessment of the prediction confidence (FIG. 2c). The WV analysis revealed that the NPI-ES delivered a LOOCV classification accuracy of 91%, with 3 misclassifications. Of the 3 samples that were wrongly classified, 2 were associated with a low prediction strength (PS<0.3), and thus represent ‘low-confidence’ or ‘uncertain’ classifications. Indeed, of the 29 (out of 34) ER+ tumours associated with a ‘high-confidence’ classification (PS>0.3), only one sample was wrongly classified. These results suggest that the NPI-ES can be used to classify the majority of the ER+ tumours in our data set into discrete groups with high confidence.

Derivation of a NPI Expression Signature Using All Tumors, Regardless of Subtype

We defined the NPI-ES using a two-step methodology. Initially, unsupervised clustering was used to cluster tumors according to their respective ‘molecular subtype’ (i.e. ER+, ER−, ERBB2+). Tumors within each subtype were analyzed for expression signatures that might be correlated to the NPI. Here, we show that performing the first step (definition of distinct molecular subtypes) is important in the identification of the NPI-ES.

We assembled a data set consisting of all 79 tumors, regardless of molecular subtype, and performed a moving NPI threshold analysis to define an ‘appropriate’ NPI threshold, as above (see FIG. 2a). We found that using an NPI threshold of 4 yielded a total of 44 differentially expressed genes: Of this 44 gene set, 16 (35%) also belong to the NPI-ES (which was derived from ER+ samples).

We used Weighted Voting (WV) and cross-validation (LOOCV) assays to assess the ability of this 44 gene set to confidently classify the tumor samples into discrete groups. As can be seen in FIG. S3, the number of low-confidence (PS<0.3, red area) samples, as well as the misclassification rate (9% for the 44 gene set) are both significantly increased compared to FIG. 2c. This result indicates that the 44-gene set, based upon all 79 tumors, is less effective in predicting the NPI status of a tumor than the NPI-ES on ER+ tumors.

In FIG. S3 Samples are sorted by their NPI value (X-axis). Weighted voting was used to classify the samples and the prediction strengths of each sample (Y-axis) calculated based upon Golub et al., (13). Sample classifications with a prediction strength of <0.3 are considered ‘uncertain’ or ‘low confidence’ (grey area). A higher number of ‘uncertain’ (low PS) samples and misclassified samples are observed compared to FIG. 2c.

The 44 gene set derived from all tumors regardless of subtype is also not as effective as the NPI-ES at predicting NPI status in an independent data set. Using the Rosetta data set as a blinded test set, we applied the 44 gene set to the 49 ER+ tumors found in the Rosetta data set, and used Student's t-test to determine the significance of association between a ER+ tumors expressing high levels of the 44 gene set and possessing a high NPI. We obtained a p-value of 0.29 for the 44 gene set, which was much less significant compared to a p-value of 0.0004 for the NPI-ES.

Interestingly, the NPI-ES, despite being derived from an analysis of ER+ tumors, outperforms the 44 gene set even when applied across all 78 tumors in the Rosetta data set. To illustrate this, the 78 Rosetta tumors were divided into two groups of NPI<3.4 (good prognosis) and >3.4 respectively (moderate prognosis). Weighted voting was then used to classify the Rosetta tumors by the NPI-ES or the 44 gene set. As can be seen in Table S3, the NPI-ES delivered a classification accuracy of 80%, compared to the 44 gene set which delivered a 70% classification accuracy.

Genes Associated with Histological Grade (1 & 2 vs. 3)

Since the classical NPI is a composite metric derived from tumor grade, tumor size, and lymph node status, we defined the contributions made by each of these individual elements to the molecular makeup of the NPI-ES. Using SAM to identify genes correlated to each of the three histopathological variables, we were unable to convincingly identify genes whose expression was significantly correlated to either tumor size or lymph node status. In contrast, in the case of histological grade, a significant number of genes were found to be differentially expressed between grade 1 or 2 and grade 3 tumors, and the genes in this grade-correlated gene set exhibited substantial overlap (66%) with the NPI-ES (Table S6). These results suggest that tumors exhibiting different histological grades may be biologically distinct, and that tumor grade is a key contributor to the NPI expression signature, with the remaining two parameters (tumor size and lymph node status) delivering comparatively lesser contributions.

Application of the NPI-ES Across Multiple Independent Breast Cancer Expression Data Sets

To test the ability of the NPI-ES to predict both NPI status and disease prognosis in a series of blind ‘test sets’, we used two independent breast cancer data sets that were publicly available. The first data set (referred to as the Rosetta data set) consists of 78 lymph-node negative breast tumours profiled using oligonucleotide-based microarrays, and also contains the duration of ‘disease free survival’ (DFS) (the time from initial tumour diagnosis to the appearance of a new distant metastasis) for each patient (10). Importantly, several studies have previously shown the NPI to be of prognostic value even in node-negative breast cancers (18, 19). The second data set consists of 78 breast carcinomas profiled using cDNA microarrays with overall patient survival information (referred to as the Stanford data set) (14). The availability of these data sets allowed us to independently test the predictive power of the NPI-ES, as the Rosetta and Stanford data sets are different from our data set in multiple ways, including I) patient population, II) sample handling protocols, III) scoring pathologist and IV) choice of array technology and probe sets (two-color in the Rosetta and Stanford data sets and single color in ours).

Rosetta Breast Cancer Data Set: Of the 409 genes identified by SAM analysis defining the ER+, ER−, and ERBB2+ subtypes, 276 genes (67%) were found on the Rosetta microarray. We applied this gene set to the 78 Rosetta tumour profiles and identified 49 tumours belonging to the ER+ molecular subtype determined that 0.46 out of 62 genes belonging to the NPIES were also present on the Rosetta microarray. Since the Rosetta data set is based upon a different array technology from ours, it is not possible to directly apply the trained Weighted Voting model developed on our data set to classify the Rosetta tumours.

However, following the strategy described in Ramaswamy et al., (20) for the comparison of gene sets across different array technologies, we used hierarchical clustering to group the 49 ER+Rosetta tumours using the overlapping NPI-ES set of 46 genes. The clustering analysis divided the 49 ER+ Rosetta tumours into 2 groups consisting of 24 and 25 tumours exhibiting ‘high’ and “low” expression levels of the NPI-ES respectively (see FIG. S9).

We compared the tumours in these two subgroups to determine if they were associated with differences in their NPI values. Using two distinct statistical approaches where the tumour NPI values were treated either as a continuous gradient (Student's T-test), or as two discrete groups (Chi-square analysis, using classical NPI cut-off value of 3.4), tumours exhibiting high expression of the NPI-ES consistently exhibited with a significantly higher NPI value compared to tumours expressing low levels of the NPI-ES (p=0.0004 for continuous analysis, p=0.0087 for binary analysis) (Table 1a). This analysis indicates that expression of the NPI-ES is significantly correlated with classical NPI status in ER+ tumours even in an independent data set generated by a different array technology.

To compare the prognostic power of the NPI-ES to the classical NPI system of staging, odds-ratio calculations were performed (Table 1b). Patients with ER+ tumours expressing high levels of the NPI-ES had an odds-ratio for distant metastases within five years of 10.3 (95% CI 2.4 to 44.0, p<0.001) compared to ER+ tumours expressing low levels of the NPI-ES. In comparison, patients with ER+ tumours with a classical NPI index of >3.4 (‘moderate’ prognosis) had a lower odds-ratio for distant metastases of 6.1 (95% CI 1.6-23.4, p=0.06) compared to ER+ tumours with a NPI index of <3.4 (‘good’ prognosis). We also compared the prognostic performance of the NPI-ES and NPI using Kaplan-Meier survival analysis (FIG. 3). In agreement with other studies, patients with tumours of low NPI (<3.4) exhibited better DFS as compared to patients of higher NPI (>3.4) (p=0.007, FIG. 3a). When this same population was restratified by the NPI-ES, patients with tumours exhibiting high expression of the NPI-ES exhibited better relapse-free survival (p=0.0007) compared to patients with tumours expressing low levels of the NPI-ES. Taken collectively, this data suggests that for ER+ tumours, the prognostic power of the NPI expression signature may outperform the classical NPI system of staging.

Stanford Data Set: A similar approach was used to test the NPI-ES on the Stanford data set (see FIG. S10). Of the SAM-409 gene set used to define the ER+, ER−, and ERBB2+subtypes, 136 genes were found on the Stanford microarray (http://genome-www5.stanford.edu/MicroArray/SMD/), and these genes were used to cluster the Stanford tumours to identify 46 tumours belonging to the ER+ molecular subtype (from 72 tumors after discarding the normal-like tumor subgroup of 6 tumors, which subgroup is likely to be due to the presence of contaminating non-malignant tissue).

These 46 tumours were then clustered (see FIG. S11) using the NPI-ES (31 matches on the Stanford microarray) into ‘high-NPI-ES’ (13 tumours) and ‘low-NPI-ES’ groups (33 tumours). Once again, Student's t-test revealed a significant association (p=0.001) between the high and low expressing NPI-ES subgroups and classical NPI status (Table 1a). In addition, a KM survival analysis also demonstrated a significant (p=0.0493) overall survival advantage in patients with low-NPI-ES expressing tumours compared to patients with high-NPI-ES expressing tumours (FIG. 3d).

Interestingly, there appears to be a strong correlation between ER+ tumours expressing high levels of the NPI-ES and the ‘Luminal C’ molecular subtype identified in Sorlie et al., (14), although none of the 62 genes belonging to the NPI-ES have been reported to be expressed in the latter. Interestingly, Sorlie et al., (ref. 14), previously reported the identification of a “Luminal C” subtype based upon an ‘intrinsic’ set of 500 genes. There appears to be a strong overlap (96%) between ‘Luminal C’ tumors and tumors expressing high levels of the NPI-ES, although, as mentioned above, none of the 62 genes belonging to the NPI-ES are found in this ‘intrinsic’ set. This is illustrated in Table S11.

The Prognostic Capacity of the NPI-ES is Comparable to a Previously Described “Prognosis Signature” for Breast Cancer

In the same study by Van Veer et al (10), the authors also identified a 70-gene ‘prognosis’ expression signature (PES) that predicted the DFS status of breast tumours. Interestingly, there is minimal overlap between the genes belonging to the NPI-ES and the PES, as only one gene is found in common between the two. To compare the prognostic performance of the NPI-ES and the PES on the Rosetta ER+ tumours, we used KM survival analysis to compare the DFS of patients stratified either by the NPI-ES (FIG. 3b) or the PES (FIG. 3c). A slightly better performance was observed with the PES (p=0.0001) compared to the NPI-ES (p=0.0007). The marginal improvement associated with the PES, however, is not unexpected since the identification of the PES was directly based upon the expression profiles and clinical information of these same tumours. As such, the Rosetta tumours are not ‘blinded’ to the PES, while in the case of the NPI-ES, the Rosetta tumours represent a true independent test set. Indeed, when the PES and NPI-ES were applied to the Stanford ER+ tumours, both molecular signatures delivered highly similar odds-ratios (3.9 for PES vs 4.17 for NPI-ES) for relapse within 5 years (Table 1c). Thus, these results suggest that the prognostic power of the NPI-ES and PES are relatively comparable.

Expression of the NPI-ES Molecular Signature Predicts Chemotherapy Response

In this analysis, we examined the expression of the NPI-ES molecular signature in paired breast tumor samples before and after chemotherapy, and correlated the expression of this signature to eventual clinical response.

A publicly available breast cancer data set (“Stanford”) was utilized, consisting of 20 pairs of samples, obtained ‘Before’ and ‘After’ 14 weeks doxorubicin treatment (8). Of the 62 genes found in the NPI-ES, 31 genes were also found on the Stanford microarray, and the expression of the 31 gene set was examined in the paired samples.

Of the 20 ‘Before’ samples, 10 samples exhibited high levels of NPI-ES expression (H), and 10 exhibited low levels of expression (L). As shown in FIG. S13, of the former 10 samples, 6 retained high levels of expression after chemotherapy (H->H, depicted in Red), while 4 exhibited low levels of expression after treatment (H->L, depicted in yellow). The number of deaths (after 5 years) was then tabulated for each group as shown in Table S12.

A Kaplan-Meier Relapse-free survival analysis was then performed, and is shown in FIG. S14. We found that the ‘H->L’ tumors had the best survival outcome (p=0.022) compared to the other groups, while ‘H->H tumors had the worse prognosis. This result suggests that down-regulation of the NPI-ES in high-expression NPI-ES tumors can be taken as a marker of chemotherapy response.

In summary, we have identified a 62-gene expression signature that can potentially function as a molecular surrogate for the NPI. Confidence in the reliability of the NPI-ES was obtained by showing that it could predict both NPI status and disease prognosis for two independent sets of tumours generated by different centers. One interesting concept emerging from this study is that samples exhibiting apparently continuous variables at the histopathological level may nevertheless be separable into discrete categories at the molecular level. This may address a major challenge in cancer histopathology, namely the difficulty of defining clinically appropriate cut-off values when the parameter being scored is of a continuous nature. We conclude by acknowledging that more work needs to be performed before the clinical utility of the NPI-ES can be fully assessed. First, the predictive power of the NPI-ES obviously needs to be tested against a much larger group of tumours.

Second, although we have demonstrated the applicability of the NPI-ES in the ER+ molecular subtype, expression of the NPI-ES does not appear to be correlated as well to NPI values associated with the other molecular subtypes (ER−, ERBB2+) (Supplementary Information).

Sample Data

Table S14 shows expression data for the prognostic set (or NPI-ES) of genes across samples of differing NPI value. The data are specific for the Affymetrix U133A genechip and have been through data preprocess. The gene expression profiles of the prognostic set can be used as training data to build a predictive model (e.g., WV and SVM), which then can assign the NPI class of an unknown tumour.

The data is tab delimited, and has the following format:

Columns:

1st Column: Probe_ID of Prognostic Set Genes

2nd Column: Gene Name

3rd and Other Columns: Gene Expression Data

Rows:

1st Row: Sample Ids (35 samples)

2nd Row: NPI Index.

3rd and Other Rows: Gene Expression Data

The gene expression data is derived as described in the ‘Sample Preparation and Microarray Hybridization’ and ‘Data Preprocessing’ (see Materials and Methods section). In particular, raw gene expression data values are calculated by the instrument used to measure the microarray (usually a microarray scanner, e.g. Affymetrix).

Table S15 shows the mean (μ) and standard deviation (σ) parameters for use in a Weighted Voting algorithm for each gene of the prognostic set in each class. These data could be used to assign the prognosis of an unknown breast tumour sample given a set of expression levels for genes of the prognostic set. The data is specific to Weighted Voting techniques applied to expression data from Affymetrix U133A genechip.

### REFERENCES

1. Elston, C. W. and I. O. Ellis. Pathological prognostic factors in breast cancer: I. The value of histological grade in breast cancer—Experience from a large study with long-term follow-up. Histopathology 19, 403-410, 1991.
2. Galea, M. H., R. W. Blamey, C. W. Elston, and I. O. Ellis. The Nottingham Prognostic Index in primary breast cancer. Breast Cancer Res Treat. 22, 207-219, 1992.
3. Ellis, I. O., M. Galea, N. Broughton, A. Locker, R. W. Blamey, and C. W. Elston.
   Pathological prognostic factors in breast cancer. II. Histological type. Relationship with survival in a large study with long-term follow-up. Histopathology 479-489, 1992.
4. Balslev, I., C. K. Axelsson, K. Zedeler, B. B. Ramussen, B. Carstensen, and H. T. Mouridsen. The Nottingham Prognostic Index applied to 9,419 patients from the studies of the Danish Breast Cancer Cooperative Group (DBCG). Breast Cancer Res. Treat. 32, 281-290, 1994.
5. Sauerbrei, W., K. Hubner, C. Schmoor, and M. Schumacher. Validation of existing and development of new prognostic classification schemes in node negative breast cancer. Breast Cancer Res. Treat. 42, 149-163, 1997.
6. Gilchrist, K. W., L. Kalish, V. E. Gould, S. Hirschl, J. E. Imbriglia, W. M. Levy, A. S. Patchefsky, D. W. Penner, J. Pickren, J. A. Roth, and e. al. Interobserver reproducibility of histopathological features in stage II breast cancer. An ECOG study. Breast Cancer Res. Treat. 5, 3-10, 1985.
7. Buettner, P., C. Garbe, and Guggenmoos-Holzmann. Problems in defining cutoff points of continuous prognostic factors: Example of tumour thickness in primary cutaneous melanoma. J Clin. Epidemiology 50, 1201-1210, 1997.
8. Perou, C. M., T. Sorlie, M. B. Eisen, v. d. R. M., S. S. Jeffrey, C. A; Rees, J. R. Pollack, D. T. Ross, H. Johnsen, L. A. Akslen, O. Fluge, A. Pergamenschikov, C. Williams, S. X. Zhu, P. E. Lonning, A. L. Borresen-Dale, P. O. Brown, and D. Botstein. Molecular Portraits of Human Breast Tumours. Nature 406, 747-752, 2000.
9. Gruvberger, S., M. Ringner, Y. Chen, S. Panavally, L. H. Saal, A. Borg, M. Ferno, C. Peterson, and P. Meltzer. Estrogen Receptor Status in Breast Cancer is Associated with Remarkably Distinct Gene Expression Patterns. Cancer Research 61, 5979-5984, 2001.
10. van't Veer, L. J., H. Dai, M. J. van de Vijver, Y. D. He, A. A. M. Hart, M. Mao, H. L. Peterse, K. van der Kooy, M. J. Marton, A. T. Witteveen, G. J. Schreiber, R. M. Kerkhoven, C. Roberts, P. S. Linsley, R. Bernards, and S. H. Friend. Gene expression profiling predicts clinical outcome of breast cancer. Nature 415, 530-536, 2002.
11. West, M., C. Blanchette, H. Dressman, E. Huang, S. Ishida, R. Spang, H. Zuzan, J. A. J. Olson, J. R. Marks, and J. R. Nevins. Predicting the clinical status of human breast cancer by using gene expression profiles. Proc Natl Acad Sci 98, 11462-11467, 2001.
12. Tusher, V. G., R. Tibshirani, and G. Chu. Significance analysis of microarrays applied to the ionizing radiation response. Proc Natl Acad Sci 98, 5116-5121, 2001.
13. Golub, T. R., D. K. Slonim, P. Tamayo, C. Huard, J. P. Gaasenbeek, H. Coller, M. L. Loh, J. R. Downling, M. A. Caligiuri, C. D. Bloomfield, and E. S. Molecular Classification of Cancer: Class Discovery and Class Prediction by Gene Expression Monitoring. Science 286, 531-537, 1999.
14. Sorlie, T., C. M. Perou, R. Tibshirani, T. Aas, S. Geisler, H. Johnsen, T. Hastie, M. B. Eisen, M. van de Rijn, S. S. Jeffrey, T. Thorsen, H. Quist, J. C. Matese, P. O. Brown, D. Botstein, P. E. Lonning, and A. L. Borresen-Dale.

Gene Expression Patterns of Breast Carcinomas Distinguish Tumour Subclasses with Clinical Implications. Proc. Natl. Acad. Sci. 98, 10879-10874, 2001.
15. Yousef, G. M., A. Scorilas, L. G. Kyriakopoulou, L. Rendl, M. Diamandis, R. Ponzone, N. Biglia, M. Giai, R. Roagna, P. Sismondi, and E. P. Diamandis. Human kallikrein gene 5 (KLK5) expression by quantitative PCR: an independent indicator of poor prognosis in breast cancer. Clin Chem 48, 1241-1250, 2002.
16. Martin, K. J., B. M. Kritzman, L. M. Price, B. Koh, C. P. Kwan, X. Zhang, A. Mackay, M. J. O'Hare, C. M. Kaelin, G. L. Mutter, A. B. Pardee, and R. Sager. Linking gene expression patterns to therapeutic groups in breast cancer. Cancer Res., 60, 2232-2238, 2000.
17. Sundquist, M., S. Thorstenson, L. Brudin, and B. Nordenskjold. Applying the Nottingham Prognostic Index to a Swedish breast cancer population. Breast Cancer Res Treat 53, 1-8, 1999.
18. Barbareschi, M., O. Caffo, S. Veronese, R. D. Leek, P. Fina, S. Fox, M. Bonzanini, S. Girlando, L. Morelli, C. Eccher, F. Pezzella, C. Doglioni, P. Dalla Palma, and A. Harris. Bcl-2 and p53 expression in node-negative breast carcinoma: a study with long-term follow-up. Hum. Pathol. 27, 1149-1155, 1996.
19. Frkovic-Grazio, S. and M. Bracko. Long term prognostic value of Nottingham histological grade and its components in early (pT1NOMO) breast carcinoma. J Clin Pathol 55, 88-92, 2002.
20. Ramaswamy, S., K. N. Ross, E. S. Lander, and T. R. Golub. A molecular signature of metastasis in primary solid tumours. Nat Genet 33, 49-54, 2003.
21. Travassoli, F. A. and Schnitt S. J. (1992) Pathology of the Breast In (Elsevier)

22. Eisen M B, Spellman P T, Brown P O, Botstein D. (1998) Cluster analysis and display of genome-wide expression patterns. Proc Natl Acad Sci USA. 95(25), 14863-14868.

TABLE 1a
Association of NPI-ES Expression and NPI status in Rosetta and
Stanford ER+ tumors. The 1^{st} column represents the
number of tumors expressing high or low levels of the NPI-ES.

| | Student's | | | | |
|---|---|---|---|---|---|
| | t-test (continuous) | | | | |
| | mean | | Chi-square (binary) | | |
| Rosetta | (variance) | p = 0.0004 | Low (<3.4) | High | p = 0.0087 |
| High | 3.1 ± 0.4 | | 13 | 11 | |
| (24*) | | | | | |
| Low (25) | 2.3 ± 0.6 | | 22 | 3 | |
| Stanford | | P = 0.001 | | | |
| High (13) | 5.3 ± 0.5 | | | | |
| Low (33) | 4.5 ± 0.6 | | | | |

*Figure in parenthesis represents the no. of samples.

TABLE 1b
Odds ratio for distant metastasis within five years as a first
event in Rosetta ER+ Tumors based upon classical
NPI staging and NPI-ES expression

| | ER+ Tumors | | Odds Ratio* |
|---|---|---|---|
| | Free>5 YR | <5 Yr | (95% CI) |
| NPI (p = 0.06) | | | 6.08 (1.58-23.39) |
| Low (<3.4) | 27 | 8 | |
| High (>=3.4) | 5 | 9 | |
| NPI-ES (p < 0.001) | | | 10.27 (2.40-43.94) |
| Low | 22 | 3 | |
| High | 10 | 14 | |

*Odd ratios were calculated using a standard two-by-two table. CI stands for “confidence interval”.

TABLE 1c
Odds ratio for relapse within five years as a first event in Stanford
ER+ Tumors based upon PES expression and NPI-ES
expression. One sample did not possess relapse information
and was removed from analysis (leaving 45 ER+ tumors).

| | |
|---|---|
| ER+ Tumors | Odds Ratio |

| | Free | Relapse | (95% CI) |
|---|---|---|---|
| PES (p = 0.053) | | | 3.90 (0.94-16.25) |
| Low | 26 | 8 | |
| High | 5 | 6 | |
| NPI-ES (p = 0.040) | | | 4.17 (1.05-16.48) |
| Low | 25 | 7 | |
| High | 6 | 7 | |

TABLE S1
Histopathology of Breast Tumors*

| | | | | | | | | | |
|---|---|---|---|---|---|---|---|---|---|
| Age | Size (mm) | Grade | Node | NPI | ER | PR | Subtype | LVI | DCIS |

ER+

| | | | | | | | | | | |
|---|---|---|---|---|---|---|---|---|---|---|
| 2000220 | 52 | 60 | 3 | 30 of 34 | 7.2 | pos | neg | ductal | yes | minimal |
| 980278 | 64 | 40 | 3 | 14 of 20 | 6.8 | pos | neg | ductal/ | yes | minimal |
| | | | | | | | | micropap | | |
| 2000597 | 57 | 40 | 2 | 0 of 12 | 3.8 | pos | neg | ductal | possible | extensive |
| 2000609 | 62 | 70 | 2 | 17 of 17 | 6.4 | pos | pos | ductal | yes | none |
| 20020071 | 58 | 28 | 3 | 0 of 16 | 4.56 | pos | pos | ductal | no | none |
| 20020160 | 86 | 120 | 3 | 0 of 10 | 6.4 | pos | pos | lobular | no | none |
| 2000787 | 57 | 60 | 3 | 0 of 9 | 5.2 | pos | pos | ductal | yes | none |
| 2000818 | 52 | 10 | 2 | 0 of 11 | 3.2 | pos | neg | ductal | no | minimal |
| 20020051 | 38 | 50 | 3 | 1 of 25 | 6 | pos | pos | ductal | no | none |
| 20020056 | 71 | 20 | 1 | 2 of 17 | 3.4 | pos | neg | ductal | no | minimal |
| 980197 | 55 | 30 | 3 | 2 of 4 | 5.6 | pos | pos | ductal | yes | minimal |
| 980261 | 60 | 15 | 2 | 0 of 9 | 3.3 | pos | neg | ductal | no | minimal |
| 980391 | 56 | 20 | 2 | 0 of 7 | 3.4 | pos | pos | ductal | no | none |
| 2000768 | 39 | 40 | 3 | 0 of 17 | 4.8 | pos | pos | ductal | no | minimal |
| 2000779 | 48 | 55 | 3 | 0 of 14 | 5.1 | pos | neg | ductal | no | none |
| 990123 | 54 | 55 | 3 | 7 of 11 | 7.1 | pos | pos | ductal | no | none |
| 2000422 | 51 | 63 | 3 | 3 of 7 | 6.26 | pos | pos | ductal | no | minimal |
| 2000683 | 72 | 35 | 2 | 0 of 17 | 3.7 | pos | pos | ductal | no | minimal |
| 2000775 | 51 | 25 | 2 | 0 of 12 | 3.5 | pos | neg | ductal | no | minimal |
| 2000804 | 39 | 40 | 3 | 5 of 21 | 6.8 | pos | pos | ductal | yes | minimal |
| 980346 | 52 | 20 | 3 | 0 of 4 | 4.4 | pos | pos | ductal | possible | minimal |
| 980383 | 64 | 30 | 2 | 0 of 16 | 3.6 | pos | pos | ductal | no | minimal |
| 990082 | 49 | 34 | 2 | 3 of 16 | 4.68 | pos | pos | ductal | no | minimal |
| 980177 | 75 | 26 | 2 | 6 of 13 | 5.52 | pos | pos | ductal | yes | none |
| 980178 | 69 | 32 | 3 | 2 of 15 | 5.74 | pos | neg | ductal | no | minimal |
| 980403 | 73 | 30 | 3 | 0 of 9 | 4.6 | pos | pos | ductal | possible | minimal |
| 980434 | 73 | 30 | 3 | 0 of 16 | 4.6 | pos | pos | ductal | no | minimal |
| 990075 | 66 | 25 | 3 | 5 of 21 | 6.5 | pos | pos | ductal | yes | none |
| 990113 | 70 | 90 | 3 | 11 of 15 | 7.8 | pos | pos | ductal | no | minimal |
| 990107 | 50 | 40 | 1 | 1 of 18 | 3.8 | pos | neg | tub-mixed | yes | minimal |
| 980208 | 42 | 25 | 3 | 5 of 20 | 6.5 | pos | pos | ductal | no | none |
| 980220 | 40 | 37 | 2 | 0 of 5 | 3.74 | pos | pos | ductal | yes | minimal |
| 980221 | 33 | 65 | 3 | 1 of 13 | 6.3 | pos | pos | ductal | no | none |
| 990375 | 38 | 15 | 1 | 0 of 10 | 2.3 | pos | neg | ductal | no | extensive |

ER−

| | | | | | | | | | | |
|---|---|---|---|---|---|---|---|---|---|---|
| 980193 | 49 | 25 | 3 | 3 of 23 | 5.5 | neg | neg | ductal | no | minimal |
| 980216 | 65 | 45 | 2 | 5 of 20 | 5.9 | neg | neg | ductal | no | none |
| 980256 | 46 | 36 | 3 | 1 of 12 | 5.72 | neg | neg | ductal | no | none |
| 980285 | 49 | 40 | 3 | 1 of 7 | 5.8 | neg | neg | ductal | yes | minimal |
| 980338 | 55 | 30 | 3 | 0 of 7 | 4.6 | neg | neg | ductal | no | none |
| 980353 | 58 | 45 | 3 | 0 of 25 | 4.9 | neg | neg | metaplastic | no | none |
| 980411 | 69 | 30 | 2 | 0 of 9 | 3.6 | neg | neg | ductal | no | none |
| 980441 | 66 | 30 | 3 | 4 of 14 | 6.6 | neg | neg | ductal | yes | none |
| 990174 | 55 | 45 | 2 | 3 of 24 | 5.9 | neg | neg | ductal | yes | minimal |
| 2000320 | 67 | 20 | 3 | 20 of 21 | 6.4 | neg | neg | ductal | yes | none |
| 2000500 | 44 | 75 | 3 | 6 of 6 | 7.5 | neg | neg | ductal | yes | none |
| 980247 | 35 | 45 | 3 | 1 of 19 | 5.9 | neg | neg | ductal | yes | minimal |
| 990299 | 58 | 55 | 3 | 7 of 17 | 7.1 | neg | neg | ductal | possible | minimal |
| 2000593 | 60 | 41 | 3 | 0 of 15 | 4.82 | neg | neg | ductal | no | none |
| 2000638 | 60 | 40 | 1 | 0 of 15 | 2.8 | pos | neg | lobular | no | none |
| 2000731 | 68 | 51 | 3 | 1 of 29 | 6.02 | pos | neg | ductal | no | minimal |
| 2000880 | 55 | 15 | 2 | 0 of 26 | 3.3 | neg | neg | ductal | no | none |

ERBB2

| | | | | | | | | | | |
|---|---|---|---|---|---|---|---|---|---|---|
| 980194 | 58 | 50 | 3 | 25 of 32 | 7 | neg | neg | ductal | yes | none |
| 980214 | 49 | 60 | 2 | 5 of 13 | 6.2 | pos | neg | ductal | no | extensive |
| 980238 | 62 | 20 | 3 | 7 of 21 | 6.4 | neg | neg | ductal | no | extensive |
| 980288 | 45 | 60 | 3 | 13 of 15 | 7.2 | pos | neg | ductal | yes | extensive |
| 980335 | 33 | 3 | 3 | 3 of 7 | 5.06 | neg | neg | ductal | yes | extensive |
| 980373 | 77 | 30 | 3 | 0 of 14 | 4.6 | neg | neg | ductal | no | minimal |
| 980380 | 56 | | | 0 of 6 | | neg | neg | | | |
| 980395 | 68 | 30 | 3 | 1 of 10 | 5.6 | neg | neg | ductal | yes | none |
| 980396 | 66 | 35 | 3 | 10 of 12 | 6.7 | neg | neg | ductal | yes | extensive |
| 990115 | 38 | 28 | 3 | 9 of 10 | 6.56 | pos | pos | ductal | yes | extensive |
| 990134 | 43 | 40 | 3 | 0 of 19 | 4.8 | neg | neg | ductal | no | none |
| 990148 | 60 | 40 | 2 | 6 of 19 | 5.8 | pos | neg | ductal | yes | minimal |
| 990223 | 52 | 5 | 3 | 1 of 21 | 5.1 | pos | neg | ductal | no | extensive |
| 2000104 | 59 | | | | | pos | neg | ductal | | |
| 2000171 | 50 | 25 | 2 | 0 of 9 | 3.5 | neg | neg | ductal | no | none |
| 2000209 | 58 | 50 | 3 | 0 of 7 | 5 | pos | neg | ductal | no | none |
| 2000210 | 50 | 40 | 3 | 3 of 6 | 5.8 | neg | neg | ductal | yes | none |
| 2000237 | 43 | 47 | 3 | 23 of 40 | 6.94 | pos | pos | ductal | yes | minimal |
| 2000287 | 53 | 40 | 3 | 0 of 8 | 4.8 | neg | neg | ductal | possible | none |
| 2000399 | 44 | 40 | 2 | 0 of 8 | 3.8 | neg | neg | ductal | no | minimal |
| 2000641 | 47 | 60 | 3 | 16 of 24 | 5.2 | neg | neg | ductal | yes | minimal |
| 2000652 | 56 | 25 | 3 | 6 of 21 | 6.5 | neg | neg | ductal | no | minimal |
| 2000675 | 78 | 55 | 3 | 16 of 16 | 7.1 | neg | neg | ductal | yes | minimal |
| 2000709 | 45 | 30 | 3 | 0 of 16 | 4.6 | neg | neg | ductal | no | none |
| 2000759 | 57 | 7 | 3 | 0 of 12 | 4.14 | neg | neg | ductal | no | extensive |
| 2000813 | 60 | 23 | 3 | 16 of 17 | 6.46 | neg | neg | ductal | yes | extensive |
| 2000829 | 51 | 45 | 2 | 10 of 10 | 5.9 | neg | neg | ductal | yes | extensive |
| 20020090 | 60 | 45 | 3 | 19 of 27 | 6.9 | neg | neg | ductal | yes | minimal |

*This list contains clinical information for 79 out of 98 tumors used in this study. Clinical information for the remaining 19 tumors was incomplete and not included in this list. Only the 79 samples with complete clinical information was used for subsequent NPI-ES analysis.

Table S3, the NPI-ES delivered a classification accuracy of 80%, compared to the 44 gene set which delivered a 70% classification accuracy.

TABLE S3
Classification accuracy of the NPI-ES or 44 gene set
on 78 Rosetta Tumors

| | NPI classification (<3.4 or >3.4) |
|---|---|
| | No. of misclassifications (Accuracy) |
| 44 Genes | 23 (70%) |
| NPI-ES | 15 (80%) |

TABLE S5
List of top 50 Significantly Regulated Genes in ER+, ER−
and ERBB2+ Molecular Subtypes
This list represents the top 50 genes identified by SAM to be significantly regulated in
each molecular subtype (ER+, ER−, ERBB2+). The genes are ranked by their S2N
correlation ratio, which reflects the extent of the expression perturbation observed among
different groups. There is good overlap between these genes and similar lists reported by
other studies (ref. 8-11) (main text).

| Gene description | Unigene | Chromosome |
|---|---|---|
| ER+ Molecular Subtype | | |
| estrogen receptor 1 | Hs.1657 | Chr: 6q25.1 |
| GATA binding protein 3 | Hs.169946 | Chr: 10p15 |
| annexin A9 | Hs.279928 | Chr: 1q21 |
| KIAA0882 protein | Hs.90419 | Chr: 4q31.1 |
| carbonic anhydrase XII | Hs.5338 | Chr: 15q22 |
| cytochrome P450, subfamily IIB (phenobarbital-inducible), polypeptide 6 | Hs.1360 | Chr: 19q13.2 |
| dynein, axonemal, light intermediate polypeptide 1 | Hs.406050 | Chr: 1p35.1 |
| sema domain, immunoglobulin domain (Ig), short basic domain, | Hs.82222 | Chr: 3p21.3 |
| secreted, (semaphorin) 3B | | |
| N-acetyltransferase 1 (arylamine N-acetyltransferase) | Hs.155956 | Chr: 8p23.1-p21.3 |
| serine (or cysteine) proteinase inhibitor, clade A (alpha-1 | Hs.76353 | Chr: 14q32.1 |
| antiproteinase, antitrypsin), member 5 | | |
| cytochrome c oxidase subunit VIc | Hs.351875 | Chr: 8q22-q23 |
| Homo sapiens mRNA; cDNA DKFZp564F053 (from clone | Hs.71968 | — |
| DKFZp564F053), mRNA sequence | | |
| LIV-1 protein, estrogen regulated | Hs.79136 | Chr: 18q12.1 |
| troponin T1, skeletal, slow | Hs.73980 | Chr: 19q13.4 |
| hypothetical protein FLJ20151 | Hs.279916 | Chr: 15q21.3 |
| calsyntenin 2 | Hs.12079 | Chr: 3q23-q24 |
| B-cell CLL/lymphoma 2 | Hs.79241 | Chr: 18q21.3 |
| guanidinoacetate N-methyltransferase | Hs.81131 | Chr: 19p13.3 |
| microtubule-associated protein tau | Hs.101174 | Chr: 17q21.1 |
| hypothetical protein FLJ12910 | Hs.15929 | Chr: 6q25.1 |
| WW domain-containing protein 1 | Hs.355977 | Chr: 8q21 |
| UDP-glucose ceramide glucosyltransferase | Hs.432605 | Chr: 9q31 |
| GREB1 protein | Hs.193914 | Chr: 2p25.1 |
| RNB6 | Hs.241471 | Chr: 14q32.32 |
| Human insulin-like growth factor 1 receptor mRNA, 3′ sequence, | Hs.405998 | — |
| mRNA sequence | | |
| interleukin 6 signal transducer (gp130, oncostatin M receptor) | Hs.82065 | Chr: 5q11 |
| LAG1 longevity assurance homolog 2 (S. cerevisiae) | Hs.285976 | Chr: 1q21.2 |
| cadherin, EGF LAG seven-pass G-type receptor 2 (flamingo homolog, | Hs.57652 | Chr: 1p21 |
| Drosophila) | | |
| paired basic amino acid cleaving system 4 | Hs.170414 | Chr: 15q26 |
| regulator of G-protein signalling 11 | Hs.65756 | Chr: 16p13.3 |
| UDP-glucose ceramide glucosyltransferase | Hs.432605 | Chr: 9q31 |
| NPD009 protein | Hs.283675 | Chr: 16p13.2 |
| v-myb myeloblastosis viral oncogene homolog (avian) | Hs.1334 | Chr: 6q22-q23 |
| interleukin 6 signal transducer (gp130, oncostatin M receptor) | Hs.82065 | Chr: 5q11 |
| discs, large (Drosophila) homolog 5 | Hs.170290 | Chr: 10q23 |
| Homo sapiens mRNA; cDNA DKFZp434E082 (from clone | Hs.432587 | — |
| DKFZp434E082), mRNA sequence | | |
| cytochrome P450, subfamily IIB (phenobarbital-inducible), polypeptide 7 | Hs.330780 | Chr: 19q13.2 |
| HSPC009 protein | Hs.16059 | Chr: 17q21 |
| KIAA1025 protein | Hs.4084 | Chr: 12q24.22 |
| protein tyrosine phosphatase type IVA, member 2 | Hs.82911 | Chr: 1p35 |
| CGI-49 protein | Hs.238126 | Chr: 1q44 |
| chromosome 20 open reading frame 35 | Hs.256086 | Chr: 20q13.11 |
| phorbol-12-myristate-13-acetate-induced protein 1 | Hs.96 | Chr: 18q21.31 |
| KIAA0876 protein | Hs.301011 | Chr: 19p13.3 |
| hypothetical protein FLJ20152 | Hs.82273 | Chr: 5p15.1 |
| hypothetical protein FLJ22315 | Hs.22753 | Chr: 5q35.3 |
| trefoil factor 1 (breast cancer, estrogen-inducible sequence expressed | Hs.350470 | Chr: 21q22.3 |
| in) | | |
| polymerase (DNA-directed), delta 4 | Hs.82520 | Chr: 11q13 |
| putative proline 4-hydroxylase | Hs.348198 | Chr: 3p21.31 |
| GDNF family receptor alpha 1 | Hs.105445 | Chr: 10q26 |
| ERBB2+ Molecular Subtype | | |
| chloride channel, calcium activated, family member 2 | Hs.241551 | Chr: 1p31-p22 |
| v-erb-b2 erythroblastic leukemia viral oncogene homolog 2, | Hs.323910 | Chr: 17q11.2-q12 |
| neuro/glioblastoma derived oncogene homolog (avian) | | |
| growth factor receptor-bound protein 7 | Hs.86859 | Chr: 17q21.1 |
| dual specificity phosphatase 6 | Hs.180383 | Chr: 12q22-q23 |
| START domain containing 3 | Hs.77628 | Chr: 17q11-q12 |
| transient receptor potential cation channel, subfamily V, member 6 | Hs.302740 | Chr: 7q33-q34 |
| S100 calcium binding protein A8 (calgranulin A) | Hs.100000 | Chr: 1q21 |
| protein phosphatase 1, regulatory (inhibitor) subunit 1A | Hs.76780 | Chr: 12q13.13 |
| fibroblast growth factor receptor 4 | Hs.165950 | Chr: 5q35.1-qter |
| SRY (sex determining region Y)-box 11 | Hs.32964 | Chr: 2p25 |
| Unknown protein [Homo sapiens], mRNA sequence | Hs.106642 | — |
| transducin-like enhancer of split 1 (E(sp1) homolog, Drosophila) | Hs.28935 | Chr: 9q21.32 |
| hypothetical gene MGC9753 | Hs.91668 | Chr: 17q21.1 |
| mitogen-activated protein kinase kinase kinase 5 | Hs.151988 | Chr: 6q22.33 |
| KIAA1102 protein | Hs.202949 | Chr: 4p13 |
| fatty acid hydroxylase | Hs.249163 | Chr: 16q23 |
| transcription factor AP-2 beta (activating enhancer binding protein 2 | Hs.33102 | Chr: 6p12 |
| beta) | | |
| S100 calcium binding protein A9 (calgranulin B) | Hs.112405 | Chr: 1q21 |
| fatty-acid-Coenzyme A ligase, long-chain 2 | Hs.154890 | Chr: 4q34-q35 |
| hypothetical protein FLJ22671 | Hs.193745 | Chr: 2q37.3 |
| kynurenine 3-monooxygenase (kynurenine 3-hydroxylase) | Hs.107318 | Chr: 1q42-q44 |
| KIAA0644 gene product | Hs.21572 | Chr: 7p15.1 |
| aspartate beta-hydroxylase | Hs.283664 | Chr: 8q12.1 |
| electron-transfer-flavoprotein, alpha polypeptide (glutaric aciduria II) | Hs.169919 | Chr: 15q23-q25 |
| secretory leukocyte protease inhibitor (antileukoproteinase) | Hs.251754 | Chr: 20q12 |
| isocitrate dehydrogenase 1 (NADP+), soluble | Hs.11223 | Chr: 2q33.3 |
| phenylethanolamine N-methyltransferase | Hs.1892 | Chr: 17q21-q22 |
| hypothetical protein FLJ14146 | Hs.103395 | Chr: 1q42.11 |
| fucosyltransferase 3 (galactoside 3(4)-L-fucosyltransferase, Lewis | Hs.169238 | Chr: 19p13.3 |
| blood group included) | | |
| keratin, hair, basic, 1 | Hs.32952 | Chr: 12q13 |
| PDZ domain containing 2 | Hs.173035 | Chr: 5p13.3 |
| argininosuccinate synthetase | Hs.160786 | Chr: 9q34.1 |
| specific granule protein (28 kDa) | Hs.54431 | Chr: 6p12.3 |
| Homo sapiens cDNA: FLJ21521 fis, clone COL05880, mRNA | Hs.306777 | — |
| sequence | | |
| kynureninase (L-kynurenine hydrolase) | Hs.169139 | Chr: 2q22.1 |
| hypothetical protein FLJ20539 | Hs.118552 | Chr: 11q12.1 |
| proline dehydrogenase (oxidase) 1 | Hs.343874 | Chr: 22q11.21 |
| v-myc myelocytomatosis viral related oncogene, neuroblastoma | Hs.25960 | Chr: 2p24.1 |
| derived (avian) | | |
| integrin, beta 6 | Hs.57664 | Chr: 2q24.2 |
| hypothetical protein MGC3077 | Hs.433404 | Chr: 7p15-p14 |
| uncoupling protein 2 (mitochondrial, proton carrier) | Hs.80658 | Chr: 11q13 |
| myosin X | Hs.61638 | Chr: 5p15.1-p14.3 |
| keratin 7 | Hs.23881 | Chr: 12q12-q21 |
| steroid sulfatase (microsomal), arylsulfatase C, isozyme S | Hs.79876 | Chr: Xp22.32 |
| formin homology 2 domain containing 1 | Hs.95231 | Chr: 16q22 |
| ATP-binding.cassette, sub-family C (CFTR/MRP), member 3 | Hs.90786 | Chr: 17q22 |
| chondroitin beta1,4 N-acetylgalactosaminyltransferase | Hs.11260 | Chr: 8p21.3 |
| KIAA0485 protein | Hs.89121 | — |
| kraken-like | Hs.301947 | Chr: 22q13 |
| collagen, type XIII, alpha 1 | Hs.211933 | Chr: 10q22 |
| ER− Molecular Subtype | | |
| keratin 16 (focal non-epidermolytic palmoplantar keratoderma) | Hs.432448 | Chr: 17q12-q21 |
| gamma-aminobutyric acid (GABA) A receptor, pi | Hs.70725 | Chr: 5q33-q34 |
| TONDU | Hs.9030 | Chr: Xq26.3 |
| keratin 6B | Hs.432677 | Chr: 12q12-q13 |
| serine (or cysteine) proteinase inhibitor, clade B (ovalbumin), member 5 | Hs.55279 | Chr: 18q21.3 |
| keratin 5 (epidermolysis bullosa simplex, Dowling- | Hs.433845 | Chr: 12q12-q13 |
| Meara/Kobner/Weber-Cockayne types) | | |
| SRY (sex determining region Y)-box 10 | Hs.44317 | Chr: 22q13.1 |
| melanoma inhibitory activity | Hs.279651 | Chr: 19q13.32-q13.33 |
| matrix metalloproteinase 7 (matrilysin, uterine) | Hs.2256 | Chr: 11q21-q22 |
| secreted frizzled-related protein 1 | Hs.7306 | Chr: 8p12-p11.1 |
| B-cell CLL/lymphoma 11A (zinc finger protein) | Hs.130881 | Chr: 2p15 |
| Homo sapiens cDNA FLJ11796 fis, clone HEMBA1006158, highly | Hs.284186 | — |
| similar to Homo sapiens transcription factor forkhead-like 7 (FKHL7) | | |
| gene, mRNA sequence | | |
| solute carrier family 6 (neurotransmitter transporter), member 14 | Hs.162211 | Chr: Xq23-q24 |
| desmuslin | Hs.10587 | Chr: 15q26.3 |
| engrailed homolog 1 | Hs.271977 | Chr: 2q13-q21 |
| ribosomal protein, large P2 | Hs.153179 | Chr: 11p15.5-p15.4 |
| tripartite motif-containing 29 | Hs.82237 | Chr: 11q22-q23 |
| calmodulin-like skin protein | Hs.180142 | Chr: 10p15.1 |
| desmocollin 2 | Hs.239727 | Chr: 18q12.1 |
| ropporin, rhophilin associated protein | Hs.194093 | Chr: 3q21.1 |
| crystallin, alpha B | Hs.391270 | Chr: 11q22.3-q23.1 |
| tripartite motif-containing 2 | Hs.12372 | Chr: 4q31.23 |
| epidermal growth factor receptor (erythroblastic leukemia viral (v-erb- | Hs.77432 | Chr: 7p12 |
| b) oncogene homolog, avian) | | |
| leucine-rich acidic nuclear protein like | Hs.71331 | Chr: 1q21.2 |
| potassium channel, subfamily K, member 5 | Hs.127007 | Chr: 6p21 |
| kallikrein 5 | Hs.50915 | Chr: 19q13.3-q13.4 |
| procollagen C-endopeptidase enhancer 2 | Hs.8944 | Chr: 3q21-q24 |
| Hypothetical protein [Homo sapiens], mRNA sequence | Hs.66762 | — |
| LIM domain only 4 | Hs.3844 | Chr: 1p22.3 |
| keratin 17 | Hs.2785 | Chr: 17q12-q21 |
| desmoglein 3 (pemphigus vulgaris antigen) | Hs.1925 | Chr: 18q12.1-q12.2 |
| keratin 6A | Hs.367762 | Chr: 12q12-q13 |
| sialyltransferase 8A (alpha-N-acetylneuraminate: alpha-2,8- | Hs.82527 | Chr: 12p12.1-p11.2 |
| sialytransferase, GD3 synthase) | | |
| Kruppel-like factor 5 (intestinal) | Hs.84728 | Chr: 13q21.32 |
| Rho guanine nucleotide exchange factor (GEF) 4 | Hs.6066 | Chr: 2q22 |
| kallikrein 6 (neurosin, zyme) | Hs.79361 | Chr: 19q13.3 |
| prostaglandin-endoperoxide synthase 2 (prostaglandin G/H synthase | Hs.196384 | Chr: 1q25.2-q25.3 |
| and cyclooxygenase) | | |
| chromosome 20 open reading frame 42 | Hs.180479 | Chr: 20p12.3 |
| glycoprotein M6B | Hs.5422 | Chr: Xp22.2 |
| uridine phosphorylase | Hs.77573 | Chr: 7 |
| ladinin 1 | Hs.18141 | Chr: 1q25.1-q32.3 |
| pleiomorphic adenoma gene-like 1 | Hs.75825 | Chr: 6q24-q25 |
| desmocollin 3 | Hs.41690 | Chr: 18q12.1 |
| Homo sapiens cDNA FLJ30869 fis, clone FEBRA2004224, mRNA | Hs.349611 | — |
| sequence | | |
| HRAS-like suppressor | Hs.36761 | Chr: 3q29 |
| cysteine and glycine-rich protein 2 | Hs.10526 | Chr: 12q21.1 |
| scrapie responsive protein 1 | Hs.7122 | Chr: 4q31-q32 |
| amyloid beta (A4) precursor protein-binding, family A, member 2 (X11- | Hs.26468 | Chr: 15q11-q12 |
| like) | | |
| jerky homolog-like (mouse) | Hs.105940 | Chr: 11q21 |
| transforming growth factor, alpha | Hs.170009 | Chr: 2p13 |

TABLE S6
Genes Belonging to the NPI-ES (62 Genes)
DC13 protein is the only gene of NPI-ES that can be matched in Rosetta 70-gene
‘prognosis’ signature (PES, see main text), out of which 42 are present in the Affymetrix
U133A chip.

| Gene Description | Unigene | Biological Process (GO) |
|---|---|---|
| Positive genes (60) (Highly Expressed in High NPI Tumors) | | |
| adenine phosphoribosyltransferase | Hs.28914 | 9116 // nucleoside metabolism // |
| | | extended: inferred from electronic annotation; |
| | | Pribosyltran; 5e−44 |
| MCM4 minichromosome maintenance deficient 4 (S. cerevisiae) | Hs.154443 | 6260 // DNA replication // |
| | | predicted/computed |
| exonuclease 1 | Hs.47504 | 6310 // DNA recombination // experimental |
| | | evidence /// 6281 // DNA repair // |
| | | experimental evidence /// 6298 // mismatch |
| | | repair // predicted/computed |
| Metallothionein 1H-like protein [Homo sapiens], mRNA | Hs.367850 | — |
| sequence | | |
| Homo sapiens, clone IMAGE: 5270727, mRNA, mRNA | Hs.319215 | — |
| sequence | | |
| DC13 protein | Hs.6879 | — |
| HSPC037 protein | Hs.433180 | — |
| H2A histone family, member Z | Hs.119192 | — |
| discs, large homolog 7 (Drosophila) | Hs.77695 | 7267 // cell-cell signaling // |
| | | extended: Unknown; GKAP; 2.1e−05 |
| RNA helicase-related protein [Homo sapiens], mRNA sequence | Hs.381097 | — |
| kinesin-like 1 | Hs.8878 | 7067 // mitosis // experimental evidence /// |
| | | 7052 // mitotic spindle assembly // |
| | | experimental evidence |
| chromosome 20 open reading frame 1 | Hs.9329 | 7067 // mitosis // predicted/computed /// 8283 |
| | | // cell proliferation // predicted/computed |
| KIAA0095 gene product | Hs.155314 | — |
| helicase, lymphoid-specific | Hs.203963 | — |
| homeo box HB9 | Hs.37035 | 6959 // humoral immune response // |
| | | experimental evidence /// 6357 // regulation |
| | | of transcription from Pol II promoter // |
| | | predicted/computed /// 7345 // |
| | | embryogenesis and morphogenesis // |
| | | experimental evidence |
| DNA segment on chromosome X (unique) 9879 expressed | Hs.18212 | — |
| sequence | | |
| MAD2 mitotic arrest deficient-like 1 (yeast) | Hs.79078 | 7067 // mitosis // predicted/computed /// 7093 |
| | | // mitotic checkpoint // experimental evidence |
| eukaryotic translation initiation factor 4E binding protein 1 | Hs.433317 | 6445 // regulation of translation // |
| | | predicted/computed |
| cathepsin C | Hs.10029 | 6508 // proteolysis and peptidolysis // not |
| | | recorded /// 6955 // immune response // |
| | | experimental evidence |
| H2B histone family, member J | Hs.249216 | — |
| proteasome (prosome, macropain) subunit, beta type, 8 (large | Hs.180062 | 6508 // proteolysis and peptidolysis // not |
| multifunctional protease 7) | | recorded |
| hypothetical protein FLJ20105 | Hs.89306 | — |
| chromosome 10 open reading frame 3 | Hs.14559 | — |
| uncharacterized bone marrow protein BM039 | Hs.283532 | — |
| likely ortholog of mouse gene rich cluster, C8 gene | Hs.30114 | — |
| cell division cycle 2, G1 to S and G2 to M | Hs.334562 | 74 // regulation of cell cycle // not recorded /// |
| | | 7089 // start control point of mitotic cell cycle |
| | | // not recorded |
| metallothionein 2A | Hs.118786 | 6878 // copper homeostasis // |
| | | predicted/computed |
| geminin, DNA replication inhibitor | Hs.234896 | 7050 // cell cycle arrest // |
| | | predicted/computed /// 8156 // negative |
| | | regulation of DNA replication // |
| | | predicted/computed |
| low density lipoprotein receptor-related protein 8, apolipoproteine | Hs.54481 | 7165 // signal transduction // |
| receptor | | predicted/computed /// 6629 // tipid |
| | | metabolism // predicted/computed |
| hematological and neurological expressed 1 | Hs.109706 | — |
| H1 histone family, member 2 | Hs.7644 | — |
| nudix (nucleoside diphosphate linked moiety X)-type motif 1 | Hs.388 | 6979 // response to oxidative stress // |
| | | predicted/computed /// 6281 // DNA repair // |
| | | not recorded |
| metallothionein 1X | Hs.374950 | — |
| H2B histone family, member T | Hs.247817 | — |
| tetraspan 1 | Hs.38972 | 8283 // cell proliferation // not recorded /// |
| | | 8583 // mystery cell fate differentiation |
| | | (sensu Drosophila) // predicted/computed /// |
| | | 7155 // cell adhesion // not recorded /// 6928 |
| | | // cell motility // not recorded |
| metallothionein 1H | Hs.2667 | — |
| H3 histone family, member K | Hs.70937 | — |
| ribonucleotide reductase M2 polypeptide | Hs.75319 | — |
| baculoviral IAP repeat-containing 5 (survivin) | Hs.1578 | 86 // G2/M transition of mitotic cell cycle // |
| | | experimental evidence /// 7048 // |
| | | oncogenesis // predicted/computed /// 6916 // |
| | | anti-apoptosis // experimental evidence |
| F-box only protein 5 | Hs.272027 | 6508 // proteolysis and peptidolysis // |
| | | predicted/computed |
| serine (or cysteine) proteinase inhibitor, clade A (alpha-1 | Hs.297681 | — |
| antiproteinase, antitrypsin), member 1 | | |
| lysosomal associated protein transmembrane 4 beta | Hs.296398 | — |
| chemokine (C—X3—C motif) ligand 1 | Hs.80420 | 7165 // signal transduction // experimental |
| | | evidence /// 6954 // inflammatory response // |
| | | not recorded /// 6935 // chemotaxis // |
| | | experimental evidence /// 6955 // immune |
| | | response // not recorded /// 7155 // cell |
| | | adhesion // experimental evidence /// 7267 // |
| | | cell-cell signaling // experimental evidence |
| CD27-binding (Siva) protein | Hs.112058 | 8624 // induction of apoptosis by extracellular |
| | | signals // predicted/computed /// 6952 // |
| | | defense response // predicted/computed |
| LGN protein | Hs.278338 | 7186 // G-protein coupled receptor protein |
| | | signaling pathway // predicted/computed |
| Mouse Mammary Turmor Virus Receptor homolog 1 | Hs.18686 | — |
| forkhead box M1 | Hs.239 | 6366 // transcription from Pol II promoter // |
| | | experimental evidence /// 6979 // response to |
| | | oxidative stress // experimental evidence |
| met proto-oncogene (hepatocyte growth factor receptor) | Hs.316752 | 7048 // oncogenesis // experimental evidence |
| | | /// 8283 // cell proliferation // |
| | | predicted/computed /// 7165 // signal |
| | | transduction // predicted/computed |
| butyrophilin, subfamily 3, member A2 | Hs.87497 | — |
| SBBI26 protein | Hs26481 | — |
| likely ortholog of mouse Shc SH2-domain binding protein 1 | Hs.123253 | — |
| H3 histone family, member B | Hs.143042 | — |
| trefoil factor 3 (intestinal) | Ms.82961 | 6952 // defense response // |
| | | predicted/computed /// 7586 // digestion // |
| | | predicted/computed |
| Immunoglobutin lambda locus | Hs.405944 | — |
| DNA replication factor | Hs.122908 | — |
| Homo sapiens cDNA FLJ30781 fis, clone FEBRA2000874, | Hs.301663 | — |
| mRNA sequence | | |
| chemokine (C—C motif) ligand 18 (pulmonary and activation- | Hs.16530 | 7165 // signal transduction // experimental |
| regulated) | | evidence /// 7154 // cell communication // |
| | | predicted/computed /// 6935 // chemotaxis // |
| | | experimental evidence /// 6955 // immune |
| | | response // predicted/computed /// 6960 // |
| | | antimicrobial humoral response (sensu |
| | | Invertebrata) // predicted/computed /// 9607 // |
| | | response to biotic stimulus // |
| | | predicted/computed /// 7267 // cell-cell |
| | | signaling // experimental evidence |
| immunoglobulin kappa constant | Hs.406565 | — |
| suppressor of Ty 4 homolog 1 (S. cerevisiae) | Hs.79058 | 6355 // regulation of transcription, DNA- |
| | | dependent // predicted/computed /// 6357 // |
| | | regulation of transcription from Pol II |
| | | promoter // predicted/computed /// 6338 // |
| | | chromatin modeling // predicted/computed |
| paternally expressed 10 | Hs.137476 | — |
| Negative genes (2) (Highly Expressed in Low NPI Tumors) | | |
| BTG family, member 2 | Hs.75462 | 8285 // negative regulation of cell |
| | | proliferation // predicted/computed /// 6281 // |
| | | DNA repair // predicted/computed /// 6976 // |
| | | DNA damage response, activation of p53 // |
| | | predicted/computed |
| cytochrome P450, subfamily IVF, polypeptide 8 | Hs.268554 | 6118 // electron transport // |
| | | extended: Unknown; p450; 1.9e−142 /// 6693 |
| | | // prostaglandin metabolism // |
| | | predicted/computed |

TABLE S7
SAM was performed to identify 68 genes significantly associated with grade
(FDR of 14%, >=2-fold change). 45 out of these genes (66%) are also belong
to the NPI classifier, labeled as “YES” in the NPI-ES column.

| Gene Name | NPI-ES |
|---|---|
| Genes upregulated in Grade 3 tumors | |
| RAD51-interacting protein | |
| DC13 protein | YES |
| HSPC037 protein | YES |
| homeo box HB9 | YES |
| cyclin B2 | |
| protein regulator of cytokinesis 1 | |
| likely ortholog of mouse gene rich cluster, C8 gene | YES |
| kinesin-like 1 | YES |
| H2A histone family, member Z | YES |
| DNA replication factor | YES |
| MCM4 minichromosome maintenance deficient 4 (S. cerevisiae) | YES |
| discs, large homolog 7 (Drosophila) | YES |
| ZW10 interactor | |
| MAD2 mitotic arrest deficient-like 1 (yeast) | YES |
| Metallothionein 1H-like protein [Homo sapiens], mRNA sequence | YES |
| chromosome 10 open reading frame 3 | YES |
| ribonucleotide reductase M2 polypeptide | YES |
| cell division cycle 2, G1 to S and G2 to M | YES |
| forkhead box M1 | YES |
| uncharacterized bone marrow protein BM039 | YES |
| helicase, lymphoid-specific | YES |
| RNA helicase-related protein [Homo sapiens], mRNA sequence | YES |
| metallothionein 1X | YES |
| Homo sapiens, clone IMAGE: 5270727, mRNA, mRNA sequence | YES |
| metallothionein 2A | YES |
| metallothionein 1H | YES |
| KIAA0095 gene product | YES |
| baculoviral IAP repeat-containing 5 (survivin) | YES |
| geminin, DNA replication Inhibitor | YES |
| enhancer of zeste homolog 2 (Drosophila) | |
| cathepsin C | YES |
| nudix (nucleoside diphosphate linked moiety X)-type motif 1 | YES |
| hypothetical protein FLJ10719 | |
| chemokine (C—X3—C motif) ligand 1 | YES |
| tetraspan 1 | YES |
| proapoptotic caspase adaptor protein | |
| immunoglobulin lambda locus | YES |
| H2B histone family, member J | YES |
| trefoil factor 3 (intestinal) | YES |
| CD27-binding (Siva) protein | YES |
| topoisomerase (DNA) II alpha 170 kDa | |
| Immunoglobulin lambda joining 3 | |
| eukaryotic translation initiation factor 4E binding protein 1 | YES |
| H3 histone family, member K | YES |
| chemokine (C—C motif) ligand 18 (pulmonary and activation-regulated) | YES |
| lysosomal associated protein transmembrane 4 beta | YES |
| Mouse Mammary Turmor Virus Receptor homolog 1 | YES |
| LGN protein | YES |
| Immunoglobulin kappa constant | YES |
| carboxypeptidase B1 (tissue) | |
| met proto-oncogene (hepatocyte growth factor receptor) | YES |
| H2B histone family, member T | YES |
| RAB38, member RAS oncogene family | |
| H1 histone family, member 2 | YES |
| hypothetical protein from EUROIMAGE 2021883 | |
| apolipoprotein B mRNA editing enzyme, catalytic polypeptide-like 3B | |
| H3 histone family, member B | YES |
| Immunoglobulin heavy constant gamma 3 (G3m marker) | |
| similar to bK246H3.1 (immunoglobulin lambda-like polypeptide 1, pre-B-cell | |
| specific) | |
| Immunoglobulin lambda light chain [Homo sapiens], mRNA sequence | |
| Immunoglobulin kappa light chain variable region [Homo sapiens], mRNA | |
| sequence | |
| serine (or cysteine) proteinase inhibitor, clade A (alpha-1 antiproteinase, | YES |
| antitrypsin), member 1 | |
| proteolipid protein 1 (Pelizaeus-Merzbacher disease, spastic paraplegia 2, | |
| uncomplicated) | |
| sodium channel, nonvoltage-gated 1, beta (Liddle syndrome) | |
| H4 histone family, member H | |
| syndecan 2 (heparan sulfate proteoglycan 1, cell surface-associated, | |
| fibroglycan) | |
| neuropilin (NRP) and tolloid (TLL)-like 2 | |
| Genes downregulated in Grade 3 tumors | |
| hypothetical protein FLJ22418 | |

TABLE S11
Correlation of Luminal A and Luminal C Tumors with High
and Low NPI-ES Expression (Luminal Tumors were identified
based upon results of Sorlie et al., (2001))

| | Luminal A | Luminal C |
|---|---|---|
| Low NPI-ES | 30 | 0 |
| High NPI-ES | 2 | 10 |

TABLE S12
The number of deaths (after 5 years) was then tabulated
for each group as follows:

| | H->H | H->L | L->L | L->H |
|---|---|---|---|---|
| Total | 6 | 4 | 10 | N/A |
| Death | 4 | 0 | 3 | N/A |
| AWD* | 1 | 0 | 2 | N/A |

*AWD: alive with disease

TABLE S13
Genes that overlap between prognostic set and Rosetta 231 genes

| accession # | correlation | gene name | description |
|---|---|---|---|
| NM_020188 | −0.40007 | DC13 | DC13 protein |
| NM_001168 | −0.33813 | BIRC5 | baculoviral IAP repeat- |
| | | | containing 5 (survivin) |
| NM_006763 | 0.345013 | BTG2 | BTG family, member 2 |
| NM_012177 | −0.32571 | FBXO5 | F-box only protein 5 |
| NM_013296 | −0.30129 | HSU54999 | LGN protein |
| Contig41413_RC | −0.30837 | RRM2 | ribonucleotide reductase |
| | | | M2 polypeptide |
| NM_018455 | −0.33103 | BM039 | uncharacterized bone |
| | | | marrow protein BM039 |
| NM_002358 | −0.30251 | MAD2L1 | MAD2 (mitotic arrest |
| | | | deficient, yeast, |
| | | | homolog)-like 1 |

FIG. S14: Expression data for the prognostic set (or NPI-ES) of genes across samples of differing
NPI value.

| UID | NAME | 2000220 | 980278 | 2000597 | 2000609 | 20020071 | 20020160 | 2000787 | 2000818 |
|---|---|---|---|---|---|---|---|---|---|
| NPI | | 7.2 | 6.8 | 3.8 | 6.4 | 4.56 | 6.4 | 5.2 | 3.2 |
| 200853_at | “H2A histone family, member Z” | −0.1454 | 1.29 | −0.2888 | −0.1469 | 0.3389 | 1.274 | 0.3976 | −1.025 |
| 201236_s_at | “BTG family, member 2” | −0.006272 | 0.5032 | 0.9142 | −0.1329 | 0.7774 | 0.2717 | −0.3218 | 0.9 |
| 201483_s_at | suppressor of Ty 4 homolog 1 (S. cerevisiae) | 0.6097 | 1.482 | −0.0874 | 1.187 | 1.818 | 0.9257 | 0.2263 | 0.4099 |
| 201487_at | cathepsin C | 0.07473 | −0.138 | −0.7108 | −0.2718 | 0.6703 | 1.105 | 0.9386 | −0.2743 |
| 201890_at | ribonucleotide reductase M2 polypeptide | −0.7399 | 0.9706 | −0.3813 | 0.1577 | 0.621 | 0.8083 | 0.7456 | −5.399 |
| 202095_s_at | baculoviral IAP repeat-containing 5 (survivin) | 0.4877 | 1.084 | −0.3761 | −0.09017 | −0.1052 | 1.08 | 0.7607 | −2.821 |
| 202188_at | KIAA0095 gene product | −1.796 | 2.314 | −1.991 | −1.603 | 1.976 | −0.6295 | −0.9304 | −1.711 |
| 202580_x_at | forkhead box M1 | −0.6508 | 0.6023 | −0.5555 | −1.427 | 0.5444 | 1.37 | −0.08859 | −3.152 |
| 202833_s_at | “serine (or cysteine) proteinase inhibitor, clade A (alpha-1 antiproteinase, antitrypsin), member 1” | 0.2746 | −0.5269 | −1.078 | 2.707 | −0.2308 | −1.418 | −0.1188 | −0.8583 |
| 203362_s_at | MAD2 mitotic arrest deficient-like 1 (yeast) | 0.5555 | 1.23 | 0.3254 | 0.1155 | 0.4871 | 1.438 | 1.036 | −1.972 |
| 203510_at | met proto-oncogene (hepatocyte growth factor receptor) | −0.6627 | 0.2747 | −3.044 | −2.481 | 0.6526 | −4.04 | 0.4437 | −3.992 |
| 203687_at | chemokine (C-X3-C motif) ligand 1 | −0.4788 | −1.04 | −2.797 | −2.23 | −0.6706 | −1.11 | −2.529 | −2.577 |
| 203764_at | “discs, large homolog 7 (Drosophila)” | −0.1027 | 1.189 | −0.2506 | −0.2952 | 0.9748 | 0.2407 | 0.5747 | −0.8813 |
| 204444_at | kinesin-like 1 | 0.4308 | 0.5351 | −1.102 | −0.5121 | 0.639 | 1.578 | 0.6654 | −2.001 |
| 204603_at | exonuclease 1 | −0.1736 | 0.8347 | −0.2122 | 0.388 | 0.3089 | 0.009233 | 0.125 | −0.8951 |
| 204623_at | trefoil factor 3 (intestinal) | 1.455 | −0.4971 | 2.351 | 0.5665 | 1.052 | 0.5084 | 0.5281 | 0.7725 |
| 204766_s_at | nudix (nucleoside diphosphate linked moiety X)-type motif 1 | −0.9492 | −1.983 | −2.618 | −1.673 | −1.818 | 0.5712 | 1.498 | −2.065 |
| 205240_at | LGN protein | −0.7977 | −0.5835 | −1.249 | −1.116 | 1.44 | 2.552 | 1.205 | −1.955 |
| 206110_at | “H3 histone family, member K” | 0.3186 | 0.3631 | −0.3704 | 0.5011 | 2.598 | 2.647 | 1.255 | 0.5199 |
| 206461_x_at | metallothionein 1H | −0.39 | 0.0233 | −1.317 | −1.293 | 0.4777 | 0.4891 | −1.672 | −1.894 |
| 208433_s_at | “low density lipoprotein receptor-related protein 8, apolipoprotein e receptor” | 0.6069 | −0.8708 | −2.261 | 0.1292 | −0.03907 | −0.4007 | −0.3898 | 0.1057 |
| 208546_x_at | “H2B histone family, member J” | −1.122 | 0.8316 | −3.721 | 0.9474 | 2.263 | 2.202 | 0.516 | 0.4872 |
| 208581_x_at | metallothionein 1X | −0.8297 | 0.1821 | −1.11 | −1.774 | 0.7188 | 1 | −1.864 | −1.873 |
| 208767_s_at | lysosomal associated protein transmembrane 4 beta | 0.5525 | 1.335 | 0.1038 | 1.465 | −0.00953 | 0.74 | 0.1544 | 1.263 |
| 209040_s_at | “proteasome (prosome, macropain) subunit, beta type, 8 (large multifunctional protease 7)” | 0.3152 | 0.588 | −3.669 | 0.342 | 1.33 | 1.492 | −3.361 | 0.1566 |
| 209114_at | tetraspan 1 | 0.8555 | −0.07584 | −2.888 | 0.727 | 1.287 | 0.618 | 1.377 | −0.4489 |
| 209398_at | “H1 histone family, member 2” | 0.6575 | 1.078 | −1.211 | 2.027 | 2.668 | 3.317 | 0.775 | −0.02843 |
| 209806_at | “H2B histone family, member T” | 0.04495 | 1.146 | −1.895 | 0.5906 | 1.685 | 1.715 | −0.007187 | 0.05995 |
| 209832_s_at | DNA replication factor | −0.2093 | 0.4368 | −0.6791 | −3.906 | 1.28 | 0.9618 | 0.448 | −2.259 |
| 209924_at | chemokine (C—C motif) ligand 18 (pulmonary and activation-regulated) | 0.003248 | −2.039 | −0.5577 | −1.466 | 0.6619 | −3.446 | −0.2178 | −0.8463 |
| 210052_s_at | chromosome 20 open reading frame 1 | 0.03423 | 0.7286 | 0.4886 | −0.3575 | 1.121 | 0.7662 | 0.7948 | −1.894 |
| 210559_s_at | “cell division cycle 2, G1 to S and G2 to M” | −0.5529 | 0.8735 | −0.2508 | −0.7717 | 1.068 | 0.9656 | 1.144 | −1.567 |
| 210576_at | “cytochrome P450, subfamily IVF, polypeptide 8” | −1.268 | −0.8577 | −1.438 | −1.47 | −0.9421 | −0.8446 | −0.5397 | −1.359 |
| 210792_x_at | CD27-binding (Siva) protein | −0.6963 | −0.7867 | −0.898 | −0.06822 | 0.09006 | 2.469 | −0.1134 | −0.5174 |
| 211456_x_at | “Metallothionein 1H-like protein [Homo sapiens], mRNA sequence” | −1.117 | −0.04123 | −2.591 | −0.6507 | 0.01816 | −0.01816 | −1.794 | −3.482 |
| 212094_at | paternally expressed 10 | −0.672 | −3.155 | −3.211 | 0.9864 | 0.05423 | −1.836 | 2.36 | −1.438 |
| 212141_at | MCM4 minichromosome maintenance deficient 4 (S. cerevisiae) | −0.01048 | 2.045 | 0.6207 | 0.2774 | 1.282 | 1.395 | 1.012 | 0.2859 |
| 212185_x_at | metallothionein 2A | −0.3496 | 0.1074 | −1.298 | −0.8511 | 0.7438 | 0.6982 | −1.544 | −1.558 |
| 212484_at | Mouse Mammary Turmor Virus Receptor homolog 1 | −2.627 | −1.996 | −2.477 | −1.915 | 1.076 | 1.051 | −0.5181 | −3.237 |
| 212613_at | “butyrophilin, subfamily 3, member A2” | 0.3185 | −3.738 | −1.563 | 0.1766 | 1.357 | 0.6323 | −3.472 | −2.94 |
| 213245_at | “Homo sapiens cDNA FLJ30781 fis, clone FEBRA2000874, mRNA sequence” | −1.177 | −0.2654 | 0.3636 | −0.09124 | 0.2931 | −0.01804 | −0.01614 | −0.4445 |
| 213892_s_at | adenine phosphoribosyltransferase | −0.5899 | 0.858 | −1.057 | 0.1746 | 1.372 | 0.4337 | 0.6628 | −0.6729 |
| 214472_at | “H3 histone family, member B” | −0.4576 | 0.9796 | −3.75 | 0.1232 | 2.512 | 2.872 | 0.3387 | 0.1235 |
| 214614_at | homeo box HB9 | 0.3303 | 0.6661 | −4.35 | 0.4488 | −0.9459 | 0.2757 | 0.05505 | −2.666 |
| 214768_x_at | immunoglobulin kappa constant | −1.296 | −1.718 | −1.429 | −1.093 | 0.96 | 0.5704 | −1.006 | −0.6827 |
| 215214_at | immunoglobulin lambda locus | −0.9318 | −1.034 | −0.2254 | −2.812 | 2.236 | −0.6068 | −1.277 | −0.06673 |
| 217165_x_at | “RNA helicase-related protein [Homo sapiens], mRNA sequence” | −0.6751 | 0.4369 | −0.6799 | −1.835 | 0.9058 | 0.8806 | −1.451 | −2.227 |
| 217755_at | hematological and neurological expressed 1 | −0.1186 | 0.2253 | −0.1708 | 0.5365 | −0.3577 | 1.084 | −0.1124 | −1.483 |
| 218350_s_at | “geminin, DNA replication inhibitor” | −0.786 | 0.0005709 | −1.039 | −0.06399 | 1.25 | 2.648 | 0.2315 | −1.411 |
| 218447_at | DC13 protein | −0.3449 | 0.8981 | −1.241 | −0.1253 | 1.392 | −0.008794 | 0.1917 | −1.02 |
| 218542_at | chromosome 10 open reading frame 3 | 0.7738 | 0.8823 | −0.3105 | −0.2223 | 0.2337 | 1.48 | 0.8353 | −3.057 |
| 218875_s_at | F-box only protein 5 | 0.1373 | 0.4371 | −0.2461 | −0.004244 | 0.6533 | 0.6225 | 1.102 | −0.5123 |
| 219061_s_at | DNA segment on chromosome X (unique) 9879 expressed sequence | 0.1001 | 0.403 | −0.3947 | 0.6783 | 1.636 | 1.591 | −0.3655 | −0.254 |
| 219493_at | likely ortholog of mouse Shc SH2-domain binding protein 1 | 0.3727 | 1.409 | −0.6428 | 1.684 | 0.2368 | 0.003738 | 1.107 | −0.5274 |
| 219555_s_at | uncharacterized bone marrow protein BM039 | −0.2336 | 2.786 | −0.567 | −0.4366 | 4.215 | −0.09475 | 2.603 | −1.184 |
| 219650_at | hypothetical protein FLJ20105 | 0.4417 | 0.7085 | −0.9848 | −1.671 | −0.6439 | 0.9707 | 1.205 | −1.405 |
| 220085_at | “helicase, lymphoid-specific” | 1.098 | 2.117 | −0.3944 | −0.1505 | 2.926 | 2.196 | 2.545 | −1.554 |
| 220238_s_at | SBBI26 protein | −0.5356 | −1.869 | 0.2827 | 0.4126 | 1.225 | 1.143 | 0.855 | −0.5087 |
| 221436_s_at | “likely ortholog of mouse gene rich cluster, C8 gene” | −0.0811 | 1.385 | −0.4145 | −1.594 | 0.7122 | 1.042 | −0.1976 | −2.491 |
| 221521_s_at | HSPC037 protein | 0.5415 | 0.9347 | −0.2726 | −1.719 | 2.054 | 0.7876 | 1.801 | −0.1464 |
| 221539_at | eukaryotic translation initiation factor 4E binding protein 1 | −0.07309 | 2.738 | −1.713 | 0.6294 | −0.158 | 1.168 | 0.05806 | −1.008 |
| 222037_at | “Homo sapiens, clone IMAGE: 5270727, mRNA, mRNA sequence” | 0.3059 | 1.569 | 0.3614 | 0.8596 | 1.005 | 1.397 | 0.742 | −2.44 |

| UID | 20020051 | 20020056 | 980197 | 980261 | 980391 | 2000768 | 2000779 | 990123 | 2000422 | 2000683 | 2000775 | 2000804 | 980346 | 980383 | 990082 | 980177 |
|---|---|---|---|---|---|---|---|---|---|---|---|---|---|---|---|---|
| NPI | 6 | 3.4 | 5.6 | 3.3 | 3.4 | 4.8 | 5.1 | 7.1 | 6.26 | 3.7 | 3.5 | 6.8 | 4.4 | 3.6 | 4.68 | 5.52 |
| 200853_at | 0.7639 | −0.7213 | 1.395 | −0.5183 | −0.1454 | 1.481 | 1.149 | 1.102 | 1.105 | −0.9016 | −0.2015 | 0.6147 | 0.9351 | 0.3702 | −0.78 | 0.7502 |
| 201236_s_at | −0.4893 | 2.126 | −0.2778 | 1.747 | 1.955 | 0.1703 | −0.09297 | −0.8116 | 0.2803 | 1.573 | −0.4571 | 0.2552 | 0.5244 | 0.8867 | 0.5263 | 0.278 |
| 201483_s_at | 0.7686 | −0.174 | 0.603 | −0.8021 | 0.4711 | 0.8151 | 1.052 | 0.8867 | 0.6619 | −0.7083 | −0.6522 | 1.6 | −1.372 | −0.8661 | −1.684 | 1.396 |
| 201487_at | 0.9838 | −0.7759 | 0.2844 | −1.244 | −0.8704 | 2.864 | −1.201 | 1.285 | 0.546 | −0.9224 | 1.085 | 0.1034 | −0.2643 | −1.447 | −1.158 | 1.502 |
| 201890_at | 0.7672 | −1.632 | −0.244 | −0.8654 | −0.1484 | 0.5024 | 0.8568 | 1.374 | −0.2848 | −1.812 | 0.2609 | 1.347 | 0.729 | −0.3775 | −2.774 | −0.1699 |
| 202095_s_at | 2.266 | −4.419 | −0.4761 | 0.824 | 0.1905 | 0.7446 | 0.1633 | 1.969 | 1.562 | −1.104 | 0.04571 | 0.743 | 0.8446 | −0.009848 | −3.205 | 1.153 |
| 202188_at | 0.679 | −1.065 | 1.582 | −1.575 | −0.9511 | 0.02638 | 1.178 | 0.9636 | 1.625 | 0.2826 | −0.007729 | −1.634 | −0.9197 | −1.993 | −1.106 | 2.364 |
| 202580_x_at | 0.4569 | −2.362 | 0.1443 | 0.08023 | −0.4678 | 0.585 | 0.957 | 0.422 | 0.7627 | −0.8553 | −0.333 | 2.135 | 0.8166 | 0.2332 | −3.414 | 0.3564 |
| 202833_s_at | −0.9274 | −0.6578 | 1.595 | −2.001 | −0.7663 | 0.5393 | −1.519 | 4.702 | 3.99 | −1.54 | −0.02002 | −0.155 | −0.2741 | −0.7656 | −1.361 | 1.912 |
| 203362_s_at | 1.544 | −0.8522 | 0.8364 | 0.1556 | −0.1722 | 1.08 | 0.6537 | 0.6816 | 0.5234 | −1.458 | −0.7681 | 1.186 | 0.6807 | 0.2946 | −1.65 | −0.08355 |
| 203510_at | 1.427 | −2.922 | 0.8314 | −1.178 | −2.988 | −3.458 | −3.411 | −5.32 | −0.736 | −2.917 | −2.182 | −3.039 | −0.05372 | −3.493 | −2.429 | −1.026 |
| 203687_at | −0.4576 | −2.239 | −0.3712 | −2.375 | −2.768 | 0.2528 | 1.41 | −1.073 | −1.986 | −2.184 | −2.261 | −2.136 | −0.07089 | −2.5 | −1.205 | −1.83 |
| 203764_at | 1.27 | −1.848 | 0.4207 | −0.518 | −0.04025 | 0.3163 | 0.2513 | 0.8161 | 0.02686 | −0.9493 | −0.1979 | 1.205 | 0.8268 | 0.3441 | −1.558 | 0.5411 |
| 204444_at | 1.489 | −1.467 | 0.6428 | 0.01471 | −0.2197 | 0.3222 | 1.165 | 1.213 | −0.3525 | −0.9507 | 0.4009 | 1.87 | 0.6619 | 0.7972 | −1.251 | 0.1869 |
| 204603_at | 0.4739 | −1.07 | 0.2971 | −1.526 | −0.7977 | 0.3296 | 0.3566 | 0.4979 | 0.802 | −0.3959 | −1.22 | 0.3622 | 1.07 | −0.7394 | −1.399 | −0.1352 |
| 204623_at | 1.962 | 0.3898 | 4.402 | 1.319 | 0.2033 | 0.532 | 1.723 | 1.79 | −0.485 | −1.366 | −0.124 | 5.559 | 1.444 | −0.2338 | −1.252 | 1.742 |
| 204766_s_at | −0.03667 | −1.965 | 1.477 | 1.079 | −1.462 | −0.75 | 1.233 | 0.7507 | −1.709 | −1.337 | −0.2776 | 0.4907 | 0.6079 | −1.635 | −2.172 | 2.303 |
| 205240_at | 3.574 | −1.816 | −1.346 | −1.502 | 2.445 | −0.6427 | 1.822 | 2.117 | 3.053 | −1.752 | −1.022 | 2.116 | 1.297 | 1.744 | 0.07903 | 1.427 |
| 206110_at | 2 | 1.18 | −0.5388 | −0.9876 | −3.643 | 2.091 | 3.928 | −0.8569 | −0.6761 | −1.389 | 0.4854 | 0.2935 | 1.198 | −1.017 | 0.2135 | 0.1006 |
| 206461_x_at | −0.8382 | −0.7782 | −0.9382 | −0.3932 | −0.6041 | 0.3568 | 0.1575 | 0.1177 | 1.625 | −3.82 | −0.3759 | 0.2691 | 0.253 | −0.6888 | −1.201 | −0.5355 |
| 208433_s_at | 0.2285 | −0.1193 | −0.4589 | −1.55 | −1.479 | 0.43 | 0.9039 | −0.3572 | −0.3884 | −1.098 | −2.824 | 0.1473 | 0.2335 | −0.3116 | −1.666 | −1.003 |
| 208546_x_at | 1.573 | 0.5561 | 0.9048 | −3.716 | −0.8904 | 1.762 | 2.578 | −0.893 | −0.3691 | −0.382 | 0.7664 | 1.389 | 1.052 | −2.973 | 0.7082 | 0.2382 |
| 208581_x_at | −0.8671 | −1.706 | −0.8531 | −0.5323 | −0.8031 | 0.6624 | 0.772 | −0.01007 | 1.515 | −1.761 | −0.4597 | −0.2998 | 0.2791 | −1.453 | −1.127 | 0.01007 |
| 208767_s_at | 1.142 | −1.518 | 0.8536 | −0.5525 | −1.175 | 0.919 | 0.3282 | 1.298 | 2.165 | −1.381 | −0.9267 | −0.2713 | 0.4081 | 0.7598 | −1.833 | 1.039 |
| 209040_s_at | 0.0934 | −1.052 | 0.5683 | −4.269 | −0.4344 | 1.387 | −0.772 | 1.844 | 0.7642 | −0.7578 | 1.344 | 0.897 | 0.5398 | −2.669 | −0.7838 | 1.814 |
| 209114_at | −0.4048 | −0.05417 | 1.687 | 0.3291 | −0.4002 | −0.4801 | 2.494 | 1.825 | 2.19 | 1.306 | −1.028 | 0.923 | −0.6499 | 1.26 | −1.03 | 1.344 |
| 209398_at | 2.147 | 0.8804 | 0.3597 | −1.194 | −0.7188 | 3.047 | 3.613 | −0.5534 | −0.2921 | −0.7163 | 1.459 | 0.1181 | 2.258 | −1.025 | 1.194 | 1.332 |
| 209806_at | 1.35 | −0.4676 | 1.172 | −0.5088 | −1.877 | 1.592 | 2.085 | −0.9202 | −1.274 | −0.3533 | 1.334 | 0.1805 | 1.377 | −1.049 | 0.6906 | 0.3721 |
| 209832_s_at | 1.283 | −0.784 | 0.785 | −0.3879 | −0.2683 | 0.792 | 1.165 | 0.8799 | 0.953 | −0.4351 | 0.2743 | 1.248 | 1.03 | −0.5027 | −1.445 | 0.5995 |
| 209924_at | 1.369 | −1.233 | 0.7177 | −0.5422 | −1.256 | 0.8261 | −0.4693 | 0.8621 | 0.746 | −0.07244 | 0.1775 | 0.7399 | −0.003248 | −3.226 | −0.8553 | 0.7689 |
| 210052_s_at | 1.671 | −1.204 | −0.1785 | −0.3447 | −0.0865 | 0.7136 | 0.9764 | 1.282 | 1.064 | −1.092 | −0.1238 | 1.548 | 0.5728 | 0.244 | −1.466 | 1.214 |
| 210559_s_at | 1.246 | −1.044 | 0.6194 | −0.3937 | −0.2029 | 0.3902 | 1.055 | 1.288 | 1.092 | −2.252 | −0.06658 | 2.025 | 0.7638 | 0.6873 | −1.305 | 0.6587 |
| 210576_at | −0.6011 | 3.761 | −0.9546 | 3.704 | 4.427 | −0.8408 | −0.8623 | 1.306 | −1.195 | 0.05855 | 9.803 | −0.3689 | −0.5783 | 5.219 | 2.246 | −0.02554 |
| 210792_x_at | 1.706 | −0.2906 | 0.323 | −1.999 | 0.2819 | 2.967 | 0.3527 | 2.022 | 1.753 | −0.4607 | 0.6391 | 0.2633 | −0.6831 | −1.031 | −2.234 | 2.18 |
| 211456_x_at | −1.126 | −3.411 | −0.8509 | −1.1 | −2.47 | 0.43 | 0.1692 | 0.4534 | 1.738 | −2.633 | −1.199 | 0.5589 | 0.1271 | −1.565 | −1.591 | −0.5746 |
| 212094_at | 1.204 | −2.245 | −1.56 | −3.69 | −2.33 | −0.05982 | −5.556 | −4.073 | 1.828 | −0.1291 | 0.8363 | −2.39 | −0.2446 | −3.414 | 2.494 | 2.876 |
| 212141_at | 2.278 | −2.371 | 0.9675 | −3.468 | 0.08903 | 1.907 | 0.5054 | 1.39 | 1.754 | −0.05179 | 0.1601 | 1.165 | 1.149 | 0.05549 | −0.7758 | 1.347 |
| 212185_x_at | −0.8231 | −1.389 | −0.6833 | −0.213 | −0.4286 | 0.8156 | 0.5282 | −0.1693 | 1.705 | −2.072 | −0.1536 | −0.08535 | 0.3755 | −1.32 | −0.8457 | 0.08054 |
| 212484_at | −1.786 | −0.2226 | 0.05274 | −2.979 | 0.01185 | 0.3771 | 0.2132 | 0.9695 | 0.4707 | −0.3862 | −0.02294 | 0.7631 | 0.4243 | −2.427 | −0.2257 | 0.1713 |
| 212613_at | −0.948 | −2.16 | −0.9575 | −2.186 | −3.444 | 0.9747 | −0.4156 | −0.1155 | −2.642 | −0.3187 | 1.238 | 0.4254 | −1.869 | −0.5386 | −0.05145 | 0.4147 |
| 213245_at | 0.6931 | 0.1661 | 0.3127 | 0.06183 | 0.3379 | −0.3904 | 0.3865 | 1.577 | 0.5969 | −0.228 | −0.449 | 2.261 | −0.01159 | −0.1097 | −0.09017 | 2.64 |
| 213892_s_at | 0.1435 | −0.8664 | 0.9885 | 0.2662 | −0.3091 | 0.342 | 0.8098 | 0.5261 | 0.3701 | −0.9586 | −0.3689 | 0.01619 | 0.5974 | −0.7273 | 0.04012 | 1.121 |
| 214472_at | 1.964 | 1.03 | 0.2118 | −3.128 | 0.6922 | 1.271 | 3.664 | −0.3823 | −2.636 | −1.03 | 0.4435 | 1.095 | 0.8844 | −0.1423 | 1.02 | −0.1162 |
| 214614_at | 2.021 | −3.871 | 0.9336 | −0.2894 | −1.361 | 1.553 | 1.158 | 1.157 | 0.008503 | −2.796 | −0.2623 | 0.4189 | 0.2761 | −3.09 | −3.685 | −3.661 |
| 214768_x_at | 3.806 | 0.2901 | 1.207 | −5.152 | 0.4289 | 3.816 | 0.1494 | −1.782 | 1.674 | 0.05538 | 1.724 | 2.769 | −2.438 | −3.058 | −1.8 | −0.4986 |
| 215214_at | 4.293 | 0.79 | 4.235 | −5.33 | −1.887 | 6.318 | −0.6783 | −0.7629 | 0.2533 | 0.7153 | 3.115 | −0.8651 | 3.404 | −2.903 | −0.6081 | −0.107 |
| 217165_x_at | −0.8228 | −1.369 | −0.5914 | −1.336 | −0.7548 | 0.2035 | 0.7183 | 0.6163 | 1.399 | −4.079 | −0.6624 | 0.7245 | −0.6689 | −1.315 | −1.109 | 0.4527 |
| 217755_at | 1.265 | −0.5934 | −0.03318 | −1.268 | −0.6899 | 1.374 | 2.792 | 1.898 | 0.5077 | −0.08982 | 0.241 | −0.07873 | 1.565 | 0.009169 | −0.1982 | 1.525 |
| 218350_s_at | 2.161 | −1.703 | −0.2055 | −0.9262 | −0.9141 | 0.1189 | 0.6987 | 1.083 | −0.08697 | −0.9574 | 0.1942 | −0.04529 | 0.3457 | −0.2563 | −1.103 | −0.1227 |
| 218447_at | 0.8442 | −0.7655 | 1.281 | 0.4179 | −0.6047 | 0.707 | 0.629 | 0.4928 | 0.3587 | −0.815 | −0.2259 | 0.153 | −0.2051 | −0.9587 | −1.194 | 0.1652 |
| 218542_at | 1.639 | −2.889 | 0.9011 | 0.01222 | 0.5653 | 0.3149 | 0.9069 | 0.7584 | 0.9998 | −1.406 | 0.6297 | 1.792 | 1.085 | 0.4355 | −1.843 | −0.01727 |
| 218875_s_at | 2.342 | −0.7599 | 0.6561 | −0.7147 | −0.04562 | 1.172 | 0.3008 | 1.113 | 0.6165 | −0.5506 | 0.006706 | 0.7875 | 0.6787 | 0.2788 | −0.7371 | 0.5419 |
| 219061_s_at | 0.3603 | −0.1915 | 0.8592 | −1.293 | 0.2111 | 2.012 | 0.7631 | 1.2 | 0.6261 | −0.5851 | 0.02172 | 0.1617 | 1.074 | −0.05658 | 0.2486 | 1.981 |
| 219493_at | 1.644 | −0.08562 | −1.648 | −0.1838 | −0.6264 | 0.3935 | 0.02768 | 2.026 | −0.4948 | −0.5828 | 0.05786 | 0.282 | 1.483 | 0.2727 | −1.421 | 0.4901 |
| 219555_s_at | 3.628 | −0.9118 | 3.769 | 0.1243 | −0.8211 | 0.06107 | 0.2847 | 2.663 | 3.131 | −0.5802 | 1.592 | 3.039 | 0.03131 | 2.418 | −0.6508 | 0.9633 |
| 219650_at | 1.856 | −1.188 | 0.8473 | −1.826 | −0.0391 | 0.3461 | 0.911 | 1.13 | 1.107 | −0.7248 | 0.7535 | 1.691 | −0.3078 | 0.7382 | −1.113 | 1.668 |
| 220085_at | 2.94 | −0.6154 | −0.6666 | −1.443 | 0.9973 | 1.975 | 3.076 | 3.464 | 2.717 | −0.6921 | −0.3206 | −0.6188 | 2.428 | 2.27 | −1.005 | 0.9854 |
| 220238_s_at | 0.01481 | −2.392 | −0.7889 | 0.06984 | −1.5 | −0.04597 | −0.1081 | −0.1832 | −0.02539 | −4.754 | −0.8441 | −0.2432 | 1.218 | −0.8203 | −4.598 | 1.751 |
| 221436_s_at | 0.9193 | −2.686 | 1.146 | 0.3385 | −0.1127 | 1.256 | 0.8854 | 0.3978 | 1.538 | −0.1481 | 0.1661 | 2.272 | 0.9145 | 0.5171 | −2.271 | 1.161 |
| 221521_s_at | 2.363 | −3.6 | 1.422 | 1.45 | −0.04945 | 0.04416 | 2.338 | 2.103 | 2.45 | −1.793 | 0.377 | 2.219 | 0.3689 | 0.1082 | −0.2741 | 1.391 |
| 221539_at | −0.1794 | −1.094 | −0.09421 | −0.7066 | −0.399 | 1.699 | 0.229 | 3.751 | 0.7476 | −0.6211 | −0.8323 | 0.4994 | −0.2218 | −0.5206 | 0.08783 | 1.111 |
| 222037_at | 1.361 | −3.674 | 1.354 | −1.237 | 0.4627 | 1.613 | 0.441 | 0.8522 | 1.526 | −0.2248 | 0.9008 | 1.122 | 1.145 | 0.03042 | 0.09205 | 0.326 |

| UID | 980178 | 980403 | 980434 | 990075 | 990113 | 990107 | 980208 | 980220 | 980221 | 990375 |
|---|---|---|---|---|---|---|---|---|---|---|
| NPI | 5.74 | 4.6 | 4.6 | 6.5 | 7.8 | 3.8 | 6.5 | 3.74 | 6.3 | 2.3 |
| 200853_at | −0.1024 | 1.684 | 0.4969 | 0.5195 | −0.319 | 0.1196 | 0.7886 | −0.002354 | −0.2928 | −0.0726 |
| 201236_s_at | 0.6472 | 1.158 | 0.1387 | −0.09749 | 0.4156 | 1.328 | 0.4434 | 1.355 | 0.3473 | 0.866 |
| 201483_s_at | 0.4893 | 1.347 | −0.3128 | −0.5101 | −0.09044 | 0.4318 | 2.904 | 0.4475 | −0.3917 | 0.01991 |
| 201487_at | 0.4309 | 0.9151 | −0.6552 | 1.012 | −0.6763 | −1.624 | 1.46 | −0.292 | −0.01074 | −0.688 |
| 201890_at | −0.3712 | 0.3715 | 0.09703 | −0.7396 | −0.327 | −0.01902 | 0.8041 | −0.9266 | −2.221 | −1.183 |
| 202095_s_at | −0.6422 | 2.755 | −0.519 | 0.6679 | 0.3284 | −0.1171 | 0.3173 | −0.6899 | −0.7857 | −0.5155 |
| 202188_at | 2.902 | 1.597 | 2.523 | 2.11 | −1.844 | −0.00351 | 1.418 | −0.7783 | 2.405 | −1.969 |
| 202580_x_at | 0.2976 | 0.2955 | −0.1124 | 0.1875 | 0.1209 | −0.427 | 0.719 | −0.4973 | −1.537 | −1.016 |
| 202833_s_at | 1.678 | −0.154 | 2.179 | 2.225 | 4.768 | −0.2613 | 0.8987 | 2.563 | 2.903 | −1.034 |
| 203362_s_at | −0.8345 | 1.333 | 0.09134 | −0.4979 | −0.4036 | −0.4871 | 0.6176 | −0.915 | −1.052 | −1.223 |
| 203510_at | 1.1 | −2.892 | −2.466 | 0.4109 | −0.7238 | −4.735 | −3.73 | −0.0802 | 1.504 | −4.287 |
| 203687_at | −1.828 | −0.1391 | −2.839 | −1.253 | −1.423 | 0.2924 | 1.175 | 0.01629 | 0.8783 | −2.518 |
| 203764_at | −0.6736 | 1.42 | −0.04316 | 0.5578 | 0.1168 | −0.4682 | 0.3234 | −1.2 | −0.7031 | −1.168 |
| 204444_at | −0.7514 | 0.7181 | 0.312 | 0.1993 | 0.6377 | −0.3319 | 0.7199 | −0.7578 | −0.4692 | −0.7489 |
| 204603_at | −0.4691 | 1.155 | −0.1053 | −0.1224 | 0.6709 | −0.4958 | 0.124 | −0.2691 | −0.1738 | −0.4979 |
| 204623_at | 1.662 | 1.694 | 3.888 | 1.79 | 2.073 | −1.016 | 2.829 | 2.656 | 0.7808 | −1.66 |
| 204766_s_at | 2.477 | 1.268 | 1.464 | 2.17 | 0.9556 | −0.7158 | 1.903 | 0.1623 | −1.271 | −2.434 |
| 205240_at | −0.6868 | 1.697 | −0.5062 | −0.07903 | 2.346 | 0.874 | 1.947 | −2.268 | 0.2659 | 1.408 |
| 206110_at | 0.2619 | −0.5187 | −0.8164 | −0.6088 | 1.905 | 1.035 | 3.605 | −1.59 | 1.022 | −0.04912 |
| 206461_x_at | 1.776 | 0.261 | −0.1324 | −1.831 | −0.2725 | −1.273 | 0.5747 | 0.08164 | 0.5835 | −1.239 |
| 208433_s_at | −0.1305 | 0.7345 | −3.906 | −0.09776 | −2.038 | −3.61 | −0.2532 | −2.656 | −1.167 | −2.498 |
| 208546_x_at | 1.351 | −0.3061 | 0.4062 | 0.2047 | 1.654 | 1.308 | 2.345 | 0.5585 | 1.121 | 0.5644 |
| 208581_x_at | 1.767 | 0.4699 | 0.1739 | −1.292 | −0.4647 | −0.5576 | 0.7377 | 0.3646 | 0.9934 | −1.452 |
| 208767_s_at | 0.07857 | 0.4824 | −0.4325 | 3.175 | −0.652 | −0.03558 | −0.2332 | 0.3561 | 0.7377 | −1.186 |
| 209040_s_at | 1.461 | 0.9016 | 0.5718 | 1.511 | 0.2541 | 0.4053 | 2.276 | 0.9434 | 0.5052 | −1.327 |
| 209114_at | 1.158 | 0.9383 | 3.609 | −0.4017 | 0.3422 | −0.6132 | 0.7248 | 0.5805 | 2.243 | −1.668 |
| 209398_at | 1.659 | −0.6719 | 1.729 | 0.1807 | 0.586 | 1.717 | 3.717 | 0.9074 | 1.742 | 0.5849 |
| 209806_at | 1.173 | −0.003976 | 0.04341 | −0.4338 | 0.003975 | 0.4399 | 1.955 | 0.2045 | 0.4444 | −0.08685 |
| 209832_s_at | −0.5054 | 1.063 | −0.009595 | −0.244 | −0.3794 | −0.1792 | 0.2804 | −1.287 | −0.2545 | −0.2157 |
| 209924_at | −0.2968 | 0.6344 | −1.833 | 0.8685 | −2.811 | −3.967 | 0.8904 | −1.524 | −0.7571 | −0.8797 |
| 210052_s_at | 0.08015 | 1.559 | 0.1766 | −0.2576 | 0.3938 | 0.1252 | 1.078 | −0.47 | −0.636 | −0.7279 |
| 210559_s_at | −0.9514 | 1.169 | 0.4542 | 0.02187 | 0.7038 | 0.1117 | 0.3919 | 0.1868 | 0.1606 | −0.3175 |
| 210576_at | 0.2243 | −0.5602 | 0.0802 | −0.842 | 0.004669 | 2.291 | 0.9273 | −1.093 | −1.182 | 4.035 |
| 210792_x_at | 1.734 | 0.7072 | 0.2691 | 1.524 | 0.3786 | 0.8489 | 1.1 | −0.4151 | 0.8751 | −0.08786 |
| 211456_x_at | 1.378 | 0.1608 | −0.1149 | −3.006 | −0.9624 | −0.8765 | 0.5023 | 0.03099 | 0.7018 | −2.313 |
| 212094_at | −0.3911 | 2.367 | 3.409 | 0.03678 | 2.613 | −1.918 | −0.8758 | −2.307 | 2.694 | 2.508 |
| 212141_at | 0.5721 | 2.048 | 0.1982 | 0.2689 | 0.3733 | 0.6741 | 0.8496 | −2.634 | 0.1973 | −2.22 |
| 212185_x_at | 1.222 | 0.4245 | 0.2713 | −1.085 | −0.3512 | −0.3473 | 07846 | 0.1873 | 0.5453 | −1.284 |
| 212484_at | 1.835 | 0.2063 | 1.064 | −0.05172 | 0.6024 | 0.3308 | −0.9625 | 0.3044 | 1.666 | −1.209 |
| 212613_at | 1.095 | 0.8142 | 0.03735 | 1.204 | −0.5544 | −2.84 | 1.699 | −0.8163 | 0.5606 | −0.9554 |
| 213245_at | 1.719 | 0.0225 | 2.535 | 2.159 | 3.16 | −0.7841 | −0.1771 | 0.2709 | 1.316 | −0.02205 |
| 213892_s_at | 1.028 | 1.872 | 0.01646 | 0.3821 | −0.3695 | −0.267 | 1.251 | 0.0724 | 0.7799 | −0.4139 |
| 214472_at | 1.411 | 0.0753 | 0.02653 | −0.948 | 1.585 | 0.251 | 2.84 | 0.02776 | 0.06318 | 0.6362 |
| 214614_at | −2.847 | −1.842 | −1.004 | −1.165 | 0.07506 | −2.92 | 1.236 | −0.5473 | −1.549 | −1.041 |
| 214768_x_at | 0.3274 | −1.575 | −1.786 | 3.895 | −3.171 | −3.169 | 3.938 | −1.158 | 2.259 | −2.557 |
| 215214_at | 1.506 | 0.2801 | −2.253 | 0.8081 | −1.608 | −3.416 | 5.034 | −0.6629 | 2.871 | −3.191 |
| 217165_x_at | 2.207 | 0.4983 | 0.6566 | −1.657 | −0.2512 | −0.3176 | 0.6302 | 0.0875 | 1.176 | −1.656 |
| 217755_at | 0.4835 | 1.834 | −0.454 | 0.2932 | −0.6486 | 0.209 | 2.066 | 0.4042 | −0.04373 | −0.1996 |
| 218350_s_at | −0.4606 | −0.07571 | −1.061 | −0.3062 | −0.3259 | −0.8389 | 0.4498 | −0.8626 | −0.8218 | −0.5508 |
| 218447_at | 0.3473 | 1.707 | 0.1991 | 0.8604 | −0.3649 | −0.1633 | 1.053 | −0.04051 | 0.6493 | −0.7617 |
| 218542_at | −0.6697 | 0.7572 | −0.1458 | 0.0329 | −0.2066 | −0.384 | 0.9562 | −1.544 | −0.2706 | −0.3654 |
| 218875_s_at | −0.9452 | 0.9993 | −0.3711 | 0.8086 | −0.6675 | −0.4852 | 0.5996 | −0.8644 | −2.121 | −1.063 |
| 219061_s_at | 1.841 | 1.55 | 0.5562 | −0.8898 | −0.3676 | 0.07326 | 0.927 | 0.4476 | −0.0437 | −0.3224 |
| 219493_at | −0.9642 | 1.269 | 0.1536 | −0.1922 | −0.1069 | −0.6264 | 1.013 | −0.3574 | −0.8402 | −2.008 |
| 219555_s_at | 1.56 | 4.613 | −0.07838 | 3.06 | −0.7089 | −0.2662 | 0.3197 | −0.9291 | 2.801 | −1.049 |
| 219650_at | 0.1232 | 0.8186 | 0.5107 | 0.2778 | 0.2186 | −0.4108 | −0.2544 | −1.474 | −0.4748 | −0.7022 |
| 220085_at | −0.4835 | 2.768 | 2.083 | −0.9158 | −0.3327 | −0.8196 | 3.488 | −1.969 | −0.8297 | −0.0946 |
| 220238_s_at | −0.1621 | 0.3076 | 0.3174 | −0.1157 | −0.4725 | −3.034 | −4.345 | −3.132 | 1.094 | −0.5442 |
| 221436_s_at | 1.425 | 1.627 | 1.09 | 1.094 | 0.0009523 | −0.1412 | 0.9679 | −0.7813 | −1.46 | −1.893 |
| 221521_s_at | 1.063 | 3.74 | 1.458 | 1.522 | 1.135 | 0.5133 | 2.025 | 0.005946 | 0.3726 | −0.2652 |
| 221539_at | 4.2 | 0.9997 | 0.1046 | −1.671 | −0.9075 | −0.3762 | 2.44 | −0.2567 | 0.1609 | −0.2192 |
| 222037_at | 0.3462 | 1.786 | −0.4235 | 1.052 | −0.2547 | −0.4347 | 0.1949 | 0.04239 | −0.1561 | −0.5869 |

TABLE S15
Weighted Voting parameters for mean (μ) and standard deviation (σ) of expression data for genes of the prognostic set

| | | Low-NPI | | High-NPI | |
|---|---|---|---|---|---|
| Probe_ID | Gene Name | mean | SD | mean | SD |
| 213892_s_at | adenine phosphoribosyltransferase | −0.4139 | 0.419865 | 0.5261 | 0.5756 |
| 212141_at | MCM4 minichromosome maintenance deficient 4 (S. cerevisiae) | 0.05549 | 1.527753 | 1.012 | 0.771858 |
| 204603_at | exonuclease 1 | −0.7394 | 0.414899 | 0.3089 | 0.546392 |
| 211456_x_at | Metallothionein 1H-like protein [Homo sapiens], mRNA sequence | −2.313 | 1.10771 | −0.01816 | 1.061529 |
| 222037_at | Homo sapiens, clone IMAGE: 5270727, mRNA, mRNA sequence | −0.2248 | 1.360941 | 0.8596 | 0.648812 |
| 218447_at | DC13 protein | −0.7617 | 0.497934 | 0.3587 | 0.655529 |
| 221521_s_at | HSPC037 protein | −0.04945 | 1.328055 | 1.422 | 1.13546 |
| 200853_at | H2A histone family, member Z | −0.2015 | 0.437181 | 0.7502 | 0.667011 |
| 203764_at | discs, large homolog 7 (Drosophila) | −0.518 | 0.626375 | 0.3234 | 0.711794 |
| 217165_x_at | RNA helicase-related protein [Homo sapiens], mRNA sequence | −1.315 | 1.126665 | 0.4527 | 1.042786 |
| 204444_at | kinesin-like 1 | −0.7489 | 0.817308 | 0.6377 | 0.760632 |
| 210052_s_at | chromosome 20 open reading frame 1 | −0.3447 | 0.713083 | 0.7286 | 0.785951 |
| 202188_at | KIAA0095 gene product | −1.065 | 0.858421 | 1.178 | 1.616733 |
| 220085_at | helicase, lymphoid-specific | −0.6154 | 1.198542 | 2.083 | 1.619802 |
| 214614_at | homeo box HB9 | −2.666 | 1.462508 | 0.2757 | 1.583945 |
| 219061_s_at | DNA segment on chromosome X (unique) 9879 expressed sequence | −0.1915 | 0.461491 | 0.6783 | 0.795975 |
| 203362_s_at | MAD2 mitotic arrest deficient-like 1 (yeast) | −0.7681 | 0.74839 | 0.6176 | 0.842842 |
| 221539_at | eukaryotic translation initiation factor 4E binding protein 1 | −0.6211 | 0.442172 | 0.229 | 1.408505 |
| 201487_at | cathepsin C | −0.7759 | 0.729779 | 0.4309 | 0.950128 |
| 208546_x_at | H2B histone family, member J | 0.4872 | 1.894009 | 0.9474 | 1.009994 |
| 209040_s_at | proteasome (prosome, macropain) subunit, beta type, 8 | −0.7578 | 1.8346 | 0.588 | 1.159099 |
| | (large multifunctional protease 7) | | | | |
| 219650_at | hypothetical protein FLJ20105 | −0.7248 | 0.85837 | 0.5107 | 0.893847 |
| 218542_at | chromosome 10 open reading frame 3 | −0.3654 | 1.305871 | 0.7584 | 0.82541 |
| 219555_s_at | uncharacterized bone marrow protein BM039 | −0.5802 | 1.164774 | 1.56 | 1.763962 |
| 221436_s_at | likely ortholog of mouse gene rich cluster, C8 gene | −0.1481 | 1.137308 | 0.9679 | 1.10724 |
| 210559_s_at | cell division cycle 2, G1 to S and G2 to M | −0.2508 | 0.844298 | 0.7038 | 0.805354 |
| 212185_x_at | metallothionein 2A | −1.284 | 0.725732 | 0.1074 | 0.798804 |
| 218350_s_at | geminin, DNA replication inhibitor | −0.9141 | 0.51298 | −0.06399 | 0.926376 |
| 208433_s_at | low density lipoprotein receptor-related protein 8, | −1.55 | 1.219961 | −0.2532 | 1.04719 |
| | apolipoprotein e receptor | | | | |
| 217755_at | hematological and neurological expressed 1 | −0.1708 | 0.614723 | 0.4835 | 0.951001 |
| 209398_at | H1 histone family, member 2 | −0.02843 | 1.093238 | 1.332 | 1.299819 |
| 204766_s_at | nudix (nucleoside diphosphate linked moiety X)-type motif 1 | −1.462 | 1.152307 | 0.6079 | 1.516876 |
| 208581_x_at | metallothionein 1X | −1.11 | 0.696985 | 0.1739 | 0.997649 |
| 209806_at | H2B histone family, member T | −0.3533 | 0.961244 | 0.5906 | 0.913624 |
| 209114_at | tetraspan 1 | −0.4002 | 1.24355 | 0.923 | 1.133855 |
| 206461_x_at | metallothionein 1H | −0.7782 | 1.051675 | 0.1177 | 0.916536 |
| 206110_at | H3 histone family, member K | −0.3704 | 1.40578 | 0.3631 | 1.411458 |
| 201890_at | ribonucleotide reductase M2 polypeptide | −0.8654 | 1.559316 | 0.3715 | 1.024143 |
| 202095_s_at | baculoviral IAP repeat-containing 5 (survivin) | −0.3761 | 1.515513 | 0.6679 | 1.21519 |
| 218875_s_at | F-box only protein 5 | −0.5123 | 0.409105 | 0.6165 | 0.900364 |
| 202833_s_at | serine (or cysteine) proteinase inhibitor, clade A | −0.7663 | 1.176481 | 0.5393 | 1.901084 |
| | (alpha-1 antiproteinase, antitrypsin), member 1 | | | | |
| 208767_s_at | lysosomal associated protein transmembrane 4 beta | −0.5525 | 0.938047 | 0.5525 | 1.011665 |
| 203687_at | chemokine (C-X3-C motif) ligand 1 | −2.375 | 1.081471 | −1.073 | 1.154088 |
| 210792_x_at | CD27-binding (Siva) protein | −0.4151 | 0.800901 | 0.3786 | 1.230555 |
| 205240_at | LGN protein | −1.249 | 1.72051 | 1.297 | 1.446916 |
| 212484_at | Mouse Mammary Turmor Virus Receptor homolog 1 | −0.3862 | 1.394896 | 0.2132 | 1.187908 |
| 202580_x_at | forkhead box M1 | −0.4973 | 1.022497 | 0.3564 | 1.104339 |
| 203510_at | met proto-oncogene (hepatocyte growth factor receptor) | −2.988 | 1.352621 | −0.736 | 2.009295 |
| 212613_at | butyrophilin, subfamily 3, member A2 | −1.563 | 1.383434 | 0.1766 | 1.475442 |
| 220238_s_at | SBBI26 protein | −0.8441 | 1.574483 | −0.04597 | 1.556341 |
| 219493_at | likely ortholog of mouse Shc SH2-domain binding protein 1 | −0.5274 | 0.594225 | 0.282 | 1.007135 |
| 214472_at | H3 histone family, member B | 0.1235 | 1.581567 | 0.8844 | 1.40927 |
| 204623_at | trefoil factor 3 (intestinal) | 0.2033 | 1.408904 | 1.662 | 1.554202 |
| 215214_at | immunoglobulin lambda locus | −0.6629 | 2.409822 | −0.107 | 2.500735 |
| 209832_s_at | DNA replication factor | −0.4351 | 0.674077 | 0.5995 | 1.153719 |
| 213245_at | Homo sapiens cDNA FLJ30781 fis, clone FEBRA2000874, | −0.02205 | 0.369593 | 0.3127 | 1.16657 |
| | mRNA sequence | | | | |
| 209924_at | chemokine (C—C motif) ligand 18 (pulmonary and activation-regulated) | −0.8797 | 1.267438 | 0.003248 | 1.311969 |
| 214768_x_at | immunoglobulin kappa constant | −1.158 | 1.997589 | 0.1494 | 2.246666 |
| 201483_s_at | suppressor of Ty 4 homolog 1 (S. cerevisiae) | −0.0874 | 0.541135 | 0.7686 | 1.030094 |
| 212094_at | paternally expressed 10 | −2.245 | 1.918298 | 0.03678 | 2.405576 |
| 201236_s_at | BTG family, member 2 | 1.328 | 0.70948 | 0.2717 | 0.438693 |
| 210576_at | cytochrome P450, subfamily IVF, polypeptide 8 | 3.704 | 3.447008 | −0.6011 | 0.891116 |

TABLE L1
Lookup table of IDs for Prognostic set genes

| NPI-ES | | |
|---|---|---|
| Probe_ID | GenBank | Unigene |
| 200853_at | NM_002106.1 | Hs.119192 |
| 201483_s_at | BC002802.1 | Hs.79058 |
| 201487_at | NM_001814.1 | Hs.10029 |
| 201890_at | NM_001034.1 | Hs.75319 |
| 202095_s_at | NM_001168.1 | Hs.1578 |
| 202188_at | NM_014669.1 | Hs.155314 |
| 202580_x_at | NM_021953.1 | Hs.239 |
| 202833_s_at | NM_000295.1 | Hs.297681 |
| 203362_s_at | NM_002358.2 | Hs.79078 |
| 203510_at | BG170541 | Hs.316752 |
| 203687_at | NM_002996.1 | Hs.80420 |
| 203764_at | NM_014750.1 | Hs.77695 |
| 204444_at | NM_004523.2 | Hs.8878 |
| 204603_at | NM_003686.1 | Hs.47504 |
| 204623_at | NM_003226.1 | Hs.82961 |
| 204766_s_at | NM_002452.1 | Hs.388 |
| 205240_at | NM_013296.1 | Hs.278338 |
| 206110_at | NM_003536.1 | Hs.70937 |
| 206461_x_at | NM_005951.1 | Hs.2667 |
| 208433_s_at | NM_017522.1 | Hs.54481 |
| 208546_x_at | NM_003524.1 | Hs.249216 |
| 208581_x_at | NM_005952.1 | Hs.374950 |
| 208767_s_at | AW149681 | Hs.296398 |
| 209040_s_at | U17496.1 | Hs.180062 |
| 209114_at | AF133425.1 | Hs.38972 |
| 209398_at | BC002649.1 | Hs.7644 |
| 209806_at | BC000893.1 | Hs.247817 |
| 209832_s_at | AF321125.1 | Hs.122908 |
| 209924_at | AB000221.1 | Hs.16530 |
| 210052_s_at | AF098158.1 | Hs.9329 |
| 210559_s_at | D88357.1 | Hs.334562 |
| 210792_x_at | AF033111.1 | Hs.112058 |
| 211456_x_at | AF333388.1 | Hs.367850 |
| 212094_at | BE858180 | Hs.137476 |
| 212141_at | X74794.1 | Hs.154443 |
| 212185_x_at | NM_005953.1 | Hs.118786 |
| 212484_at | BF974389 | Hs.18686 |
| 212613_at | AI991252 | Hs.87497 |
| 213245_at | AL120173 | Hs.301663 |
| 213892_s_at | AA927724 | Hs.28914 |
| 214472_at | NM_003530.1 | Hs.143042 |
| 214614_at | AI738662 | Hs.37035 |
| 214768_x_at | BG540628 | Hs.406565 |
| 215214_at | H53689 | Hs.405944 |
| 217165_x_at | M10943 | Hs.381097 |
| 217755_at | NM_016185.1 | Hs.109706 |
| 218350_s_at | NM_015895.1 | Hs.234896 |
| 218447_at | NM_020188.1 | Hs.6879 |
| 218542_at | NM_018131.1 | Hs.14559 |
| 218875_s_at | NM_012177.1 | Hs.272027 |
| 219061_s_at | NM_006014.1 | Hs.18212 |
| 219493_at | NM_024745.1 | Hs.123253 |
| 219555_s_at | NM_018455.1 | Hs.283532 |
| 219650_at | NM_017669.1 | Hs.89306 |
| 220085_at | NM_018063.1 | Hs.203963 |
| 220238_s_at | NM_018846.1 | Hs.26481 |
| 221436_s_at | NM_031299.1 | Hs.30114 |
| 221521_s_at | BC003186.1 | Hs.433180 |
| 221539_at | AB044548.1 | Hs.433317 |
| 222037_at | AI859865 | Hs.319215 |
| 201236_s_at | NM_006763.1 | Hs.75462 |
| 210576_at | AF133298.1 | Hs.268554 |

1. A method for determining the prognosis of a patient with breast cancer, the method comprising assigning a prognosis to the patient based on the expression levels in a breast tumour of said patient of a prognostic set of genes, wherein the prognostic set includes at least 5 genes from Table S6, said method optionally comprising the step of determining as least one status selected from the group consisting of estrogen receptor (ER) status and Erb2 status of the tumour sample.

2. A method according to claim 1, wherein the prognostic set comprises at least 10, 20, 30, 40, 50, 60 or all of the genes of Table S6.

3-4. (canceled)

5. A method according to claims 1 comprising the steps of:
(a) obtaining a breast tumour sample from the patient; and
(b) measuring the expression levels in the sample of the genes of the prognostic set.

6. A method according to claim 5 wherein step (b) comprises contacting said expression products obtained from the sample with a plurality of binding members capable of binding to expression products that are indicative of the expression of genes of the prognostic set, wherein such binding may be measured.

7. A method according to claim 6 wherein the binding members are complementary nucleic acid sequences or specific antibodies.

8. A method according to claims 1, comprising classifying the sample of breast tumour as being of either high NPI or low NPI, or as either of good or bad prognosis.

9. A method according to claim 1, wherein the step, of assigning a prognosis is carried out by comparing the expression profile from the breast tumour sample under test with previously obtained profiles and/or a previously determined standard profile which is characteristic of a particular prognosis.

10. A method according to claim 9 wherein the previously obtained profiles are stored as a database of profiles.

11. A method according to claim 1, further comprising comparing the expression levels of the prognostic set in the breast tumour sample before and after treatment to detect a change in the expression profile indicative of an improved prognosis or worsened prognosis.

12. Apparatus for assigning a prognosis to a breast tumour sample, which apparatus comprises a solid support to which are attached a plurality of binding members, each binding member being capable of specifically and independently binding to an expression product of one of a prognostic set of genes, wherein the prognostic set includes at least 5 genes from Table S6.

13. Apparatus according to claim 12, wherein the prognostic set comprises at least 5, 10, 20, 30, 40, 50, 60 or all of the genes of Table S6.

14. Apparatus according to claim 12 wherein the solid support has attached thereto only binding members that are capable of specifically and independently binding to expression products of the genes identified in Table S6.

15. Apparatus according to claim 12, comprising a nucleic acid microarray wherein the binding members are nucleic acid sequences.

16. A kit for assigning a prognosis to a patient with breast cancer, said kit comprising a plurality of binding members capable of specifically binding to expression products of genes of a prognostic set of genes and a detection reagent, wherein the prognostic set includes at least 5 genes from Table S6, said kit optionally comprising the apparatus of claim 12.

17. A kit according to claim 16, wherein the prognostic set comprises at least 10, 20, 30, 40, 50, 60 or all of the genes of Table S6.

18. A kit according to claim 16, further comprising a data analysis tool, wherein the data analysis tool is a computer program.

19. A kit according to claim 18 wherein the data analysis tool comprises an algorithm adapted to discriminate between the expression profiles of tumours with differing prognoses.

20. A kit according to claim 16 comprising expression profiles from breast tumour-samples with known prognoses and/or expression profiles characteristic of a particular prognosis.

21. (canceled)

22. A kit according to claim 16, comprising nucleotide primers capable of binding to the expression products of the genes of the prognostic set such that they can be amplified in a PCR.

23. A method of producing a nucleic acid expression profile for a breast tumour sample comprising the steps of
(a) isolating expression products from said breast tumour sample;
(b) identifying the expression levels of a prognostic set of genes, wherein the prognostic set of genes comprises at least 5 genes from Table S6; and
(c) producing from the expression levels an expression profile for said breast tumour sample.

24. A method according to claim 23, wherein the prognostic set comprises at least 10, 20, 30, 40, 50, 60 or all of the genes of Table S6.

25. A method according to claim 23 comprising adding the expression profile to a gene expression profile database.

26. A method according to claim 23 further comprising comparing the expression profile with a second expression profile or a plurality of second expression profiles characteristic of a particular prognosis.

27. A method according to claim 26, comprising the steps of:
(a) isolating expression products from a first breast tumour sample; contacting said expression products with a plurality of binding members capable of specifically and independently binding to expression products of the prognostic set; and creating a first expression profile from the expression levels of the prognostic set in the tumour sample;
(b) isolating expression products from a second breast tumour sample of known prognosis; contacting said expression products with a plurality of binding members capable of specifically and independently binding to expression products of the prognostic set of step (a), so as to create a comparable second expression profile of a breast tumour sample;
(c) comparing the first and second expression profiles to determine the prognosis of the first breast tumour sample.

28. An expression profile database comprising a plurality of gene expression profiles of breast tumour samples, wherein the gene expression profiles are derived from the expression levels of a prognostic set of genes, wherein the prognostic set of genes comprises at least 5 genes from Table S6, which database is retrievably held on a data carrier.

29. An expression profile database according to claim 28, wherein the prognostic set comprises at least 10, 20, 30, 40, 50, 60 or all of the genes of Table S6.

30. An expression profile database according to claim 28, wherein the expression profiles are nucleic acid expression profiles.

31. An expression profile database according to claim 28, wherein the expression profiles are categorised according to the ER status of the source tumour.

32. A method for identifying a set of genes that are differentially expressed within a group of tumours, the method including providing an expression profile from each of a plurality of tumours of the group, classifying the profiles according to molecular subtype of tumour, and analysing expression profiles within a subtype to identify a discriminating set of genes, wherein the genes of the discriminating set are differentially expressed within that subtype.

33. A method according to claim 32 further comprising steps to assign a class to a tumour sample from a patient, wherein differential expression of genes of the discriminating set are characteristic of the class, the steps including providing expression levels in the sample of the discriminating set, and assigning a class to the tumour based on the expression levels.

34. A method according to claim 32 comprising the steps of determining the expression levels of the genes of the discriminating set in a tumour sample, determining an expression profile from the expression levels and adding the profile to a database.

35. A method according to claim 32 wherein the molecular subtype of the tumour sample is also identified and added to the database.

36. A method according to claim 32 comprising providing expression profiles from the tumour at different stages of treatment and said expression profiles to determine a change in prognostic class, wherein the expression profiles are derived from the expression levels of genes of the discriminating set.

37. A method according to claim 32 wherein the tumours are breast tumours and the molecular subtype corresponds to the ER status of the tumour.
